(19) United States
(12) Patent Application Publication
(10) Pub. No.: US 2023/0404610 A1
(43) Pub. Date: Dec. 21, 2023

(54) SYSTEMS AND METHODS FOR MULTIDIRECTIONAL ARTICULATION

(71) Applicant: Interscope, Inc., Whitinsville, MA (US)

(72) Inventors: Jeffery B. Ryan, Whitinsville, MA (US); Corey Libby, Whitinsville, MA (US)

(73) Assignee: Interscope, Inc., Whitinsville, MA (US)

(21) Appl. No.: 18/251,519

(22) PCT Filed: Nov. 2, 2021
(86) PCT No.: PCT/US2021/057789

§ 371 Date: May 2, 2023

Related U.S. Application Data
(60) Provisional application No. 63/108,647, filed on Nov. 2, 2020.
(60) Provisional application No. 63/192,449, filed on May 24, 2021.
(60) Provisional application No. 63/192,468, filed on May 24, 2021.
(60) Provisional application No. 63/192,435, filed on May 24, 2021.

(51) Int. Cl. A61B 17/32

(52) U.S. Cl. CPC A61B 17/32002; A61B 2217/005; A61B 2017/00323

(57) ABSTRACT

The present disclosure includes systems and methods of surgical instruments with multidirectional articulation. The present disclosure includes systems and methods for an integrated steerable instrument. The present disclosure includes systems and methods for a steerable overtube instrument for maneuvering to a treatment site.

### CROSS-REFERENCE TO RELATED APPLICATIONS

The present application claims the benefit of priority to U.S. Provisional Application No. 63/108,647, filed Nov. 2, 2020; U.S. Provisional Application No. 63/192,435, filed May 24, 2021; U.S. Provisional Application No. 63/192,4499 filed May 24, 2021; and U.S. Provisional Application No. 63/192,468, filed May 24, 2021; the disclosures of which are incorporated herein by reference in their entireties.

### BACKGROUND

A steerable instrument may be inserted into an organ or a cavity of a body for examination. A doctor or surgeon may examine or observe the organ or the cavity and indicate a substance for removal, such as polyps, necrotic material, or any other material. The steerable instrument may cut a portion of the substance and cut the portion of the substance from the body.

### SUMMARY

At least one aspect relates to a surgical instrument. The surgical instrument can include an outer tubing extending from a proximal end to a distal end along an axis, the distal end comprising, articulation member of the outer tubing. The surgical instrument can include one or more articulation wires extending along the outer tubing and coupled to the articulation member. The surgical instrument can include a cutting assembly coupled to the distal end of the outer tubing, the cutting assembly including an outer component and an inner component disposed within the outer component coupled to the articulation member, the outer component defining a cutting window configured to cut material. The surgical instrument can include a flexible torque component having a portion disposed within the outer tubing, the flexible torque component coupled to the inner component and configured to rotate the inner component relative to the outer component to cut the material. The surgical instrument can include a handle comprising a first actuator to rotate the cutting assembly about the axis and a second actuator coupled to the one or more articulation wires to articulate the articulation member coupled to the cutting assembly away from the axis.

In some embodiments, wherein the axis is a first axis, and wherein the inner component rotates about a second axis relative to the first axis, the second axis formal by articulating the articulation member.

In some embodiments, the surgical instrument further comprises a tensioning rod disposed along the outer tubing, the tensioning rod configured to maintain a tension of the one or more articulation wires to control rotation and articulation of the articulation member coupled to the cutting assembly.

In some embodiments, the surgical instrument further comprises a flexible torque component having a portion disposed within the outer tubing, the flexible torque component coupled to the inner component and configured to mate the inner component relative to the outer component to cut the material.

In some embodiments, the surgical instrument further comprises an aspiration channel having an aspiration port configured to engage with a vacuum source, the aspiration channel partially defined by the flexible torque component and extending from the cutting window defined by the cutting assembly to the aspiration port.

In some embodiments, the first actuator is configured to articulate the distal end at first angle proportional to a second angle of rotation of the first actuator.

In some embodiments, the second actuator is a plurality of actuators, each of the plurality of actuators coupled to a corresponding wire of the one or more articulation wires.

In some embodiments, the surgical instrument further comprises a sheath enclosing the one or more articulation wires.

In some embodiments, the handle further comprises a locking assembly configured to restrict movement of at least one of the one or more articulation wires to set the cutting assembly to a predetermined articulation.

In some embodiments, the one or move articulation wires are a first set of one or more articulation wires. In some embodiments, the surgical instrument further comprises a second set of one or more articulation wires oriental at a first angle relative to the first set of one or more articulation wires. In some embodiments, a third set of one or more articulation wires oriented at a second angle relative to the first set of one or more articulation wires. In some embodiments, the second actuator is coupled to the first, second, and third set of one or more articulation wires.

At least one aspect relates to a surgical instrument. The surgical instrument can include an outer tubing extending from a proximal end to a distal end along an axis, the distal end comprises a plurality of segments. The surgical instrument can include one or more articulation wires extending along the outer tubing and coupled to the plurality of segments. The surgical instrument can include a cutting assembly coupled the distal end of the outer tubing, the cutting assembly configured to cut material from a subject. The surgical instrument can include a handle comprising a first actuator to rotate a first component of the cutting assembly about the axis and a second actuator coupled to the one or more articulation wires to selectively articulate at least one of the plurality of segments coupled to the cutting assembly away from the axis.

In some embodiments, wherein the first component is an outer component of the cutting assembly and the cutting assembly further comprises an inner component disposed within the outer component, the outer component defining a cutting window.

In some embodiments, the surgical instrument further comprises a tensioning rod disposed along the outer tubing, the tensioning rod configured to maintain a tension of the one or more articulation wises to control rotation and articulation of the articulation member coupled to the cutting assembly.

In some embodiments, the surgical instrument further comprises a flexible torque component having a portion disposed within the outer tubing, the flexible torque component coupled to the inner component and configured to rotate the inner component relative to the outer component to cut the material.

In some embodiments, the surgical instrument further comprises an aspiration channel having an aspiration port configured to engage with a vacuum source, the aspiration channel partially defined by the flexible torque component and extending fount the cutting window defined by the cutting assembly to the aspiration port.

In some embodiments, wherein the flat actuator is configured to articulate the distal end at first angle proportional to a second angle of rotation of the first actuator.

In some embodiments, the second actuator is a plurality of actuators, each of the plurality of actuators coupled to a corresponding wire of the one or more articulation wires.

In some embodiments, the surgical instrument further comprises a sheath enclosing the one or more articulation wires.

In some embodiments, wherein the handle further comprises a locking assembly configured to restrict movement of the one or more articulation wires to set the cutting assembly to a predetermined articulation.

In some embodiments, wherein the outer tubing includes a plurality of securing elements extending from the plurality of segments, the plurality of securing elements configured to secure the one or more articulation wires to the outer tubing.

In some embodiments, the surgical instrument further comprises a sheath enclosing the one or more articulation wires and the plurality of securing elements.

In some embodiments, wherein the one or more articulation wires are a first set of one or more articulation wires. The surgical instrument can include a second set of one or more articulation wires oriented at a first angle relative to the first set of one or more articulation wires. The surgical instrument can include a third set of one or more articulation wires oriented at a second angle relative to the first set of one or more articulation wires. The second actuator can be coupled to the first, second, and third set of one or more articulation wires.

At least one aspect relates to a method of retrieving material from a subject. The method can include inserting a surgical instant into the subject to cut the material from the subject, the surgical instrument including an outer tubing extending from a proximal end to a distal end along an axis, the distal end coupled to a cutting assembly, the cutting assembly coupled to one or more articulation wires extending along the outer tubing. The method can include applying a first control input to a first actuator coupled to a handle to rotate the cutting assembly about the axis. The method can include applying a second control input to a second actuator coupled to the one or more articulation wires to articulate the cutting assembly away from the axis.

In some embodiments, applying the first control input comprises rotating the rust actuator to rotate the cutting assembly about the axis.

In some embodiments, applying the first control input comprises rotating the first actuator at a first angle about the axis to rotate the cutting assembly at the first angle about the axis.

In some embodiments, inserting the surgical instrument comprises inserting the surgical instrument into the subject to cut the mated from the subject, the surgical instrument including the outer tubing extending front the proximal end to the distal end along the axis, the distal end coupled to the cutting assembly, the cutting assembly coupled to a first set et one or more articulation wires extending along the outer tubing and a second set of one or more articulation wires oriented at a first angle relative to the first set of one or more articulation wires.

In some embodiments, applying the second control input includes applying the second control input to the second actuator coupled to the first set of one or more articulation wires to articulate the cutting assembly away from the axis in a first direction. In some embodiment, applying the second control input includes applying a third control input to third actuator coupled to a second set of one or more articulation wires to articulate the cutting assembly away from the axis in a second direction, the second direction opposite to the first direction.

In some embodiments, the method includes modifying a tension of the one or more articulation wires to modify rotation and articulation of the cutting assembly.

At least one aspect relates to a surgical instrument. The surgical instrument can include a first telescopic tubing having a first diameter, the first telescopic tubing partially enclosing a second telescopic tubing, the second telescopic tubing extending out atilt first telescopic tubing, the second telescopic tubing having a wood diameter less than the rust diameter, the second telescopic tubing configured to retract into the first telescopic tubing. The surgical instrument can include a third telescopic tubing partially enclosed by the second telescopic tubing and extending out of the second telescopic tubing, the third telescopic tubing hawing a third diameter less than the second diameter, the third telescopic tubing configured to retract into the second telescopic tubing. The surgical instrument can include an actuator coupled to the third telescopic tubing, the actuator configured to expand the third telescopic tubing out of second telescopic tubing. The surgical instrument can include a cutting assembly coupled to the third telescopic tubing, the cutting assembly configured to cut material from a subject.

In some embodiments, the first telescopic tubing extends along an axis, wherein the second telescopic tubing extends along a first curve relative to the axis, and wherein the third telescopic tubing extends along a second curve relative to the axis.

In some embodiments, the cutting assembly comprises an outer component and an inner component disposed within the outer component, the outer component defining a cutting window.

In some embodiments, the surgical instrument further comprises a flexible torque component having a portion disposed within the first, second, and third telescopic tubing, the flexible torque component coupled to the inner component and configured to rotate the inner component relative ea the outer component to cut the material.

In some embodiments, the surgical instrument further comprises an aspiration channel having an aspiration port configured to engage with a vacuum source, the aspiration channel partially defined by the flexible torque component and the first, second, and third telescopic tubing, the aspiration channel extending from the cutting window defined by the cutting assembly to the aspiration port.

At least one aspect is directed to a method of retrieving material from a subject. The method can include inserting a surgical instrument into the subject to cut material from the subject, the surgical instrument including a first telescopic tubing having a proximal end and a distal end, the first telescopic tubing having a first diameter, the distal end of the first telescopic tubing coupled to a cutting assembly. The method can include applying a first control input to a first actuator to extend the distal end or the first telescopic tubing out of a distal end of a second telescopic tubing, the second telescopic tubing including a distal end enclosing the proximal end of the first telescopic tubing, the second telescopic tubing having a second diameter greater than the first diameter. The method can include applying a second control input to the first actuator to extend the distal end of the second telescopic tubing out of a distal end of a third telescopic tubing, the third telescopic tubing including a distal end enclosing a proximal end of the second telescopic tubing, the third telescopic tubing having a third diameter greater than the second diameter, the third telescopic tubing including a proximal end coupled to the first actuator. The method can include applying third control input to a second actuator to actuate the cutting assembly to retrieve the material.

At least one aspect relates to a steerable instrument. The steerable instrument can include a cutting assembly configured to cut material from a subject, the cutting assembly comprises an outer sheath and an inner sheath disposed within the onto sheath, the outer sheath defining a cutting window. The steerable instrument can include a flexible outer tubing having an outer diameter less than 4 mm, the flexible outer tubing extending from a proximal end of the flexible outer tubing to a distal end of the flexible outer tubing, the distal end of the flexible outer tubing coupled to the outer sheath, the flexible outer tubing configured to receive torque at the proximal end of the flexible outer tubing and transmit the torque to the outer sheath to rotate the outer sheath. The steerable instrument can include a first connector coupled to the proximal end of the flexible outer tubing, the first connector configured to articulate the distal end of the flexible outer tubing relative to a longitudinal axis extending through the steerable instrument responsive to receiving a first control input at the first connector. The steerable instrument can include a flexible torque component disposed within the flexible outer tubing, the flexible torque component coupled to the inner sheath, the flexible torque component configured to rotate a the inner sheath relative to the outer sheath to cut the material. The steerable instrument can include a second connector coupled to the proximal end of the flexible outer tubing and configured to rotate the flexible torque component responsive to receiving a second control input at the second connector to cause the inner sheath to rotate relative to the outer sheath to cut the material. The steerable instrument can include an aspiration channel having an aspiration port configured to engage with a vacuum source, the aspiration channel partially defined by the flexible torque component and extending from the cutting window defined by the cutting assembly to the aspiration port.

In some embodiments, the first connector is configured to articulate the distal end of the flexible outer tubing relative to a first angle to the longitudinal axis, the first angle proportional to a second angle of the first control input at the first connector.

At least one aspect relates to a method of retrieving material from a subject. The method can include inserting a surgical tool into the subject. The method can include disposing a steerable instrument within a working channel of the surgical tool to cut material from the subject, the steerable instrument including a cutting assembly configured to cut the material, the cutting assembly comprises an outer sheath and an inner sheath disposed within the outer sheath, the outer sheath defining a cutting window, the cutting assembly coupled to a flexible outer tubing, the flexible outer tubing having an outer diameter less than 4 mm. The method can include applying a first control input to a first connector coupled to the proximal end of the flexible outer tubing to cause articulation of the distal end of the flexible outer tubing relative to a longitudinal axis extending through the steerable instrument. The method can include applying a second control input to a second connector coupled to the proximal end of the flexible outer tubing to rotate a flexible torque component disposed within the flexible outer tubing, the flexible torque component coupled to the inner sheath, the flexible torque component configured to rotate the inner sheath relative to the outer sheath to cut the material. The method can include actuating a vacuum source coupled to the steerable instrument to provide suction through an aspiration channel defined by an inner wall of the steerable instrument to cut the material from the subject via the aspiration channel.

As least one aspect relates to a steerable instrument. The steerable instrument can include a cutting assembly configured to cut material from a subject, the cutting assembly comprises an outer sheath and an inner sheath disposed within the outer sheath, the outer sheath defining a cutting window. The steerable instrument can include a flexible outer tubing having an outer diameter less than 6 mm, the flexible outer tubing extending from a proximal end of the flexible outer tubing to a distal end of the flexible outer tubing, the distal end of the flexible outer tubing coupled to the outer sheath, the flexible outer tubing configured to receive a torque at the proximal end of the flexible outer tubing and transmit the torque to the outer sheath to rotate the outer sheath. The steerable instrument can include a first connector coupled to the proximal end of the flexible outer tubing, the first connector configured to articulate the distal end or the flexible outer tubing relative to a longitudinal axis extending through the steerable instrument responsive to receiving a first control input at the first connector. The steerable instrument can include a flexible torque component having a portion disposed within the flexible outer tubing, the flexible torque component coupled to the cutting assembly and configured to rotate the inner sheath relative to the outer sheath to cut the material. The steerable instrument can include a second connector coupled to the proximal end of the flexible outer tubing and configured to rotate the flexible torque component responsive to receiving a second control input at the second connector to cause the inner sheath to rotate relative to the outer sheath to cut the material. The steerable instrument can include an aspiration channel having an aspiration port configured to engage with a vacuum source, the aspiration channel partially defined by the flexible torque component and extending from the cutting window defined by the cutting assembly to the aspiration port. The steerable instrument can include at least one attachment member configured to attach the steerable instrument to a surgical tool.

In some embodiments, the first connector is configured to articulate the distal end of the flexible outer tubing relative to a first angle to the longitudinal axis, the first angle proportional to a second angle of the first control input at the first connector.

In some embodiments, the at least one attachment member is a first attachment member disposed at a distal end of the surgical tool, and further comprises a second attachment member disposed at a proximal end of the surgical tool.

In some embodiments, the at least one attachment member includes a locking mechanism to pure the at least one attachment member to the surgical tool.

In some embodiments, the at least one attachment member is an elastic band.

In some embodiments, the at least one attachment member includes an opening configured to teethe the steerable instrument.

At least one aspect relates to a shod of performing a surgery. The method can include positioning a plurality of attachment members along a surgical tool, each attachment member configured to receive a steerable instrument. The method can include maneuvering the steerable instrument through each of the plurality of attachment members along surgical tool to attach the steerable instilment to the surgical tool, the steerable instrument including a cutting assembly configured to cut material from a subject, the cutting assembly comprises an outer sheath and an inner sheath disposed within the outer sheath, the tooter sheath defining a cutting window, the cutting assembling coupled to a flexible outer tubing. The method can include insetting the surgical tool and the steerable instrument into the subject. The method can include positioning, by a control input applied to a first connector coupled to the proximal end of the flexible outer tubing, the distal end of the flexible outer tubing to a position in the subject in which the opening of the cutting window is at the material and viewable via a camera of the surgical tool. The method can include applying a second control input to a second connector coupled to the proximal end of the flexible outer tubing to rotate a flexible torque component disposed within the flexible outer tubing, the flexible torque component coupled to the inner sheath, the flexible torque component configured to rotate the inner sheath relative to the outer sheath to cut the material. The method can include actuating a vacuum source coupled to the surgical tool to prof de suction to an aspiration channel defined by an inner wall of the steerable instrument to cut the material from the subject via the aspiration channel. The method can include removing, from the subject, the steerable instrument through each of the plurality of attachment members along the surgical tool.

At least one aspect relates to a steerable instrument. The steerable instrument can include a steerable tubing within which surgical instrument is disposed, the steerable tubing extending from a proximal end of the wearable tubing to a distal end of the steerable tubing. The surgical instrument can include a cutting assembly configured to cut material from a subject, the cutting assembly comprises an outer sheath and an inner sheath disposed within the outer sheath, the outer sheath defining a cutting window. The surgical instrument can include a flexible tubing extending from the proximal end of the steerable tubing to the distal end of the steerable tubing, the distal end of the flexible tubing coupled to the outer sheath. The surgical instrument can include a first connector coupled to the proximal end of the steerable tubing, the first connector configured to articulate the distal end of the steerable tubing along a longitudinal axis extending through the steerable instrument responsive to receiving a first control input at the first connector. The surgical instrument can include a flexible torque component having a portion disposed within the flexible tubing, the flexible torque component coupled to the inner sheath and configured to rotate the inner sheath relative to the outer sheath to cut the material. The surgical instrument can include a second eon for coupled to the proximal end of the steerable tubing and configured to rotate the flexible torque component to rotate the inner sheath relative to the outer sheath responsive to receiving a second control input at the second connector. The surgical immanent can include an aspiration channel having an aspiration port configured to engage with a vacuum source, the aspiration channel partially defined by the flexible torque component and extending from the cutting window defined by the cutting assembly to the aspiration port.

In some embodiments, the first connector is further configured to rotate the flexible tubing.

In some embodiments, the first connector is configured to articulate the distal end of the flexible tubing along a first angle to the longitudinal axis, the first angle proportional to a second angle of the first control input at the first connector.

In some embodiments, the steerable instrument finer comprises a coating disposed between the flexible tubing and the steerable tubing.

In some embodiments, a diameter of the steerable tubing is less than 4.0 mm.

In some embodiments, a diameter of the flexible tubing is less than 3.1 mm.

At least one aspect relates to a method of retrieving material from a subject. The method can include inserting a steerable instrument into the subject to cut material from the subject, the steerable instrument including a steerable tubing within which a surgical immanent is disposed, the steerable tubing extending from a proximal end of the steerable tubing to a distal end of the steerable tubing, the surgical instrument including a cutting assembly configured to cut the material from the subject, the cutting assembly comprises an outer sheath and an inner sheath disposed within the outer sheath, the outer sheath defining a cutting window, the cutting assembly coupled to a distal end of a flexible tubing extending from the distal end of the steerable tubing to the proximal end of the steerable tubing. The method can include applying a first control input to a first connector coupled to the proximal end of the steerable tubing to cause articulation of the distal end of the steerable tubing along a longitudinal axis extending through the surgical instrument. The method can include applying a second control input to a second connector coupled to the proximal end of the steerable tubing to rotate a flexible torque component disposed within the flexible tubing, the flexible torque component coupled to the inner sheath, the flexible torque component configured to rotate the inner sheath relative to the outer sheath to cut the material. The method can include actuating a vacuum source coupled to the surgical instrument to provide suction through an aspiration channel defined by an inner wall of the steerable instrument to cut the material from the subject via the aspiration channel.

At least one aspect relates to a steerable instrument. The steerable instrument can include a steerable tubing that includes at least one attachment member configured to attach the steerable tubing to a surgical tool, the steerable tubing extending from a proximal end of the steerable tubing to a distal end of the steerable tubing, the steerable tubing comprises a surgical instrument. The surgical instrument can include a cutting assembly configured to cut material from a subject, the coning assembly comprises an outer sheath and an inner sheath disposed within the outer sheath, the outer sheath defining a cutting wince. The surgical instrument can include a flexible tubing extending from the proximal end of the steerable tubing to the distal end of the steerable tubing, a distal end of the flexible tubing coupled to the outer sheath. The surgical instrument can include a first connector coupled to a proximal end of the steerable tubing, the first connector configured to articulate the distal end of the steerable tubing along a longitudinal axis extending through the steerable tubing responsive to receiving a first control input at the first connector. The surgical instrument can include a flexible torque component having a portion disposed within the flexible tubing, the flexible torque component coupled to the inner sheath and von figured to rotate the inner sheath relative to the outer sheath to cut the material. The surgical instrument can include a second connector coupled to the proximal end of the gable tubing and configured to rotate the flexible torque component to rotate the inner sheath relative to the outer sheath responsive to receiving a second control input at the second connector. The surgical instrument can include an aspiration channel having an aspiration port configured to engage with a vacuum source, the aspiration channel partially defined by the flexible torque component and extending front the cutting window defined by the cutting assembly to the aspiration port.

In some embodiments, the at least one attachment member is a first attachment member disposed at a distal end of the surgical tool, and further comprises a second attachment member disposed at a proximal end of the surgical tool.

In some embodiments, wherein the at least one attachment member include a locking mechanism to secure the at least one attachment member to the surgical tool.

In some embodiments, wherein the at least one attachment member is an elastic band.

In some embodiments, wherein the at least one attachment member includes an opening configured to receive the steerable instrument.

In some embodiments wherein the first connector is further configured to rotate the flexible tubing.

In some embodiment wherein the first connector is configured to articulate the distal end of the flexible tubing along a first angle to the longitudinal axis, the first angle proportional to a second angle of the first control input at the first connector.

In some embodiments, the steerable instrument includes a coating disposed between the flexible tubing and the steerable tubing.

In some embodiments, a diameter of the steerable tubing is less than 4.0 mm.

In some embodiments, a diameter of the flexible tubing is less than 3.1 mm.

At least one aspect relates to a method of performing a surgery. The method can include positioning a plurality of attachment members along a surgical tool, each attachment member configured to receive a steerable tubing within which a steerable instrument is disposed. The method can include maneuvering the steerable tubing through each artist plurality of attachment members along the surgical tool to attach the steerable tubing to the surgical tool, the steer able instrument can include a cutting assembly configured to cut material from a subject, the cutting assembly comprises an outer sheath end an inner sheath disposed within the outer sheath, the outer sheath defining a cutting window, the cutting assembly coupled to a flexible tubing extending from a proximal end of the stable tubing to a distal end of the steerable tubing. The method can include inserting the surgical tool and the steerable tubing into the subject to cut the material from the subject. The method can include positioning, by a control input applied to a first connector coupled to the proximal end of the steerable tubing, the distal end of the steerable tubing to a position in the subject in which the opening of the cutting window is at the material and viewable via a camera of t surgical tool. The method can include applying a second control input to a second connector coupled to the proximal end of the steerable tubing to rotate a flexible torque component disposed within the flexible tubing, the flexible torque component coupled to the inner sheath, the flexible torque component configured to rotate the inner sheath relative to the outer sheath to cut the material. The method can include actuating a vacuum source coupled to the surgical tool to provide suction to an aspiration channel defined by an inner wall of the steerable instrument to cut the material from the subject via the aspiration channel. The method can include removing, from the subject, the steerable instrument through each of the plurality of attachment members along the surgical tool.

These and other aspects and implementations are discussed in detail below. The tangoing information and the following detailed description include illustrative examples of various aspects and implementations, and provide an overview or framework for understanding the nature and character of the claimed aspects and implementations. The drawings provide illustration and a furor understanding of the various aspects and implementations, and are incorporated in and constitute a part of this specification.

### BRIEF DESCRIPTION OF THE FIGURES

The accompanying drawings are not intended to be drawn to scale. Like reference numbers and designations in the various drawings indicate like elements. For purposes of clarity, not every component can be labeled in every drawing. In the drawings:

FIGS. 1A-1D are diagram of procedures that can be performed according to embodiments of the present disclosure.

FIG. 2A-2D shows views of the surgical instrument for maneuvering to a treatment site according to embodiments of the present disclosure.

FIG. 3A-3D shows views of the surgical instrument with segments and securing elements for maneuvering to a treatment site during a laparoscopic or hysteroscopic procedure according to embodiments present disclosure.

FIG. 4 shows a perspective view of the surgical instrument with segments for maneuvering to a treatment site during a laparoscopic or hysteroscopic procedure according to embodiments of the present disclosure.

FIGS. 5A-5F show perspective views of a surgical instrument for maneuvering to a treatment site during a laparoscopic or hysteroscopic procedure according to embodiments of the present disclosure.

FIGS. 6A-6D show perspective views of a surgical instrument for maneuvering to a treatment site during a laparoscopic or hysteroscopic procedure according to embodiments of the present disclosure.

FIGS. 7A-7E show perspective views of a surgical instrument for maneuvering to a treatment site during a laparoscopic or hysteroscopic procedure according to embodiments of the present disclosure.

FIGS. 8A-8D show perspective views of a surgical instrument for maneuvering to a treatment site during a laparoscopic or hysteroscopic procedure according to embodiments of the present disclosure.

FIG. 9 is a diagram of a method of performing a laparoscopic or hysteroscopic procedure using the surgical instrument.

FIGS. 10A-10D show the surgical instrument with telescopic tubing for maneuvering to a treatment site during a laparoscopic or hysteroscopic procedure according to embodiments of the present disclosure.

FIGS. 11A and 11B show perspective views of a surgical instrument having a telescopic configuration for maneuvering to a treatment site during a laparoscopic or hysteroscopic procedure according to embodiments of the present disclosure.

FIG. 12 is a diagram of a method of performing a laparoscopic or hysteroscopic procedure using the steerable instrument.

FIGS. 13A-13D show views of the steerable instrument for maneuvering to a treatment site during a laparoscopic or hysteroscopic procedure according to embodiments of the present discloses.

FIG. 14 shows a view of a surgical tool for maneuvering the steerable instrument to a treatment site during a laparoscopic or hysteroscopic procedure according to embodiments of the present disclosure.

FIG. 15 shows a view of a surgical tool for maneuvering the steerable instrument to a treatment site during a laparoscopic or hysteroscopic procedure according to embodiments of the present disclosure.

FIG. 16 shows a view of a surgical instrument for maneuvering the steerable instrument to a treatment site during a laparoscopic or hysteroscopic procedure according to embodiments of the present disclosure.

FIG. 17 is a diagram of a method of performing a laparoscopic or hysteroscopic procedure using the steerable instrument.

FIG. 18 is a diagram of a method of performing a laparoscopic or hysteroscopic procedure using the steerable instrument with the attachment members.

FIGS. 19A-19D show views of the steerable instrument for manuvering to a treatment site during a laparoscopic or hysteroscopic procedure according to embodiments of the present disclosure.

FIG. 20 shows a view of a surgical tool for maneuvering the steerable instrument to a treatment site during a laparoscopic or hysteroscopic procedure according to embodiments of the present disclosure.

FIG. 21 shows a view of a surgical tool fin maneuvering the steerable instrument to a treatment site during a laparoscopic or hysteroscopic procedure according to embodiments of the present disclosure.

FIG. 22 shows a view of a surgical tool for maneuvering the steerable instrument to a treatment site dining a laparoscopic or hysteroscopic procedure according to embodiments of the present disclosure.

FIG. 23 is a diagram of a method of performing a laparoscopic or hysteroscopic procedure using the steerable instrument.

FIG. 24 is a diagram of a method of performing a laparoscopic or hysteroscopic procedure using the steerable instrument with the attachment members.

### DETAILED DESCRIPTION

The present disclosure will be more completely understood through the following description, which should be read in conjunction with the drawings. In this description, like mothers refer to similar elements within various embodiments of the present disclosure. Within this description, the claims will be explained with respect to embodiments. The skilled artisan will readily appreciate that the methods, apparatus, and systems described herein are merely exemplary and that variations can be made without departing front the spirit and scope of the disclosure.

For purposes of reading the description of the various embodiments below, the following descriptions of the sections of the specification and their respective contents may be helpful:
Section A describes an overview of material removal solutions which may be useful for practicing embodiments described herein.
Section B describes systems and methods of surgical instruments with multidirectional articulation.
Section C describes systems and methods of telescopic surgical instruments.
Section D describes system and methods for an integrated steerable instrument for maneuvering so a treatment site according to embodiments of the present disclosure.
Section E describes systems and methods for a steerable overtube instrument for maneuvering to a treatment site according to embodiments of the present disclosure.

### A. Material Removal Solutions Overview

Technologies provided herein are directed towards instruments that can efficiently and precisely cut materials, such as polyps, necrotic material, or other material, from a patient. In particular, the instruments is capable of maneuvering along a tortuous path to provide torque and rotation from a proximal end to a distal end of the instruments including the instruments and the cutting assembly. The instruments can be inserted into a cavity of the patient to cut, dissect, or penetrate materials. The instruments can retrieve cut samples such as polyps, necrotic material, or other material, without having to cut the instruments from the treatment site within the patient's body and resort to, for example, a suction device, a drilling deuce, or other laparoscopic or hysteroscopic procedures.

The material can be referred to as, and used interchangeably with, other descriptive terms, such as object, substance, or content within the subject. The material can be manifested overtime within the subject to clog or block the path or the opening of the caul. The material can be a liquid, a solid, or a combination of a liquid and a solid substance determined to be cut, extracted, examined, or collected from the subject. The subject can form the material in various procedures. For example, the material can be formed from a damage or an injury to the subject, such as a cut or a bruise. The platelets of the subject can receive an indication of the damaged subject. The platelets can fill in or plug the damaged portion of the subject. The platelets can release chemicals to attract additional platelets and other cells within the subject to block the damaged portion. One or more clotting factors (e.g., proteins) may tangle with the platelets to generate a net to trap more platelets and other cells, which can cause a clog or a blockage to the subject. The clogging of the blockage of the subject can refer to the material.

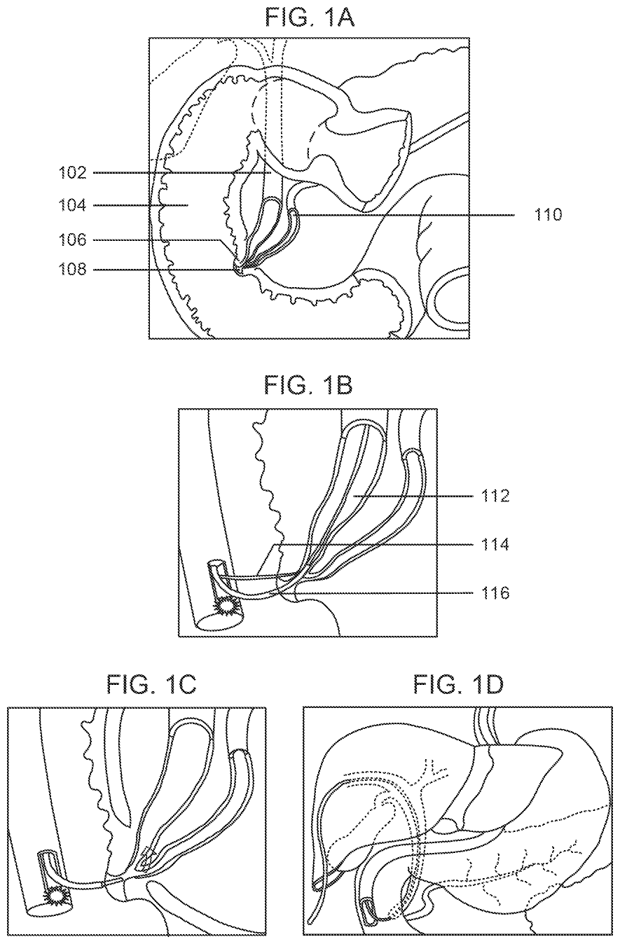

The instruments described herein can be used in various applications using the exemplary procedures described previously. Referring to FIGS. 1A-1D, shown are diagrams of procedures that can be performed using the instruments, FIG. 1A shows a bile duct 102, a descending duodenum 104, a major papilla of Vater 106, a common channel 108, and a pancreatic duct 110, FIG. 1B shows a sphincterotomy including a guide wire 112, a cutting wire 114, and a papillotome 116, FIG. 1C shows a precut sphincterotomy, and FIG. 1D shows a combined percutaneous-endoscopic procedure. The instruments can be utilized in a percutaneous procedure such as any medical procedure or method where access to inner organs or other material is done via needle-puncture of the shin. The instruments can include a 300-600 mm flexible catheter, a 3.0 mm lumen, and provide visualization via IR. The instruments can include a 30 mm steerable tip. An endorotor can maneuver with the cutting assembly within the treatment site. The instruments can be utilized in an endoscopic procedure to examine the interior of a hollow organ or cavity of the body. The instruments can be used in with an endoscopic retrograde cholangiopancreatography (ERCP) technique combining the use of endoscopy and fluoroscopy to diagnose and treat certain problems oleic biliary or pancreatic ductal systems. The instruments can assist the catheter in reaching the treatment site. The instruments can include a 3.0 mm lumen or visualization via IR.

The instruments can be a flexible hysteroscope. The instruments can be used as or with various types of flexible endoscopes, including, but not limited to, hysteroscopes, laparoscopes, bronchoscopes, gastroscopes, and laryngoscopes, or other medical devices that may be used to treat patients. The instruments procedures may be performed on various parts or portions of the body, such as the uterus, fallopian tubes, ovaries, ear, esophagus, vessels, stomach, small intestine, large intestine, pancreas, or other hollow portion of the subject. Various procedures can be performed using the material removal tool, such as a laparoscopy to inspect the outside of the uterus, ovaries, and fallopian tubes, as, for example, in the diagnosis of female infertility. Additionally, the material removal can acquire images of the treatment site from which material is to be cut as well as of the cutting assembly, as the cutting assembly is moved to the treatment site, allowing the instruments to be accurately delivered to the treatment site.

However, it is difficult to maneuver the cutting assembly to the desired material at the treatment site. For example, it can be difficult to cut relatively large portions of material adjacent to where the material protrudes from underlying tissue. In another example, tortuous pathways in the colon, pancreas, or duodenum may require several high-angle turns to reach the target site. As such, it can be technically challenging to navigate a distal end of the surgical instrument through the tortuous pathways (e.g., multiple bends in various directions), while retaining the ability of a cutting element or other tool at the distal end of the surgical instrument to be properly operated. For example, maneuvering the surgical instrument together with the cutting assembly can cause the cutting assembly to maneuver away horn the treatment site. Moreover, attempting to maneuver of the cutting assembly with the magical instrument can squeeze or damage the flexible torque coil, which limits the ability to actuate (e.g., rotate) the cutting assembly. The instruments described herein address these problems by enabling precise and accurate control of the distal end of the instruments to maneuver the cutting assembly to specific locations (e.g., material) independently of the rest of the instrument at the treatment site and/or the cavity of the subject.

B. Systems and Methods of Surgical Instruments with Multidirectional Articulation

A surgical instrument and methods thereof in accordance with the present disclosure can include components such as an outer tubing, a cutting assembly, articulation wires, a handle, a rotation actuator, a flexible torque component, an articulation actuator, and an aspiration channel. Generally, the surgical instrument may be used to provide treatment in narrow portions of a body, such as a uterus, fallopian tubes, ovaries, or in some cases, to provide non-surgical treatment to a subject. The surgical instrument may be guided to a treatment site to perform a laparoscopic or hysteroscopic procedure. For example, the operator may insert the surgical instrument into a cavity of the subject and articulate the cutting assembly to the material.

After the surgical instrument is at the treatment site, the operator can steer the cutting assembly to the material. The location of the material can refer to a treatment site, portion, or area for extraction, inspection, or performing other procedures using the surgical instrument. The cutting assembly can be configured to cut the material, and include an outer component and au inner component disposed within the outer component. The surgical instrument can include an articulation actuator configured to actuate the articulation wires to articulate the tubing along a longitudinal axis extending through the surgical instrument. The stagiest instrument can include a flexible torque component configured to rotate the inner component relative to the outer component to cut the material. The surgical instrument can include a rotation actuator configured to articulate the flexible torque component to cause the cutting assembly to cut the material. The surgical instrument can include an aspiration channel connected to a vacuum source configured to suction the material cut by the cutting assembly.

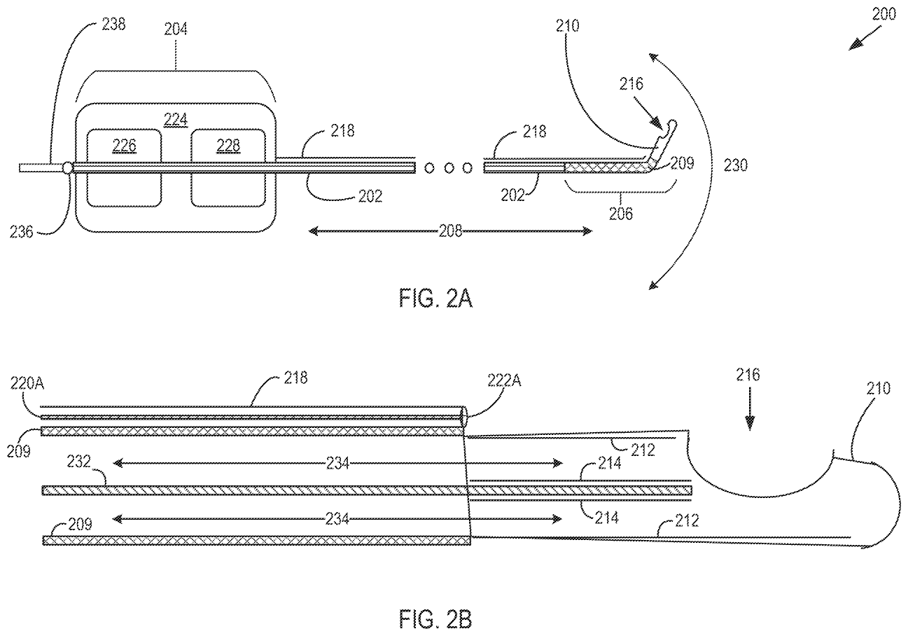

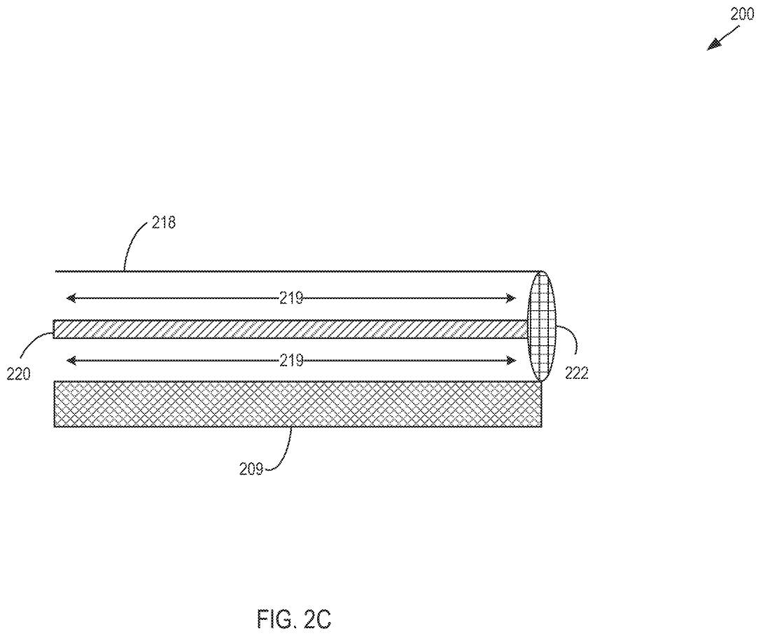

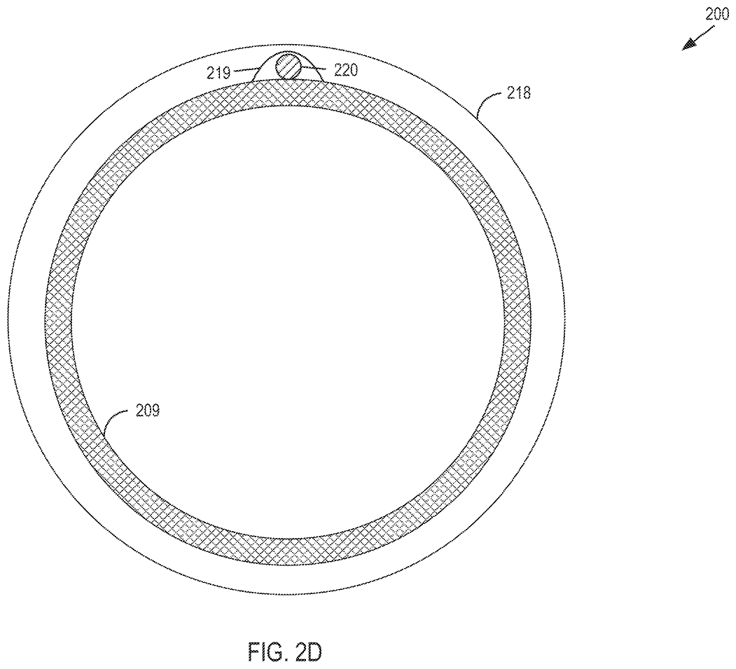

Referring to FIGS. 2A-2D, shown are views of the surgical instrument 200 for maneuvering to a treatment sift during a laparoscopic or hysteroscopic procedure according to embodiments of the present disclosure. The surgical instrument 200 can include an outer tubing 202. The outer tubing 202 can include a proximal end 204, a longitudinal axis 208, and an articulation member 209. The surgical instrument 200 can include a cutting assembly 210, which tan include an outer component 212 and an inner component 214. The outer component 212 can define a cutting window 216. The surgical instrument 200 can include a tubing sheath 218, wire channel 219, articulation wires 220, wire couplers 222, a handle 224, rotation actuator 226, an articulations actuator 228, an articulation axis 230, a flexible torque component 232, an aspiration channel 234, and an aspiration port 236 configured to couple to an external tubing 238.

Including the articulation member 209 can be advantageous to allow for plow deployment of the surgical instrument 200 to a treatment site along with articulation of the surgical instrument 200, such as by enabling the distal end 206 to have a different flexibility or malleability relative to the rest of the outer tubing 202. In particular, the articulation member 209 can have more flexibility or malleability relative to the outer tubing 202. For example, pulling of the articulation wire 220 coupled to the wire couplers 222 can cause more articulation of the articulation member 209 while enabling the outer tubing 202 to maintain its stiffness or structure in the body, which can allow the operator of the surgical instrument 200 to more easily articulate the cutting window 216 to the treatment site. Conversely, the articulation member 209 can have less flexibility or malleability relative to the outer tubing 202. For example, pulling of the articulation wire 220 coupled to the wire couplers 222 can cause less articulation of the articulation member 209 while enabling the outer tubing 202 to bend in the body to reach the treatment site, which can enable the operator of the surgical instrument 200 to reach the treatment site and carefully articulate the cutting window 216 to locations within the treatment site.

For example, referring further to FIG. 2A, for performing a procedure to cut material from the treatment site, the outer tubing 202 can be introduced into a cavity of the submit. The cutting assembly 210 can be introduced to the treatment site. The operator can use the rotation actuator 226 to rotate the cutting assembly 210 about the longitudinal axis 208 to the material. The operator can use the articulation actuator 228 to articulate the cutting assembly 210 around the articulation axis 230. A motor, rotation actuator 226, or the articulation actuator 228 can actuate the cutting assembly 210 to cut the material. The material may be extracted, cut, collected, or investigated by the surgical instrument 200. In some cases, the cutting assembly 210 can extract, pull, or collect the material into the cutting window 216. The Vacuum source can suction the material into the aspiration channel 234 extending from the cutting window 216 to the aspiration port 236.

Referring to FIG. 2A in conjunction with FIG. 2B, the outer tubing 202 can include the proximal end 204, the distal end 206, and the longitudinal axis 208. The outer tubing 202 can extend from the proximal end 204 to the distal end 206. The proximal end 204 can refer to the base, the beginning, or the foundation of the outer tubing 202. The distal end 206 can refer to the tip or the front of the outer tubing 202. The longitudinal axis 208 can extend through the surgical instrument 200.

The articulation member 209 can be located at the distal end 206 of the outer tubing 202. The articulation member 209 can be configured to have additional malleability or flexibility relative to the outer tubing 202. For example, the articulation member 209 can be a woven section of the outer tubing 202. In another example, the articulation member 209 can be a braid or braided sheath. The additional malleability or flexibility of the articulation member 209 enables the articulation of the distal end 206 within the subject and along the longitudinal axis 208.

The outer tubing 202 can be a navigation wire, a motorized wire, or a braid. The outer tubing 202 can include nitinol, flexible plastic, rubber, cloth, metal, steel, titanium, nickel, or carbon fiber. The outer tubing 202 can be a braided sheath. In some embodiments, the outer tubing 202 can also include a lining that fits around the outer tubing 202. In some embodiments, the lining can prevent air or other fluids to seep between the outer tubing 202. The outer tubing 202 can be coupled to the outer component 212. In some implementations, the surgical instrument 200 can be surrounded by a sheath or lining to avoid frictional contact between the outer surface of the outer tubing 202 and other surfaces. In some implementations, the surgical instrument 200 can be coated with Polytetrafluoroethylene (“PFTE”) to reduce frictional contact between the outer surface of the surgical instrument 200 and other surfaces, such as the inner wall of the subject.

The outer tubing 202 can be maneuvered within the subject. The insertion of the outer tubing 202 can be through the opening or the cavity. The outer tubing 202 can be turned, bent, or otherwise navigated through curvatures of the subject. For example, the outer tubing 202 can be maneuvered into a curved portion of the subject. The outer tubing 202 can be in contact with the subject, such that the outer tubing 202 can navigate through the curved portion of the subject. The outer tubing 202 can be bent or turned in response to reaching or being in contact with the curved portion, such that the outer tubing 202 curves through the curved portion while navigating. For example, the bodily cavity can include curves, bumps, or otherwise non linear paths to a treatment site. The treatment site can be located past the non-linear path within the subject. The outer tubing 202 can push, bump, or impact within the bodily cavity to turn through the non-linear path of the cavity. In some cases, the outer tubing 202 can be navigated through a cavity by bouncing, turning, or adjusting a navigation direction in response to contact with the cavity.

The outer tubing 202 can have a diameter less than 4 mm. The outer tubing 202 can be composed with higher or lower density, higher or lower malleability, higher or lower flexibility, or other features for ease of traversing through the subject. The flexibility of the outer tubing 202 facilitates the navigation of the surgical instrument 200 within the subject. The outer tubing 202 can be flexible as to not introduce injuries, tears, wounds, or other damages within the subject. The outer tubing 202 can include any width or length. The width can be 1 millimeter, 2 millimeters, 3 millimeter, 4 millimeters, 3 millimeters, or 1 centimeter. The length can be 350 mm, 500 mm, 1 meters, 2 meters, 3 meters, 4 meters, 5 meters, 6 meters, 7 meters, 8 meters, 9 meters, 10 meters, 50 meters, or 100 meters.

The outer tubing 202 can include or be coupled to one or more sensors, such as a light sensor, electromagnetic sensor, an optical stereotactic sensor, a pressure sensor, an impact sensor, a flow sensor, a radar sensor, a position sensor, or a distance sensor. In some embodiments, the outer tubing 202 detects a presence of the materials. The outer tubing 202 can be equipped with at least one sensor that can communicate with at least one external device, such as a sensor processing component (not shown) to determine the thickness of material relative to the rest of the subject indicated by the sensor. The sensor can include, for example, a temperature sensor, a pressure sensor, a resistance sensor, an impact sensor, an ultrasonic sensor, or other sensor for medical examination. In some embodiments, the type of material is associated with at lent an impedance or a density of the tissue. The sensor can gather temperature information and other sensed information, and provide signals corresponding to such information to the sensor-processing unit. The sensor-processing unit can subsequently identify the type of material. In some embodiments, the sensor can be an electrical sensor.

The outer tubing 202 can be an extendible and/or retractable wire. The extension of the outer tubing 202 can enable the cutting assembly 210 to move towards a treatment site within the subject. The cutting assembly 210 may extend or move pass the treatment site, in which an operator can terminate further extension of the outer tubing into the subject. While moving towards the treatment site, the operator may push or exert a form to the proximal end 204 of the outer tubing 202. The outer tubing 202 can be moved further inside the subject and towards the treatment site in response to the force exerted to the proximal end 204. The distal end tithe outer tubing 202 may be positioned a distance front the material, such as 1 millimeter, 1 inch, or 1 meter from the material.

The cutting assembly 210 can be coupled to or located at the distal end 206 of the surgical instrument 200. The cutting assembly 210 can be a distance from the distal end 206 of the outer tubing. For example, the distance can be 1, 2, 3, 4, 5, 6, 7, 8, 9, or 10 millimeters. The cutting assembly 210 can perform actions, including but not limited to, cutting, snaring, shredding, slicing, shattering, either entirely or partially, are also examples of debriding. Accordingly, the cutting assembly 210 may be a component that is capable of cutting, snaring, shredding, slicing, or shattering from a surface of the body of the subject. As such, the cutting assembly 210 may be implemented as a forceps, scissor, knife, snare, shredder, or any other component that can debride.

The cutting assembly 210 can include at least one sensor, such as proximity sensor, a light sensor, a pressure sensor, a radar sensor, a flow sensor, a flex sensor, an impact sensor, a distance sensor, or other sensor configured to inspect, examine, sense, or navigate through a body of a subject. The cutting assembly 210 may include a light source and a recording device or capturing device (e.g. a camera or a scope) to collect visual information from an inspective of the body of the subject. The light source can include a light emitting diode (“LED”), incandescent lamps, compact fluorescent, halogen, neon, or other types of lighting elements. The surgical instrument 200 or the cutting assembly may emit light and initiate recording using the light source and the recording device. The cutting assembly 210 may receive at least one visual information front the camera and transmit the at least one visual information to the display device. The display device can generate or display the images based on the received visual information for an operator or a doctor to view inside the body of the subject during an operation. In some embodiments, the cutting assembly 210 can be equipped with an injectable dye component through which the operator can use to determine the extent of narrowing under fluoroscopic guidance or to mark a particular region within the subject. In other embodiments, the operator can mark a particular region with the cutting assembly 210, without the use of an injectable dye.

The cutting assembly 210 can include the outer component 212 and the inner component 214 disposed within the outer component 212. The outer component 212 can be configured to pass fluid. The outer component 212 can be a component, cover, an outer tube, a shell, or a main body of the cutting assembly 210. The distal end 206 of the outer tubing 202 can be coupled to the outer component 212. The outer component 212 can be shaped or formed to, for example, a cylinder, a prism, a cone, or otter shapes. The outer component 212 can be flexible. The outer component 212 can bend and flex to any degree. In some embodiments, the outer component 212 can bend and flex to 10, 20, 30, 40, 50, 60, 70, 80, 90, 100, 110, 120,130, 140, 150, 160, 170, or 180 degrees. The outer component 212 can include a thickness. The thickness can be 10 nanometers, 20 nanometers, 1 millimeter, 2 millimeters, 3 millimeter, 4 millimeters, or 5 millimeters. The outer component 212 can include a width. The width can be 1 millimeter, 2 millimeter, 3 millimeter, 4 millimeters, 5 millimeters, or 1 centimeter. The outer component 212 can include a lend. The length can be 1 meters, 2 meters, 3 meters, 4 meters, 5 meters, 6 meters, 7 meters, 8 meters, 9 meters, 10 meters, 50 meters, 100 meters, etc. The outer component 212 can include a cross-sectional area, such as 0.6 millimeters squared, 1 millimeters squared, 1.9 millimeters squared, etc. The outer component 212 can be composed of materials, such as metal, steel, plastic, tuber, glass, carbon fiber, titanium, aluminum, or other alloys.

The outer component 212 can at least partially surround the inner component 214. In some embodiments, the inner component 214 cut any material suctioned into or otherwise entering the outer component 212. The outer component 212 can be a component, cover, a tube, or a shell. The inner component 214 can include an opening such that material cut by the cutting assembly 210 enters via the opening. The inner component 214 can include a length similar to or leas than the outer component 212. The length can be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 32, 53, 54, 55, 56, 57, 58, 59, 60, 61, 62, 63, 64, 65, 66, 67, 68, 69, 70, 71, 72, 73, 74, 73, 76, 77, 78, 80, 81, 82, 83, 84, 85, 86, 87, 88, 89, 40, 91, 92, 93, 94, 95, 96, 97, 98, 99, or 100 cm. The inner component 214 can be designed to facilitate debriding one or more materials and removing the cut materials in a single operation. The inner component 214 can be disposed within the outer component 212. The inner component 214 can couple with the outer component 212. The inner component 214 can be composed of a similar material as the outer component 212. The inner component 214 can be flexible, similar to the outer component 212.

The outer component 212 can define the cutting window 216. The outer component 212 can define the cutting window 216, at a distal end of the cutting assembly 210. A portion of the radial wall of the outer component 212 can define the cutting window 216 that extends around a portion of the radius of the outer component 212. In some embodiments, the operator can receive or retrieve the cut materials through the cutting window 216.

The cutting window 216 can be configured to enable the cutting assembly 210 to cut, dissect, or debride the material. For example, the cutting assembly 210 can initiate the debriding or cutting process by rotating the cutting through the material to receive the material in the cutting window 216. The cutting window 216 can positioned at a side of the cutting assembly 210. The cutting window 216 can be configured to enable tangential or side cutting of material with respect to the movement of the cutting assembly 210. In some embodiments, the outer component 212 can define the coning window 216. The cutting window 216 can include a hollow structure with a shape, such as a circle, an oval, a rectangle, or other geometric shape for exposing the blades of the cutting assembly 210. The cutting window 216 can include a diameter. The diameter can be 1 millimeter, 2 millimeters, 3 millimeters, 4 millimeters, or 5 millimeters. The cutting window 216 can include a cut out, which can be a portion of the cutting assembly 210. For example, the coning window 216 can include a 0.4 millimeters cut out.

The cutting assembly 210 can be configured to cut material froth a subject. The cutting ably 210 can include the blade (or a fan blade). The cutting assembly 210 can include one or more cutting members, such as a fan, an axial cutter, a drill, a hook, a scoop, a reamer, a miller cutter, or other cutting tools or devices. The cutting assembly 210 can be refers to as a debriding component, a cutter, a removal tool, or an extractor. The cutting assembly 210 can include a blade. The cutting members can be composed of one or more materials for cutting or dissecting a material, such as a steels, plastics, carbon fibers, titanium, aluminums, metals, or other alloys for performing laparoscopy or hysteroscopy operations.

The cutting assembly 210 may be actuated such that the cutting assembly 210 may be operated through the translation of mechanical forces exerted by an operator or automatically actuated, using a turbine, a motor (e.g., electrical motor), or any other force generating component to actuate the debriding component. The outer tubing 202 can be configured to receive a torque (e.g., τ-proximal) at the proximal end 204 and transmit the torque to the distal end 206 to the outer component 212 (e.g., as τ-distal) to rotate the outer component 212 to actuate the cutting assembly 210. The cutting assembly 210 can be configured to cut at various speeds, such as 5000 rotation per minute (“RPM”), 10,000 RPM, 20,000 RPM, or 50,000 RPM. The cutting assembly 210 may be manually operated or may utilize any other means of debriding material such that the cut material are capable of being retrieved from the treatment site via the outer tubing 202. The cutting assembly 210 can cut the material into small enough pieces, which may be retrieved via the surgical instrument 200 such that the surgical instrument 200 does not need to be cut from the subject to collect the cur material. It should be appreciated that the cutting assembly 210 is able to rotate a specific degree and with, a specific torque, equivalent or matching the rotation and torque of the motor or operator. Accordingly, the cutting assembly 210 can provide cutting precision, control and power consumption. For example, the cutting assembly 210, coupled to the cutting assembly 210, can rotate a number of degrees with a specific toque equivalent to an operator providing the degrees and torque to the motor. For instance, the operator or motor may initiate a 30-degrees rotation. The rotation, force, and tome can be exerted from the motor to the cutting assembly 210. The cutting assembly 210 can receive the exerted rotation. Accordingly, the rutting assembly 210 may rotate 30-degrees based on the exerted rotation, force, and torque of the motor or operator.

Referring to FIG. 2B in conjunction with FIG. 2C, the tubing sheath 218 of the surgical instrument 200 can encapsulate the outer tubing 202. The tubing sheath 218 can include laminate or heat shrink. The tubing sheath 218 can provide an additional shape, texture, groove, or other features to the outer tubing 202. The tubing sheath 218 can have any length. For example, the length can be 100, 200, 350, 500, 750, or 900 mm. The length of the outer tubing 202 can be sized to exceed the length of the tubing sheath 218. The outer tubing 202 can extend any distance past a distal end of the tubing sheath 218. For example, the outer tubing 202 can extend 10, 20, 30, 40, 50, 60, 70, 80, 90, or 100 mm past the distal end of the tubing sheath 218. The surgical instrument 200 can be sized, shaped, or configured such that the diameter is less than the diameter of the channel in which the surgical instilment 200 is to be instead.

The tubing sheath 218 and the outer tubing 202 can define a wire channel 219 (e.g., wire tunnel) configured to receive, maintain, or house the articulation wires 220. The tubing sheath 218 can be an external sheath that encloses or encapsulates the articulation wires 220. The wire channel 219 can extend from the proximal end 204 of the outer tubing 202 to the distal end 206 of the outer tubing 202. The tubing sheath 218 and the articulation member 209 can partially define the wire channel 219 such that the wire channel 219 and the articulation wires 220 disposed therein can extend along the articulation member 209.

The articulation wires 220 can be threads or wires that extend from the proximal end 204 to the distal end 206 of the outer tubing 202. The articulation wires 220 may be made from stainless steel (e.g. type 304V hard tempered stainless steel).

While shown is one articulation wire 220, it is contemplated that the outer tubing 202 can include any number of articulation wires 220, such as for example, 1, 2, 3, 4, 5, 8, 10, 24, 30, or 50 wires. It will be appreciated that the desired or optimal number of articulation wires 220 can depend on material properties of the articulation wires 220, spacing between the articulation wires 220, and other properties. In some implementations, the number of the articulation wires 220 can be selected for transmitting torque to the outer component 212.

The number of articulation wires 220 may be such that a percentage difference between articulation of proximal end 204 and the distal end 206 (and/or a percentage difference between an expected articulation of the distal end 206 and an actual articulation of the distal end 206, where the expected articulation is consistent with a smooth and/or proportional rotation response as discussed above) may be less than a threshold difference. In some implementations, the threshold difference is less than or equal to thirty percent. In some implementations, the threshold difference is less than or equal to twenty percent. In some implementations, the threshold difference is less than or equal to ten percent. In some implementations, the threshold difference is less than or equal to five percent. In some implementations, a measure of a ratio of τ-proximal to τ-distal as a function of τ-proximal (e.g., over a predetermined range of τ-proximal values) can be used to represent the performance of the outer tubing 202; for example, a ratio of the standard deviation of the ratio to the average value of the ratio can be less than a threshold ratio (which can indicate how constant the ratio of τ-proximal to τ-distal is as a function of τ-proximal). The threshold ratio can be less than or equal to 0.3. The threshold ratio can be less than or equal to 0.2. The threshold ratio can be less than or equal to 0.1. The threshold ratio can be less than or equal to 0.05.

Referring to FIGS. 2B-2D, the articulation wires 220 can be coupled to wire couplers 222 in the wire channel 219. The wire couplers 222 can be configured to secure the articulation wires 220 under tension. The wire couplers 222 can be coupled to or extended from the outer tubing 202. The wire couplers 222 can be located at the distal end 206 of the outer tubing 202. One or more articulation wires 220 within a wire channel 219 can be coupled to a respective wire coupler 222 in the wire channel 219. For example, two articulation wires 220 can be coupled to one wire coupler 222. In some embodiments, the wire couplers 222 can be disposed at different locations along the longitudinal axis 208. For example, a first wire coupler 222 can be disposed closest to the cutting assembly 210, while a second wire coupler 222 can be disposed in the middle of the articulation member 209 (e.g., closer to the proximal end 204). Articulation wires 220 connected to the first wire coupler 222 can cause a different articulation of the articulation member 209 than the articulation wires 220 connected to the second wire coupler 222.

Referring to FIG. 2D, shown is a cross sectional tunnel view of the articulation member 209, the tubing sheath 218, the wire channel 219, and the articulation wire 320. The articulation member 209 can have a diameter less than 4 mm. The articulation member 209 and the tubing sheath 218 can have a diameter that is together less than 4 mm.

The outer tubing 202, the articulation member 209, or the articulation wires 220 can include at least one of an elastomer or a friction reducing additive. In some implementations, the elastomer includes a thermoplastic elastomer such as polyether block amide (e.g., PEBAX). In some implementations, the friction reducing additive includes MOBILIZE, manufactured by Compounding Solutions, LLC of Lewiston, ME. The at least one of the elastomer or the friction reducing additive can reduce the likelihood of the articulation wires 220 sticking to the outer tubing 202, becoming kinked, or otherwise undergoing frictional losses. In addition, the at least one of the elastomer or the friction reducing additive can reduce friction generated between the outer tubing 202 and the outer component 212 when the outer tubing 202 and the outer component 212 come in contact with one another, for instance, when the surgical instrument 200 has been passed through a tortuous pathway. In some embodiments, the at least one of the elastomer or the friction reducing additive can reduce friction generated between an outer wall of the outer tubing 202 and an inner wall of the tubing sheath 218.

The surgical instrument 200 can include the handle 224. The handle 224 can be configured to be grasped by an operator of the surgical instrument 200. The handle 224 can include rubber, plastic, or any non-slip materials suitable for a medical environment. The handle 224 can include both the rotation actuator 226 and the articulation actuator 228. The handle 224 can be configured to enable the operator of the surgical instrument 200 to maneuver the surgical instrument 200, rotate the rotation actuator 226, and articulate the articulation actuator 228. For example, the handle 224 can be configured in a form factor to receive inputs to rotate the rotation actuator 226 and articulate the articulation actuator 228.

The surgical instrument 200 can include the rotation actuator 226 for articulating the articulation member 209 of the outer tubing 202 about the longitudinal axis 208 extending through the surgical instrument 200 responsive to receiving a first control input at the rotation actuator 226. The rotation actuator 226 can be coupled to the proximal end 204 of the outer tubing 202. The rotation actuator 226 can be coupled to the articulation wires 220. The rotation actuator 226 can be a knob, tube, handle, grip, or any other surface configured to receive control inputs from the operator. The control inputs can cause the rotation actuator 226 to rotate the proximal end 204 of the outer liking 202 about to the longitudinal axis 208. The control inputs can cause the rotation actuator 226 to pull on the articulation wires 220 to rotate the outer tubing 202 about to the longitudinal axis 208. For example, the control inputs can rotate the proximal end 204 of the outer tubing 202 by −90, −80, −70, −60, −50, −40, −30, −20, −10, 10, 20, 30, 40, 50, 60, 70, 80, or 90 degrees about the longitudinal axis 208.

The rotation actuator 226 can be configured to rotate the articulation member 209 about the longitudinal axis 208. The articulation member 209 can rotate at an angle proportional to the angle rotated by the rotation actuator 226. For example, the outer tubing 202 can be configured to rotate the articulation member 209 by 10 degrees responsive to a 10-degree rotation of the proximal end 204 by the rotation actuator 226. In another example, the articulation member 209 is configured to rotate according to any other configuration. For example, the articulation member 209 can be configured to rotate the articulation member 209 by 10 degrees responsive to a 10-degree natation of the rotation actuator 226, and rotate the articulation member 209 by 15 degrees responsive to a 20-degree rotation of the rotation actuator 226. In another example, the articulation member 209 can be configured to rotate the articulation member 209 by 10 degrees responsive to a 10-degree rotation of rotation actuator 226, and rotate the articulation member 209 by 25 degrees responsive to a 20-degree rotation of the rotation actuator 226.

The surgical instrument 200 can include the articulation actuator 228 coupled to the proximal end 204 of the outer tubing 202 and configured to articulate the articulation member 209 away from the longitudinal axis 208. For example, the articulation actuator 228 can articulate the articulation member 209 along the articulation axis 230. The articulation axis 230 can be relative to the longitudinal axis 208. For example, the articulation axis 230 can be −90, −80, −70, −60, −50, −40, −30, −20, −10, 10, 20, 30, 40, 50, 60, 70, 80, or 90 degrees relative to the longitudinal axis 208.

Articulation of the articulation member 209 can enable the cutting assembly 210 to more effectively reach remote sample sites while reducing risk of damage to or reduced functionality of components of the surgical instrument. The cutting assembly 210 can be manipulated while the articulation member 209 is articulated (e.g., changed in orientation in one or more axes) or at different times. For example, the orientation can be manipulated in a first axis perpendicular to the longitudinal axis 208 (e.g., to move the articulation member 209 up or down relative to a frame of reference defined with respect to longitudinal axis 208, or a second axis (e.g., to move the articulation member 209 left or right relative to the frame of reference defined with respect to longitudinal axis 208). The orientation can be manipulated to change an orientation of a cutting window 216 of the cutting assembly 210. Articulation can enable the cutting assembly 210 to maneuver in a greater range of positions for reaching material at the site within the subject.

The articulation actuator 228 can be configured to receive control inputs from the operator. The articulation actuator 228 can be a slider mechanism, grip, or any other surface configured to receive control inputs. The control inputs can pull or actuate the articulation actuator 228. For example, the control inputs can pull or push the articulation actuator 228 along the longitudinal axis 208. The control inputs can pull or push the articulation actuator 228 any distance, such as 1, 2, 3, 4, 5, 6, 7, 8, 9, 10, 20, 34, 40, or 50 mm. The distance can be based on the length of the handle 224.

The articulation actuator 228 can be coupled to the articulation wires 220. When the articulation actuator 228 pulls or pushes on the articulation wires 220, the articulation wires 220 transmit the force from the articulation actuator 228 to articulate, bend, or straighten the articulation member 209. In some embodiments, the pushing or pulling force provided by the articulation actuator 228 creates tension in the articulation wires 220 to cause articulation of the articulation member 209. In some embodiments, the articulation wires 220 have threshold rigidity that corresponds to a nominal tension or maximum tension that can be applied to the articulation wires 220, such that the articulation member 209 complies with (e.g., can be compressed or otherwise modified in shape) tension applied by the articulation wires 220. For example, the pushing or pulling of the articulation wirers 220 by the articulation actuator 228 can cause the articulation member 209 to articulate 90, −80, −70, 40, −50, −40, −30, −20, −10, 10, 20, 30, 40, 50, 60, 70, 80, or 90 degrees around the articulation axis 230. The articulation member 209 can articulate at an angle proportional to the distance moved by the articulation actuator 228. For example, the articulation actuator 228 can be configured such that a first force causes a 30-degree articulation of the articulation member 209, and a second force causes a 60-degree articulation of the articulation member 209. The second force can be stronger than the first force. For example, the first force can be 5 N. and the second force tan be 10 N.

The articulation actuator 228 can couple to or control the articulation wires 220 by using control mechanisms such as carbide clamps, guitar mechanisms, or tensioning rods (e.g., truss rods). In some embodiments, the tensioning rod is disposed along the outer tubing 202. The tensioning rod is configured to maintain a tension of the articulation wires 220 to control the articulation member 209 coupled to the cutting assembly 210. The control mechanisms can regulate the articulation of the articulation member 209. For example, the operator can configure the control mechanisms such that articulation member 209 articulates by 10 degrees responsive to a 5 mm pull of the articulation actuator 228. In another example, the articulation member 209 is configured to articulate according to any other configuration. For example, the articulation member 209 can be configured to articulate by 10 degrees responsive to a 5 mm pull by the articulation actuator 228, and articulate by 15 degrees responsive to a 10 mm pull by the articulation actuator 228. In another example, the articulation member 209 can be configured to articulate by 10 degrees responsive to a 5 mm pull by the articulation actuator 228, and articulate by 25 degrees responsive to a 10 mm pull by the articulation actuator 228.

In some embodiments, the surgical instrument 200, the natation actuator 226, or the articulation actuator 228 can include a locking mechanism. The locking mechanism can restrict movement of the articulation member 209 to set the articulation member 209 to a target orientation. In some embodiments, the locking mechanism is a knob, switch, or any other satiate configured to receive control inputs from the operator. In some embodiments, the locking mechanism can be operated to selectively restrict movement of the articulation member 209 to a single degree of freedom (e.g., lock movement of the articulation member 209 in the first axis while allowing movement in the second axis). In some embodiments, the locking mechanism is activated responsive to actuation or rotation of the rotation actuator 226. In some embodiments, the locking mechanism is activated responsive to actuation of the cutting assembly 210 to cut the material. In some embodiments, the locking mechanism is activated responsive to rotation of a flexible torque component 232 to rotate the inner component 214 relative to the outer component 212 to cut the material. In some embodiments, the locking mechanism is activated responsive to suction provided by a vacuum source to retrieve the cut material. The locking mechanism can include one or mote clamps, gears, brakes, or any combination thereof that can selectively contact or apply tension to tee or more of the articulation wires 220 to prevent the selected one or more control members from moving. The locking mechanism can be configured to spool or unspool the articulation wires 220.

The surgical instrument 200 can include the flexible torque component 232 disposed within the outer tubing 202. The flexible torque component 232 can be coupled to and disposed within the inner component 214. In addition, at least one of the a elastomer or the friction reducing additive can reduce friction generated between the flexible torque component 232 and the inner component 214 when the flexible torque component 232 and the inner component 214 come in contact with one another, for instance, when the surgical instrument 200 has been passed through a tortuous pathway. The flexible torque component 232 can be configured to rotate the inner component 214 relative to the outer component 212 to cut the material. The flexible torque component 232 can be composed of at least one of metal, steal, plastic, titanium, nickel, carbon fiber, or other alloys. In some embodiments, the inner component 214 can include a lining within which the flexible torque component 232 is disposed.

The flexibility of the outer tubing 202 can allow the surgical instrument 200 to rotate while being articulated, and articulate while being rotated. For example, the flexible tubing of the surgical instrument 200 may be articulated 120 degrees, including the components within the surgical instrument 200 such as the flexible torque component 232, and the surgical instrument 200 can maintain the rotational performance with the flexibility of the flexible tubing at the 120 degrees articulation.

The surgical instrument 200 can include an aspiration channel 234 extending from the cutting window 216 to the aspiration port 236. The aspiration channel 234 can be partially defined by the flexible torque component 232. The aspiration channel 234 can be partially defined by an outer well of the inner component 214. The aspiration channel 234 can be partially defined by an inner wall of the outer component 212. Materials can enter the aspiration channel 234 via the cutting window 216 and traverse the length of the aspiration channel 234 to the aspiration port 236.

The aspiration port 236 can be an opening or any other connection between the outer tubing 202 and the external tubing 238. The aspiration port 236 can include sockets, plugs, or any other coupling mechanism configured to couple the outer tubing 202 and the external tubing 238. The external tubing 238 can be coupled to a vacuum source configured to suction, retrieve, extract, or collect cut material from the aspiration channel 234. The external tubing 238 can be coupled to a motor configured to rotate the flexible torque component 232 to rotate the inner component 214 relative to the outer component 212. In some embodiments, the external tubing 238 can introduce irrigation fluid, such as a saline or water, into the surgical instrument 200, and the irrigation fluid can flow to the treatment site.

The surgical instrument 200 can include a light configured to illuminate the treatment site. The light can be a fiber optic light, a light emitting diode (“LED”), incandescent lamps, comfit fluorescent halogen, neon, or other types of lighting elements. In some embodiments, actuating the notion actuator 226 or the articulation actuator 228 can actuate the light to turn it on, off, or modulate its intensity.

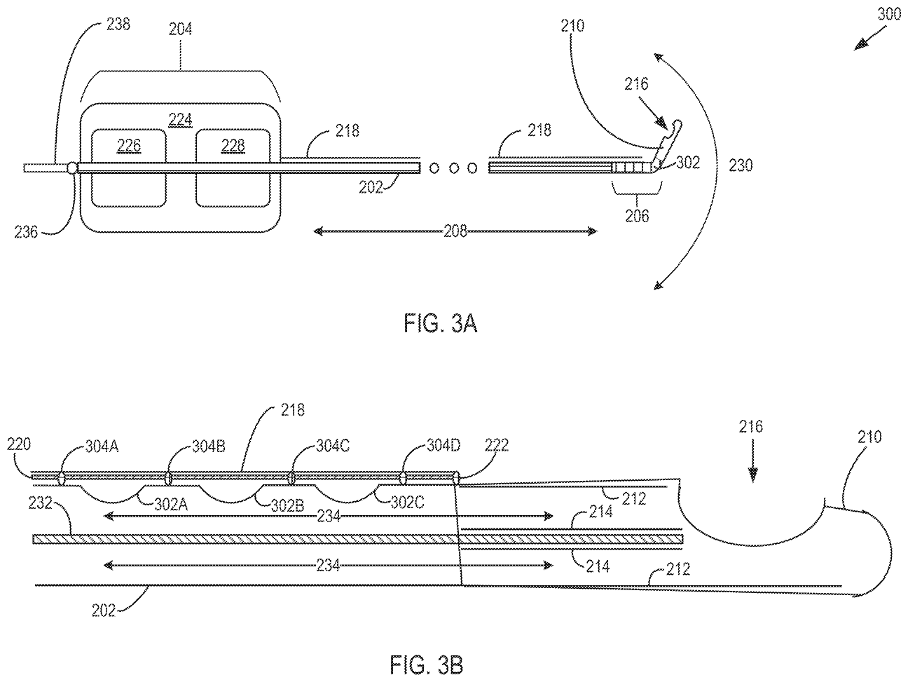

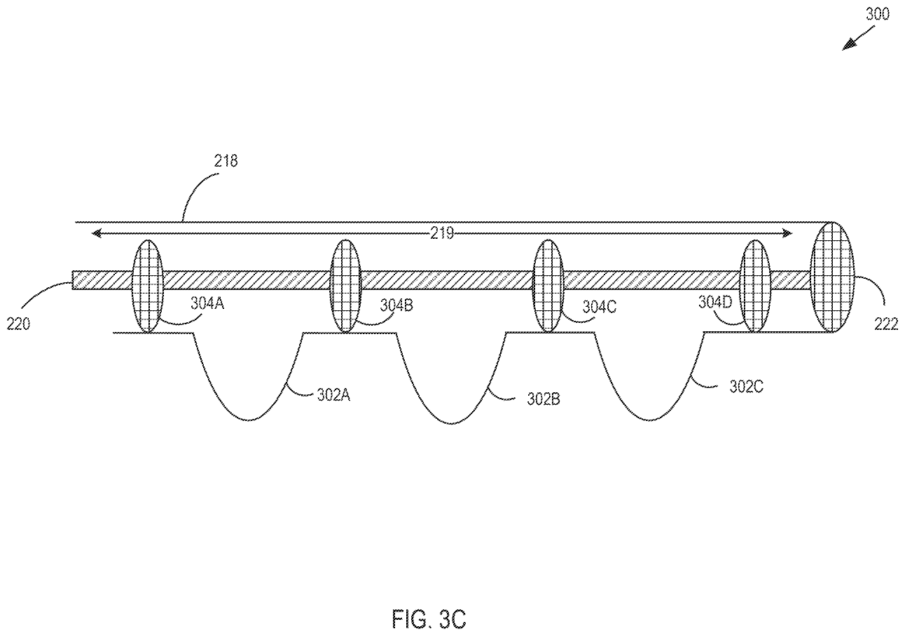

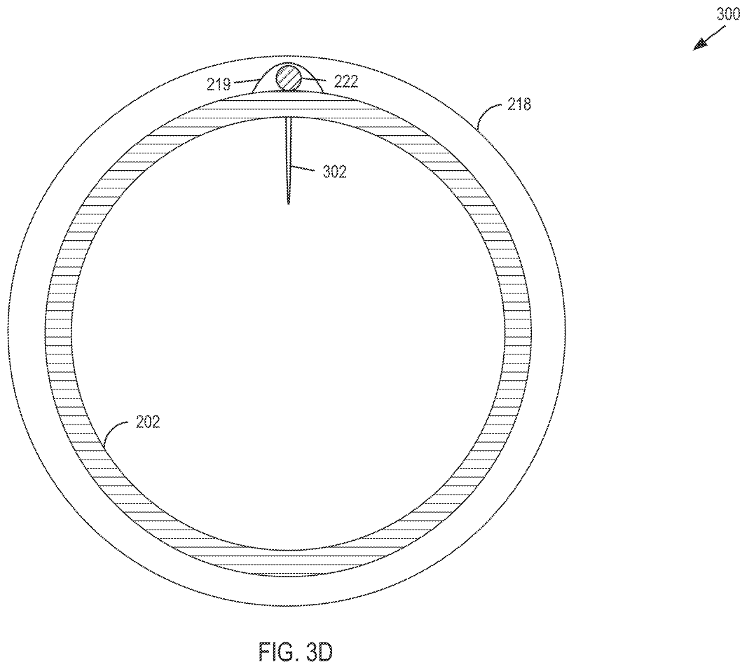

Referring to FIGS. 3A-3D, the surgical instrument 300 can be similar to, and can include the same structure and functionality as the surgical instrument 200, but differs in that the surgical immanent 300 can include segments 302A-302C (generally referred to as segments 302) instead of the articulation member 209, and the segments 302 can include securing elements 304A-304D (generally refereed to as securing elements 304) configured to secure the wires. The segments 302 are advantageous by being able to articulate independently of each other, which enables the operator of the surgical instrument 300 to precisely articulate the cutting window 216 to the treatment site. The securing elements 304 are advantageous by securing the articulation wires 220 along the segments 302 to enable more precise control over each individual segment 302. By enabling the individual articulation of each segment 302, the securing elements 304 allow for maneuvering of the cutting assembly 210 counted to at least one of the segments 302 to specific locations (e.g., material) while maintaining the rest of the surgical instrument 300 in position at the treatment site and/or the cavity of the subject. For example, the securing elements 304 closer to the proximal end 204 (e.g., securing element 304A) can lock or stiffen adjacent segments 302 (e.g., segment 302A) such that the distal segments (e.g., segment 302C) can articulate without articulating the proximal segments 302 (e.g., segment 302A), which can enable the operator of the surgical instrument 300 to more precisely articulate the cutting window 216 to the treatment site.

The segments 302 (also known as vertebrae) can be located at the distal end 206 of the outer tubing 202. The segments 302 can be configured to have additional malleability or flexibility relative to the outer tubing 202. For example, each of the segments 302 can be configured to independently bend. The additional malleability or flexibility of the segments 302 enables the articulation of the distal end 206 within the subject and along the longitudinal axis 208.

Referring to FIG. 3D, shown is a cross sectional tunnel view of the outer tubing 202, the tubing sheath 218, the wire channel 219, the articulation wire 220, and the segment 302. The outer tubing 202 can have a diameter less than 4 mm. The outer tubing 202 and the tubing sheath 218 can have a diameter that is together less than 4 mm.

The securing elements 304 can be configured to secure the articulation wires 220 under tension. The tubing sheath 218 can enclose the securing elements 304. The securing elements 304 can be disposed on or extended from the segments 302. The securing elements 304 can be grommet, rings, or edge strips. The securing elements can be made or metal, plastic, or rubber. The securing elements 304 can be flared or collared along the segments 302. The securing elements 304 can be disposed along the longitudinal axis 208 of the outer tubing 202. Each securing element 304 can correspond to a segment 302. For example securing element 304A can be disposed adjacent to the segment 302A. Sets of securing elements 304 can be disposed on different sides of the outer tubing 202. For example, a first set of securing elements 304A-304D can be disposed on the segments 302A-302C, and a second set of securing elements 304 can be disposed on a second side of the perimeter of the outer tubing 202, the second side positioned 180 degrees away and around the perimeter relative to the first side. Conversely, in another example, the surgical instrument 300 can have only segments 302 and corresponding securing elements 304 disposed on one side. For example, the surgical instrument 300 can have only segments 302A-302C and corresponding securing elements 304A-304D to secure the wire 220.

The rotation actuator 226 can be configured to articulate the segments 302 about the longitudinal axis 208 extending thresh the surgical instrument 200 responsive to receiving a first control input at the rotation actuator 226. The rotation actuator 226 can be coupled to the proximal end of the outer tubing 202. The rotation actuator 226 can be coupled to the articulation wires 220. The rotation actuator 226 can be a knob, tube, handle, grip, or any other surface configured to receive control inputs from the operator. The control inputs can cause the rotation actuator 226 to rotate the proximal end 204 of the outer tubing 202 about to the longitudinal axis 208. The control inputs can cause the rotation actuator 226 to pull on the articulation wires 220 to rotate the outer tubing 202 about to the longitudinal axis 208. For example, the control inputs can rotate the proximal end 204 of the outer tubing 202 by −90, −80, −70, −60, −50, −40, −30, −20, −10, 10, 20, 30, 40, 50, 60, 70, 80, or 90 degrees about the longitudinal axis 208.

The rotation actuator 226 can be configured to route the segments 302 about the longitudinal axis 208. The segments 302 can rotate at an angle proportional to the angle rotated by the rotation actuator 226. For example, the rotation actuator 226 can be configured to rotate the segments 302 by 10 degrees responsive to a 10-degree rotation of the proximal end 204 by the rotation actuator 226. In another example, the segments 302 is configured to rotate according to any other configuration. The securing elements 304 can be configured to stabilize or secure the articulation wires 220 along the segments 302 for precise control of the rotation. For example, the segments 302 can be configured to rotate the segments 302 by 10 degrees responsive to a 10-degree rotation of the rotation actuator 226, and rotate the segments 302 by 15 degrees responsive to a 20-degree rotation of the rotation actuator 226. In another example, the rotation actuator 226 can be configured to rotate the segments 302 by 10 degrees responsive to a 10-degree rotation of the rotation actuator 216, and rotate the segments 302 by 25 degrees responsive to a 20-degree rotation of the rotation actuator 226.

The articulation actuator 228 can be configured to articulate the segments 302 away from the longitudinal axis 208. For example, the articulation actuator 228 can articulate the segments 302 along the articulation axis 230. The articulation axis 230 can be relative to the longitudinal axis 208. For example, the articulation axis 230 can be −90, −80, −70, −60, −50, −40, −30, −20, −10, 10, 20, 30, 40, 50, 60, 70, 80, or 90 degrees relative to the longitudinal axis 208.

Articulation of the segments 302 can enable the cutting assembly 210 to more effectively reach remote sample sites while reducing risk of damage to or reduced functionality of components of the surgical instrument. The cutting assembly 210 can be manipulated while the segments 302 is articulated (e.g., changed in orientation in one or more axes) or at different times. For example, the orientation can be manipulated in a first axis perpendicular to the longitudinal axis 208 (e.g., to move the segments 302 up or down relative to a frame of reference defined with respect to longitudinal axis 208, or a second axis (e.g., to move the segments 302 left or right relative to the frame of reference defined with respect to longitudinal axis 208). The orientation can be manipulated to change an orientation of a cutting window 216 of the cutting assembly 210. Articulation can enable the cutting assembly 210 to maneuver in a greater range of positions for reaching material at the site within the subject.

When the articulation actuator 228 pulls or pushes on the articulation wires 220, the articulation wires 220 transmit the force from the articulation actuator 228 to articulate, bend, or straighten the segments 302. In some embodiments, the pushing or pulling force provided by the articulation actuator 228 creates tension in the articulation wires 220 to cause articulation of the segments 302. In some embodiments, the articulation wires 220 have threshold rigidity that corresponds to a nominal tension or maximum tension that can be applied to the articulation wires 220, such that the segments 302 complies with (e.g., can be compressed or otherwise modified in shape) tension applied by the articulation wires 220. For example, the pushing or pulling of the articulation wires 220 by the articulation actuator 228 can cause the segments 302 to articulate 90, −80, −70, −60, −50, −40, −30, −20, −10, 10, 20, 30, 40, 50, 60, 70, 80, or 90 degrees around the articulation axis 230. The segments 302 can articulate at an angle proportional to the distance moved by the articulation actuator 228. For example, the articulation actuator 228 can be conferred such that a first force causes a 30-degree articulation of the segments 302, and a second force causes a 60-degree articulation of the segments 302. The second force can be stronger than the first force. For example, the first force tan be 5 N. and the second force can be 10 N. The number of segments 302 that articulate can be proportional to the distance moved by the articulation actuator 228. For example, the articulation actuator 228 can be configured such that a first force causes articulation of the segments 302C that is closest to the wire couplers 222, and a second force articulation of both segments 302C and 302B. The second force can be stronger than the first force.

The wire couplers 222 can be disposed at different locations along the longitudinal axis 208 and adjacent to the segments 302 to enable selective articulation of the segments 302. For example, a first wire coupler 222 can be disposed adjacent to the segment 302C, while a second wire coupler 222 can be disposed adjacent to segment 302A (e.g., closer to the proximal end 204). Articulation wires 220 connected to the first wire coupler 222 can cause a different articulation of the segments 302 than articulation wires 220 connected to the second wire coupler 222. In some embodiments, the articulation actuator 228 coupled to the one or more articulation wires 220 can selectively articulate at least one of the plurality of segments 302 coupled to the coning assembly away from the longitudinal axis 208. For example, a first force applied to the articulation wires 220 connected to the first coupler 222 can cause articulation of the segments 302A-302C, but a second force applied to the articulation wires 220 connected to the second coupler 222 can cause articulation of the segment 302A.

The segments 302 and the securing elements 304 can include at least one of an elastomer or a friction reducing additive. In some implementations, the elastomer includes a thermoplastic elastomer such as polyether block amide (e.g., PEBAX). In some implementations, the friction reducing additive includes MOBILIZE, manufactured by Compounding Solutions, LLC of Lewiston, ME. The at least one of the elastomer or the friction reducing additive can reduce the likelihood of the segments 302 end the securing elements 304, becoming kinked, or otherwise undergoing frictional losses. In some embodiments, the at least one of the elastomer or the friction reducing additive can reduce friction generated between the segments 302 and the tubing sheath 218.

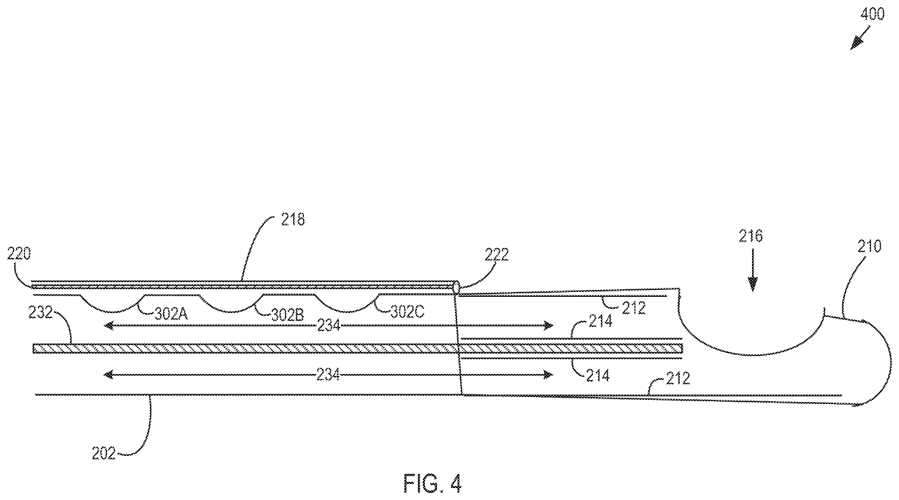

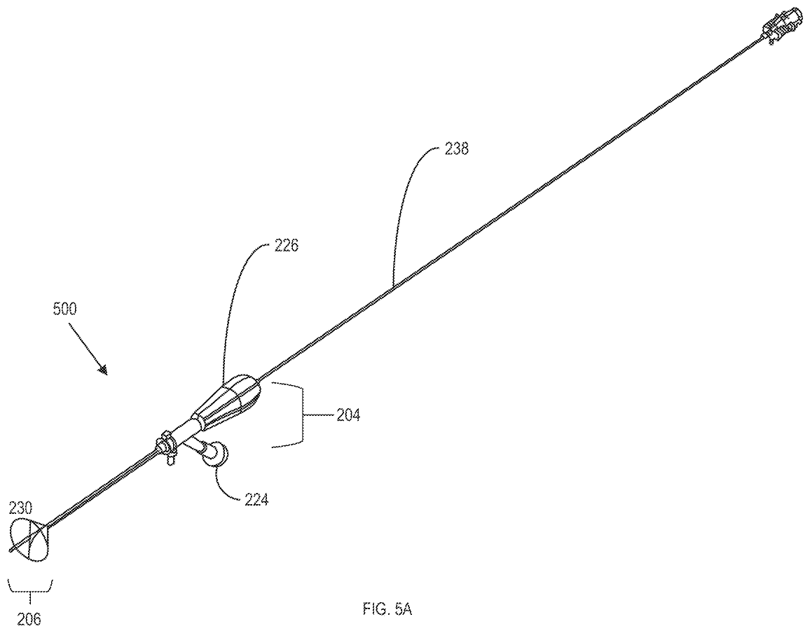

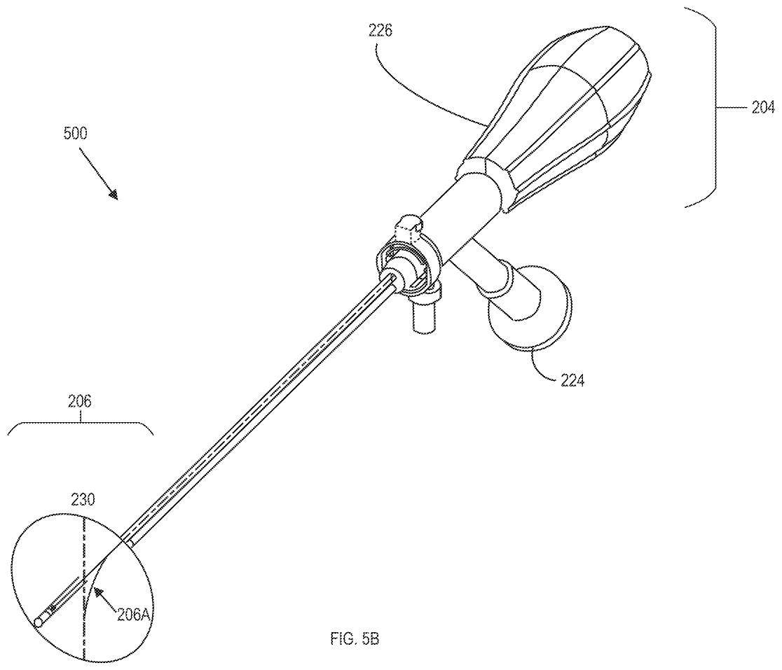

Rehiring to FIG. 4, the surgical instrument 400 can be similar to, and can include the same structure and functionality as the surgical instrument 300, but differs in that the surgical instrument 400 does not include securing elements 304. The surgical instrument 400 can be advantageous by enabling a single control input to articulate all the segments 302. The articulation wires 220 can also compress and retract inside the wire channel 219. For example, pulling of the articulation wire 220 coupled to the wire couplers 222 nearest the distal segments 302 (e.g., segment 302C), causes some articulation (but not necessarily the same) of all the segments 302A-302C, which can enable the operator of the surgical instrument 400 to articulate the cutting window 216 to the treatment site.

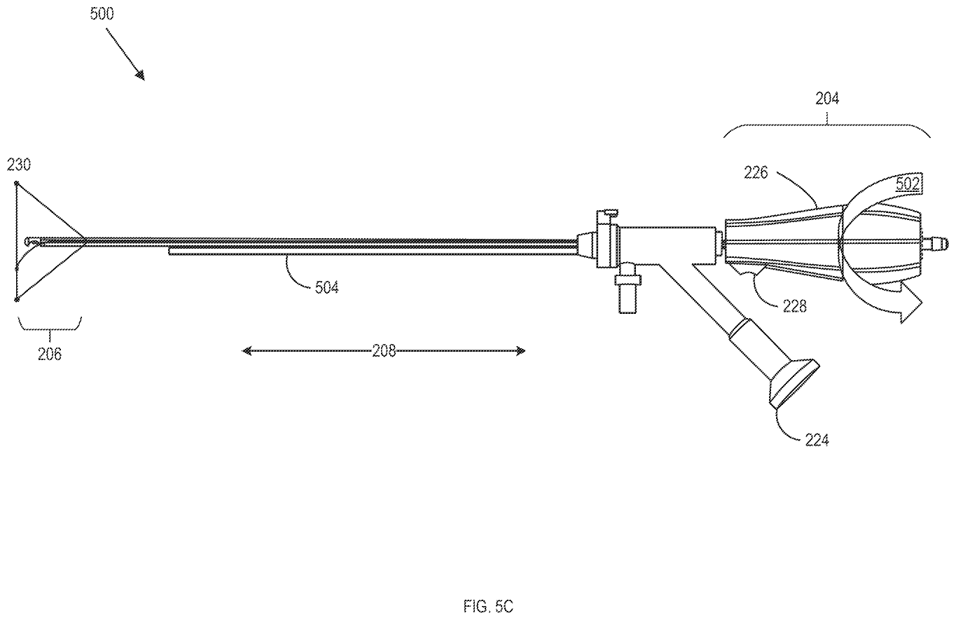

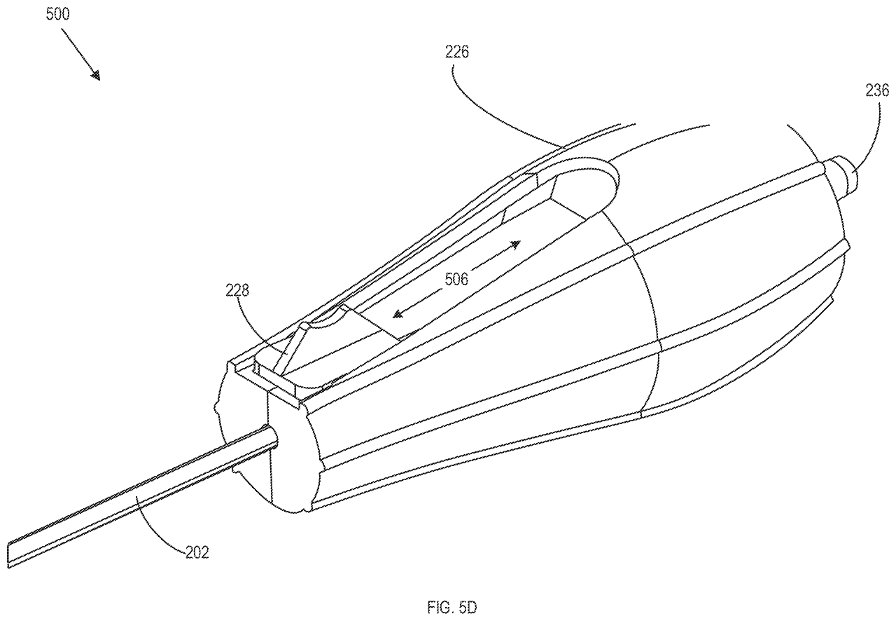

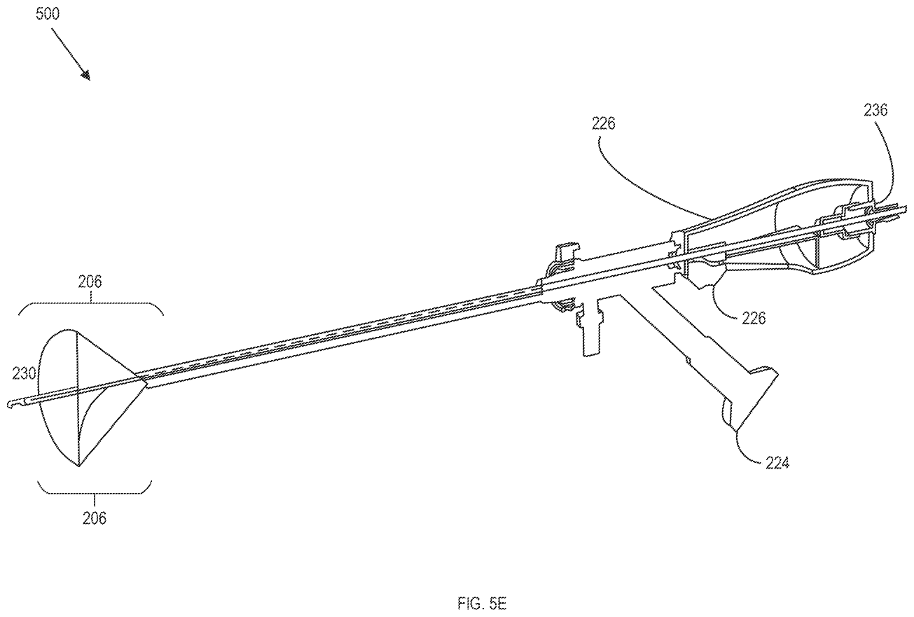

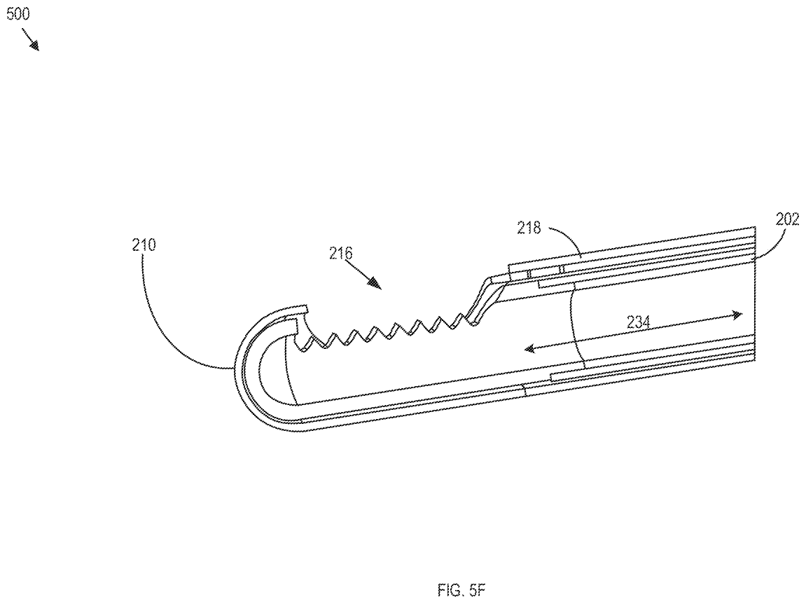

Referring to FIGS. 5A-5F, shown are perspective views of a surgical instrument 500 for maneuvering to a treatment site during a laparoscopic or hysteroscopic procedure according to embodiments of the present disclosure. The surgical instrument 500 can be similar to, and can include the same structure and functionality as the surgical instrument 200, 300 or 400.

Referring to FIG. 5A, shown is a perspective view of a surgical instrument 500 according to embodiments of the present disclosure. The operator can couple the surgical instrument 500 to the external tubing 238 via the aspiration port 236. The operator can receive irrigation fluid or aspirate cut material via the aspiration port 236 from the external tubing 238.

Referring to FIG. 5B, shown is a perspective view of the surgical instrument 500 according to embodiments of the print disclosure. The distal end 206 can be inserted, situated, or resided in the subject through an opening or a cavity. The articulated distal end 206 can be articulated around the articulation axis 230.

Referring to FIG. 5C, shown is a side view of the surgical instrument 500 according to embodiments of the present disclosure. The operator can actuate or rotate the rotation actuator 226 along the rotational axis 502 to rotate the distal end 206 about the longitudinal axis 208. The surgical instrument 500 can include an instrument tube 504, which can be a rigid tube extending from the proximal end 204. The operator can actuate the articulation actuator 228 to articulate the distal end 206 around the articulation axis 230. The articulated distal end 206A can be articulated around the articulation axis 230.

Referring to FIG. 5D, shown is a perspective view of the rotation actuator 226 and the articulation actuator 228 of the surgical instrument 500 for maneuvering to a treatment site during a laparoscopic or hysteroscopic procedure according to embodiments of the present disclosure. The rotation actuator 226 can be configured to cause the outer tubing 202 to notate. The articulation actuator 228 can be coupled to the articulation wire 220A. The articulation actuator 228 can be configured to slide across the control axis 506. The articulation actuator 228 can be configured to pull the articulation wire 220 to articulate the distal end 206 of the outer tubing 202.

Referring to FIG. 5E, shown is a case sectional side view of the surgical instrument 500 according to embodiments of the present disclosure. The articulated distal end 206 can be articulated around the articulation axis 230. The aspiration port 236 can couple the surgical instrument 200 to the aspiration pan 236.

Referring to FIG. 5F, shown is a cross sectional view of the cutting assembly 210 of the surgical instrument 200 according to embodiments of the present disclosure. The cutting assembly 210 can include the cutting window 216. The cutting assembly 210 can cut material, which can aspirate via the aspiration channel 234.

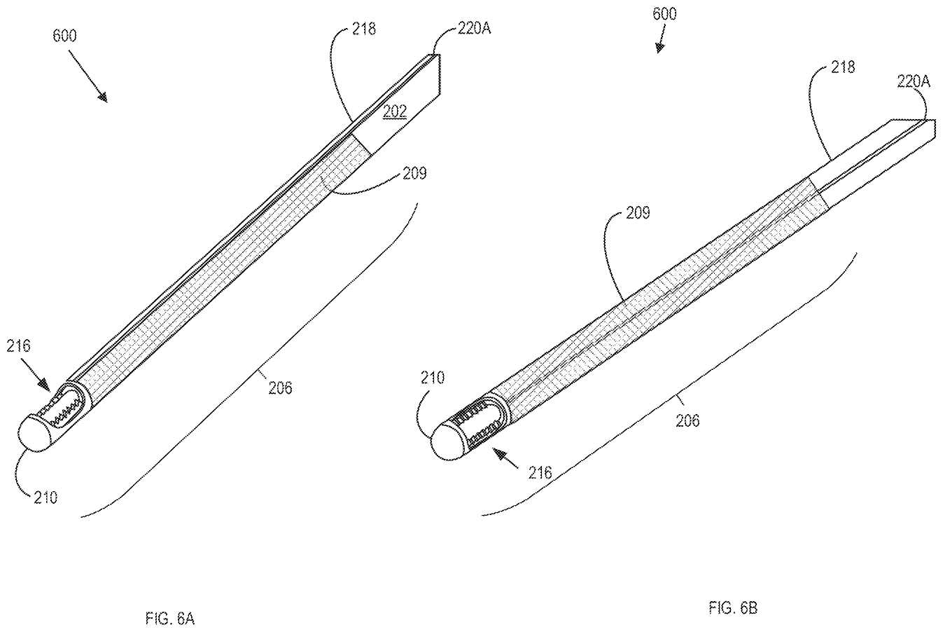

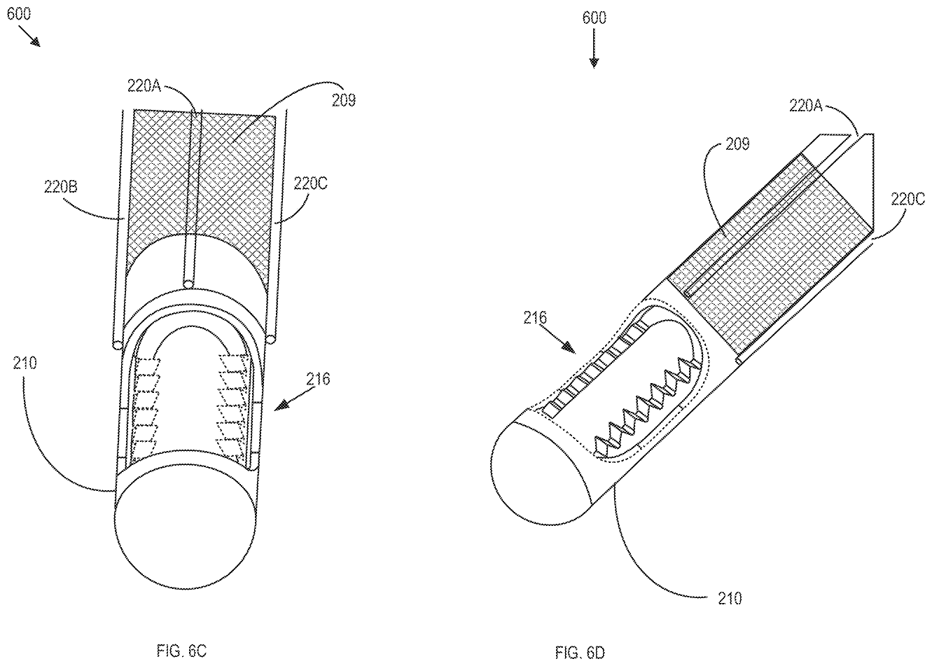

Retrying to FIGS. 6A-6D, shown are perspective views of a surgical instrument 600 for maneuvering to a treatment site during a laparoscopic or hysteroscopic procedure according to embodiments of the present disclosure. The surgical instrument 600 can be similar to, and can include the same structure and functionality as the surgical instrument 200 or 500.

Referring to FIGS. 6A and 6B, shown is a perspective view of the outer tubing 202 of the surgical instrument 600. The articulation member 209 can be coupled to the cutting assembly 210. The cutting assembly 210 can define the cutting window 216. The articulation wire 220A can be configured to articulate the articulation member 209 of the outer tubing 202.

Referring to FIGS. 6C and 6D, shown is a perspective view of the cutting assembly 210 of the surgical instrument 600 that include the articulation wires 220A-220C. The cutting assembly 210 can include the cutting window 216. The cutting assembly 210 can be coupled to the outer tubing 202.

The articulation wires 220 can be configured to articulate the ductal end 206 of the outer tubing 202. In certain embodiments, the articulation wires 220 art coupled to the distal end 206 of the outer tubing 202. The articulation wires 220 can be coupled to the rotation actuator 226, a gear, or other linear-motion to rotational motion cavorter such that upon pulling the articulation wire 220A, the distal end 206 of the outer tubing 202 would articulate in a first direction. For example, the articulation wire 220A can be configured to receive a force that pulls the articulation wire 220A. The force can apply tension to the articulation wire 220A towards the proximal end 204 of the articulation actuator 228. The tense articulation wire 220A can articulate the distal end 206. For example, the pulled articulation wire 220A can pull the distal end 206 towards the proximal end 204. Similarly, the articulation wire 220B can be coupled to another gear or other linear-motion to rotational motion convener such that pulling the articulation wire 220B could rain the distal end 206 of the outer tubing 202 coupled to the gear to articulate in a second direction opposite the first direction. The amount of linear force applied to the articulation wires 220 can determine an amount the outer tubing 202 can articulate.

The articulations wires 220 can be configured to be disposed at equal distances or angles relative to each other. For example, the articulation wire 220A can be disposed 90 degrees away from articulation wire 220C about the perimeter of the outer tubing 202, which itself is disposed 90 degrees from the articulation wire 220B about the perimeter of the outer tubing 202.

Applying tension to one or more of the articulation wires 220 can selectively articulate the articulation member 209 in a direction corresponding to how the one or more articulation wires 220 are disposed. As such, the articulation wires 220 can be configured to articulate the articulation member 209 along multiples axes. Additional articulation actuators 228 can couple to articulation wires 220 corresponding to articulation in other directions. For example, a first articulation actuator 228 can be coupled to the articulation wire 220A configured to cane the articulation member 209 to articulate along an axis of the articulation wire 220A, a second articulation actuator 228 can be coupled to the articulation wire 220B configured to cause the articulation member 209 to articulate along an axis of the articulation wire 220B, and a third articulation actuator 228 can be coupled to the articulation wire 220C configured to cause the articulation member 209 to articulate along an axis of the articulation wire 220C. In particular, if the articulation wire 220B is disposed on a flat side of the outer tubing and the articulation wire 220C is disposed on a wand side of the outer tubing, then the flat articulation actuator 228 can be configured to cause articulation of the distal end along the first side and the second articulation actuator 228 can be configured to cause articulation of the distal end along the second side.

Referring to FIGS. is 7A-7E, shown are perspective views of a surgical instrument 700 for maneuvering to a treatment site during a laparoscopic or hysteroscopic procedure according to embodiments of the present disclosure. The surgical instrument 700 can be similar to, and can include the same structure and functionality as the surgical instrument 300 or 500.

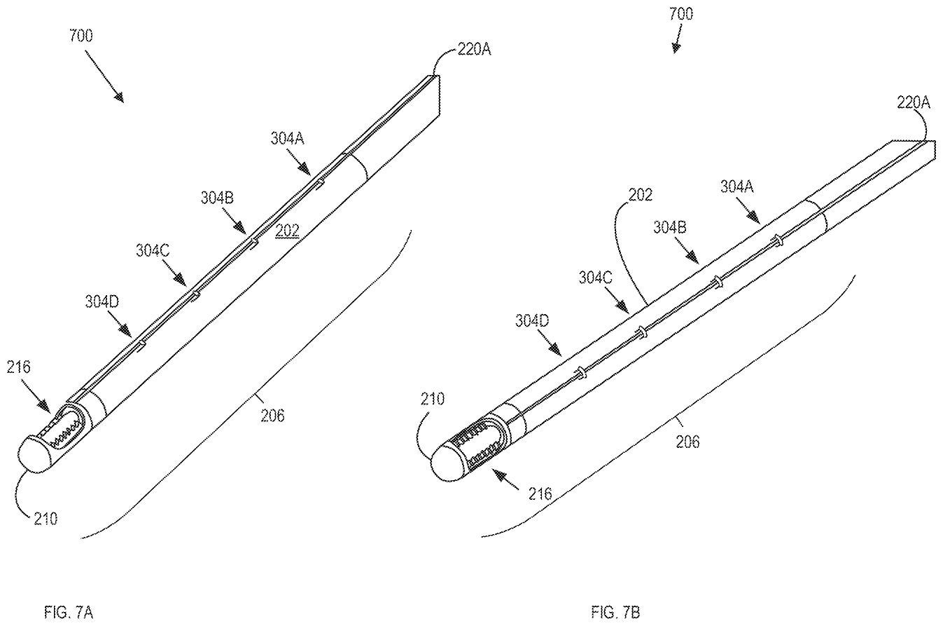

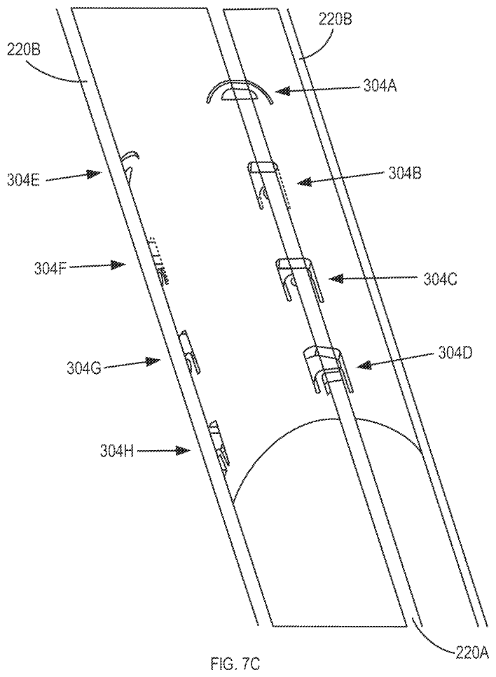

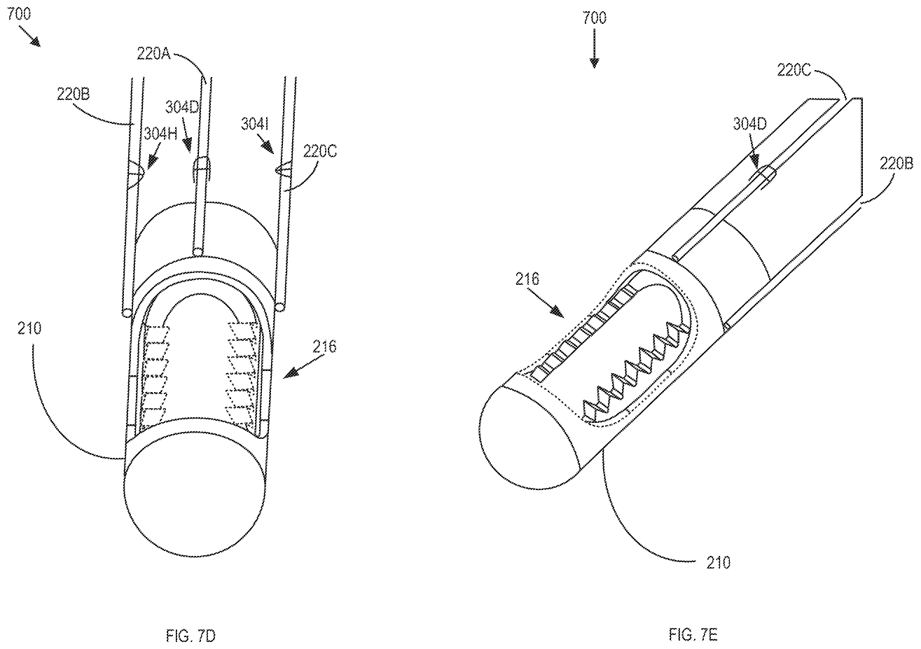

Referring to FIGS. 7A and 7B, shown is a perspective view of the outer tubing 202 of the surgical instrument 700 according to embodiments of the present disclosure. The outer tubing 202 can include the securing elements 304A-304D configured to secure the articulation wire 220A. The cutting assembly 210 can define the cutting window 216. The articulation wire 220 can be configured to articulate the distal end 206 of the outer tubing 202. The distal end 206 can include the segments 302 (not shown).

Referring to FIG. 7C, shown is a perspective view of the outer tubing 202 of the surgical instrument 700 according to embodiments of the present disclosure. The outer tubing 202 can include the securing elements 304A-304D configured to secure the articulation wire 220A. The outer tubing 202 an include the securing elements 304E-304H configured to secure the articulation wire 220B.

Referring to FIGS. 7D and 7E, shown is a perspective view of the cutting assembly 210 of the surgical instrument 700 according to embodiments of the present disclosure. The surgical instrument 700 can include the articulation wires 220A-220C. The outer tubing 202 can include the wring elements 304D, 304H, and 304I configured to secure the articulation wires 220A, 220B, and 220C, respectively.

The articulation wires 220 can be configured to articulate the distal end 206 (that include the segments 302) of the outer tubing 202. In certain enrolments, the articulation wires 220 are coupled to the distal end 206 of the outer tubing 202. The articulation wires 220 can be coupled to the rotation actuator 226, a gear, or other linear-motion to rotational motion convener such that upon pulling the articulation wire 220A, the distal end 206 of the outer tubing 202 would articulate in a first direction. For example, the articulation wire 220A can be configured to receive a force that pulls the articulation wire 220A. The force can apply tension to the articulation wire 220A towards the proximal end 204 of the articulation actuator 228. The tense articulation wire 220A can articulate the distal end 206. For example, the pulled articulation wire 220A can pull the distal end 206 towards the proximal end 204. Similarly, the articulation wire 220B can be coupled to another gear or other linear-motion to rotational motion convener such that pulling the articulation wire 220B could cause the distal end 206 of the outer tubing 202 coupled to the gear to articulate in a second direction opposite the first direction. The amount of linear force applied to the articulation wires 220 can determine an amount the outer tubing 202 can articulate.

The articulation wires 220 can be configured to be disposed at equal distances or angles relative to each other. For example, the articulation wire 220A can be disposed 90 degrees away from articulation wire 220C about the perimeter of the outer tubing 202, which itself is disposed 180 degrees from the articulation wire 220B about the perimeter of the outer tubing 202.

Applying tension to one or more of the articulation wires 220 can selectively articulate the articulation member 209 in a dilution corresponding to how the one or more articulation wires 220 are disposed. As such, the articulation wires 220 can be configured to articulate the articulation member 209 along multiples axes. Additional articulation actuators 228 can couple to articulation wires 220 corresponding to articulation in other directions. For example, a first articulation actuator 228 can be coupled to the articulation wire 220A configured to cause the articulation member 209 to articulate along an axis of the articulation wire 220A, a second articulators actuator 228 can be coupled to the articulation wire 220B configured to cause the articulation member 209 to articulate along an axis of the articulation wire 220B, and a third articulation actuator 228 can be coupled to the articulation wire 220C configured to cause the articulation member 209 to articulate along an axis of the articulation wire 220C. In particular, if the articulation wire 220A is disposed on a left side of the outer tubing and the articulation wire 220B is disposed on a right side of the outer tubing, then the first articulation actuator 228 can be configured to cause articulation of the distal end along the left side and the second articulation actuator 228 can be configured to cause articulation of the distal end along the right side.

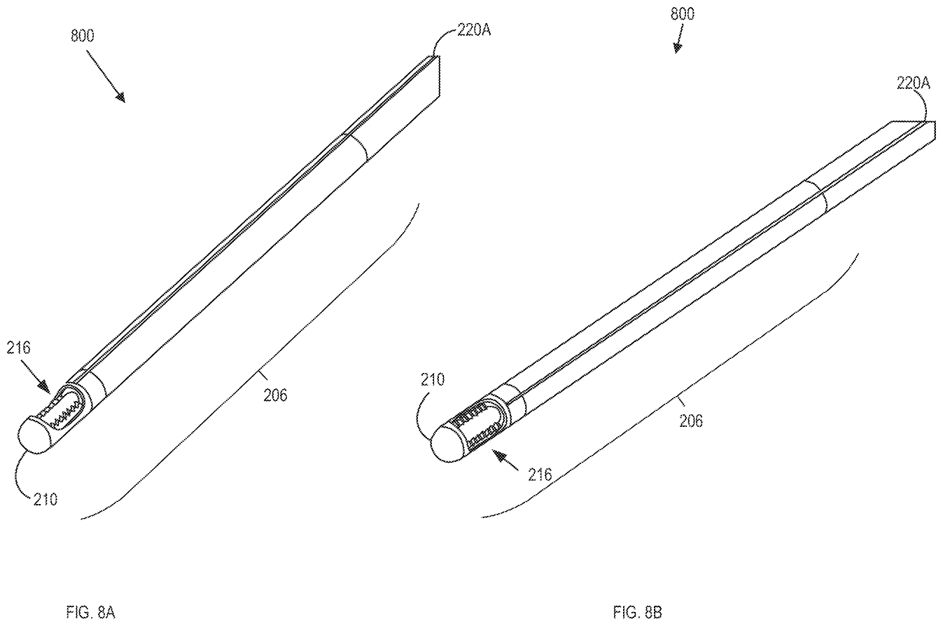

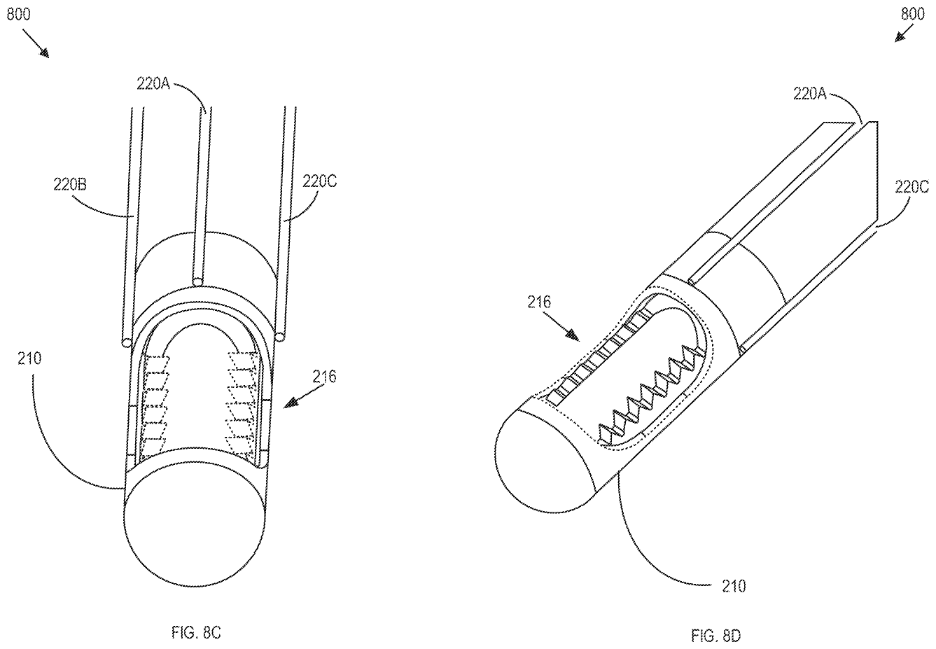

Referring to FIGS. 8A-8D, shown are perspective views of a surgical instrument 800 for maneuvering to a treatment site during a laparoscopic or hysteroscopic procedure according to embodiments of the present disclosure. The surgical instrument 800 can be similar to, and can include the same structure and functionality as the surgical instrument 400, 500, or 700, but differs in that the surgical instrument 800 dots not include securing elements 304.

Now referring generally to FIGS. 7 and 8, applying tension to one or more of the articulation wires 220 can selectively articulate the segments 302 in a direction corresponding to how the one or more articulation wires 220 are disposed. As such, the articulation wires 220 can be configured to articulate one or more of the segments 302 along multiples axes. Articulation of the segments 302 can enable the cutting assembly 210 to more effectively reach remote sample sites while reducing risk of damage to or reed functionality of components of the surgical instruments 700 and 800. The cutting assembly 210 can be manipulated while the segments 302 is articulated (e.g., changed in orientation in one or more axes) or at different times. For example, the orientation can be manipulated in a first axis perpendicular to the longitudinal axis 208 (e.g., to move the segments 302 up or down relative to a frame of reference defined with respect to longitudinal axis 208, or a second axis (e.g., to move the segments 302 left or right relative to the frame of reference defined with respect to longitudinal axis 208). The orientation can be manipulated to change an orientation of a cutting window 216 of the cutting assembly 210. Articulation can enable the cutting assembly 210 to maneuver in a greater range of positions for reaching material at the site within the subject.

Additional articulation actuators 228 can couple to articulation wires 220 corresponding to articulation in other directions. For example, a first articulation actuator 228 can be coupled to the articulation wire 220A configured to cause the segments 302 to articulate along an axis of the articulation wire 220A, a second articulation actuator 228 can be coupled to the articulation wire 220B configured to cause the segments 302 to articulate along an axis of the articulation wire 220B, and a third articulation actuator 228 can be coupled to the articulation wire 220C configured to cause the segments 302 to articulate along an axis of the articulation wire 220C. In particular, if the articulation wire 220A is disposed on a left side of the outer tubing and the articulation wire 220B is disposed on a right side of the outer tubing, then the first articulation actuator 228 can be configured to cause articulation of the distal end along the kit side and the second articulation actuator 228 can be configured to cause articulation of the distal end along the right side.

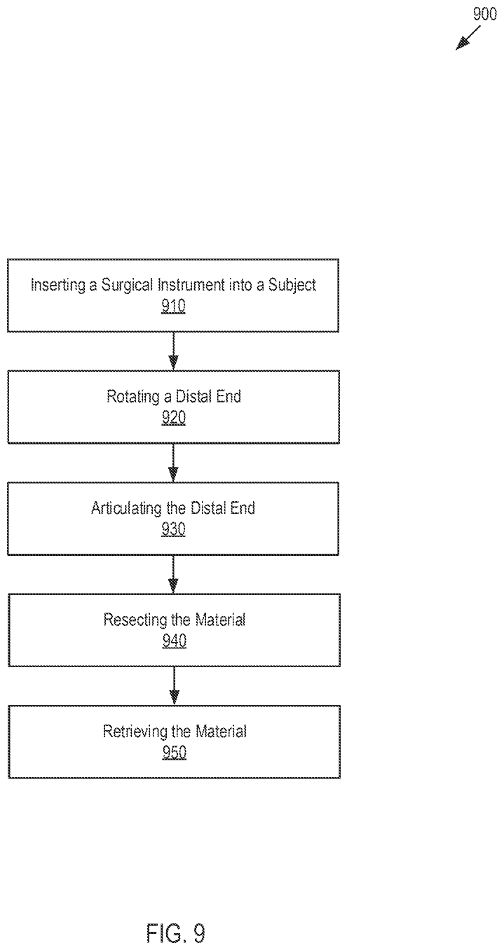

Referring to FIG. 9, a method 900 of performing a laparoscopic or hysteroscopic procedure using the surgical instrument can be shown. The method 900 can be performed using various embodiments of surgical instruments described herein. The method 900 or steps thereof can be repeated, such as to address multiple treatment sites having multiple materials to be cut, both inside and outside the vessels.

At 910, a surgical instrument (e.g. any of the surgical instruments 200-800) can be inserted into a subject. The cavity can be a body cavity or a spacing inside the body, such as the uterus, fallopian tubes, ovaries, mouth, the ear, the nose, the esophagus, etc. The cavity may be generated using at least one surgical procedure, such as a cut, a drill, or a dissection. The generated cavity can be in various different portions of the body of the subject, such as the uterus, arm, the stomach, the liver, the neck, etc.

The cavity can include a treatment site with a material to be cut from the subject. The material can include foreign material introduced into the subject, a solidified material clogging the passage of a vessel, or other material determined to be cut from the subject. The surgical instrument may inner one or more sensors, light source, or other attachments to facilitate the movement or navigation of the surgical instrument towards the treatment site. The attachments can facilitate identification of the material at the treatment site, such as to receive visual feedback to indicate the material at the treatment site. The one or more sensors can include, for example, a tilt sensor, a proximity sensor, a light sensor, a pressure sensor, a flow sensor, an impact sensor, an ultrasound sensor, a distance sensor, or other seers to facilitate an endoscopic procedure or operation. For example, a doctor or an operator may determine a location of the material using a non-intrusive imaging techniques, such as an x-ray, an ultrasound, or a computer tomography (“CT”) scan. In addition, the material can be located by navigating the surgical instrument to the treatment site and using the one or more sensors to identify the material, such as the camera or the light source. The surgical instrument may reach the treatment site or the material based on, for example, sensing a blockage within the vessel of the subject using the one or more sensor. The material may be identified using the camera of the surgical instrument and display an image on a display device external to the surgical instrument.

The procedure can include inserting a surgical instrument into the cavity of time subject. For example, a treatment site can be determined within a vessel of a subject, such as an artery, an arteriole, a capillary, a venule, or a vein. A cavity can be identified to lead to the vessel containing the treatment site. A doctor (or an operator) can insert the surgical instrument into the cavity leading to the vessel. The doctor can navigate the surgical instrument to the treatment site of the vessel. The surgical instrument can stop or terminate the navigation of the surgical instrument in response to reaching the treatment site. In some cases, the reached treatment site can be based on a camera inserted with or as a part of the surgical instrument. In some cases, the reached treatment site can be based on a length of the inserted surgical instrument. The length of the inserted surgical instrument can be determined based on a predetermined location of the treatment site via using x-ray, CT scan, ultrasound, magnetic resonance imaging (“MRI”), or other non-intrusive imaging techniques.

The surgical instrument may use the at least one sensor to navigate within the subject, determine a location of the material within the subject, and initiate the rotation of the cutting assembly in response to positioning the distal tube end of the surgical instrument at or near the treatment site. The positioning of the surgical instrument can refer to, for example, in contact with, 0.1 millimeter, 0.5 millimeter, 1 millimeter, or 1.5 millimeters from the material (or the treatment site).

The surgical instrument may be used to navigate or guide the material cutting device within the body of the subject along any torturous path. Accordingly, the surgical instrument may determine the positioning of the surgical instrument to initiate rotation for the cutting assembly to perform a cut, an extraction, or a debriding of a material within the subject. The determination to initiate the rotation can be based on, for example, a coupling or engagement between the cutting assembly and the surgical instrument.

The surgical instrument can pass the treatment site, such as through material located at the treatment site. The navigation or driving of the surgical instrument can be terminated in response to reaching, being in contact with, or passing the material or the treatment site. The laparoscopic or hysteroscopic procedure can include determining the location of a material or a treatment site. In some implementations, an operator may use at least one imaging tool to identify the location of the material, such as an x-ray, an MRI, or a CT scan. In some implementations, the operator or the doctor can utilize a camera or a scope to locate the material within the subject. The camera or the scope can be a part of the surgical instrument. For exact, the operator may insert the surgical instrument twice to perform the material naval operation or procedure. Once for identifying the material, and the pond to collect, extract, debride, or cut the material. In another example, the operator may insert the surgical instrument into the subject. The operator can navigate the surgical instrument within the subject to find the material. Once the material is found, the operator may initiate a rotation to the cutting assembly to debride and cut materials. The process of debriding the material may be referred to as removing the material. In this example, the material cutting device may be inserted once to complete the laparoscopic or hysteroscopic operation or procedure.

In some embodiments, the surgical instrument can provide or transmit at least one substance to a treatment site of a subject. The substance can include liquid, gas, or other chemical compounds. The substance may facilitate the process of degrading the material into the cut materials, for example, by exerted a gaseous substance to soften, disperse, or breakdown the material. Accordingly, the provision of the substance can assist the debriding process using the cutting assembly. In another example, the substance may assist in healing the subject, for example, by blocking a damaged portion of the vessel or by providing a medication to the treatment area within the vessel.

The outer tubing tan be an extendable and or retractable wire. The extension of the outer tubing can enable the cutting assembly to more towards a treatment site within the subject. The cutting assembly may extend or move pass the treatment site, in which an operator can terminate further extension of the outer tubing into the subject. While moving towards the treatment site, the operator may push or exert a force to the proximal end of the outer tubing. The outer tubing can be moved further inside the subject and towards the treatment site in response to the force exerted to the proximal end. The distal end of the outer tubing may be positioned a distance from the material, such as 1 millimeter, 1 inch, or 1 meter from the material.

At 920, the distal end of the surgical instrument can be rotated. The operator can grasp the handle while maneuvering the surgical instrument. A control input can be applied to the rotation actuator to cause rotation of the distal end of the outer tubing about the longitudinal axis extending through the surgical instrument. For example, the control input can rotate the proximal end of the outer tubing by −90, −80, −70, −60, −50, −40, −30, −20, −10, 10, 20, 30, 40, 50, 60, 70, 80, or 90 degrees abut the longitudinal axis 208. The operator can rotate the distal end at an angle proportional to the angle rotated by the rotation actuator. For example, the operator can rotate the outer tubing at the distal end by 10 degrees responsive to a 10 degree rotation of the rotation actuator. In another example, the operator can configure the distal end to restate according to any other configuration. For example, the operator can rotate the rotation actuator by 10 degrees to rotate the distal end by 10 degrees, and rotate the rotation actuator by 20 degrees to rotate the distal end by 13 degrees. In another example, the operator can rotate the rotation actuator by 10 degrees to rotate the distal end by 10 degrees, and rotate the rotation actuator by 25 degrees to rote the distal end by 20 degrees, in some embodiments, upon rotating the distal end, the operator can actuate a locking mechanism of the surgical instrument, the rotation actuator, or the articulation actuator to set the distal end to a target orientation.

At 930, the distal end of the surgical instrument can be articulated. A control input can be applied to the articulation actuator to cause articulation of the distal end of the outer tubing along to the articulation plane extending through the surgical instrument. The control input can cause the distal end to articulate about the articulation plane in various angles to maneuver the cutting assembly to the material. For example, the operator can articulate the distal end at −90, −80, −70, −60, −50, −40, −30, −20, −10, 10, 20, 30, 40, 50, 60, 70, 80, or 90 degrees relative to the longitudinal axis.

The operator can pull or push the articulation actuator. For example, the operator can pull or push the articulation actuator along the longitudinal axis. The operator can pull or push the articulation actuator any distance, such as 1, 2, 3, 4, 5, 6, 7, 8, 9, 10, 20, 30, 40, or 50 mm. The operator can articulate the distal end at an angle proportional to the distance moved by the articulation actuator. For example, the operator can apply a first force that causes a 30-degree articulation of the distal end, and a second force that causes a 60-degree articulation of the distal end. The second force can be stranger than the first force. For example, the first force can be 5 N, and the second force can be 10 N.

The operator can configure the control mechanisms that control the articulation of the articulation actuator. The control mechanisms can include carbide clamps, guitar mechanisms, or tensioning rods (e.g., truss rods). In some embodiments, the tensioning rod is disposed along the outer tubing. The tensioning rod can be configured to maintain a tension of the articulation wires to control the articulation member coupled to the cutting assembly. For example, the operator can configure the control mechanisms such that distal end articulates by 10 degrees responsive to a 5 mm pull of the articulation actuator. In another example, the distal end 206 is configured to articulate according to any other configuration. For example, operator can configure the distal end to articulate by 10 degrees responsive to a 5 mm pull on the articulation actuator, and articulate by 15 degrees responsive to a 10 mm pull on the articulation actuator 228. In another example, the operator can configure the distal end to articulate by 10 degrees responsive to a 5 mm pull on the articulation actuator, and articulate by 25 degrees responsive to a 10 mm pull on the articulation actuator.

In some embodiments, the operator can articulate a plurality of articulation actuators. Each of the articulation actuators can correspond to articulation in particular directions. For example, the operator can actuate a first articulation actuator to cause the distal end to articulate along a first axis of the articulation wire, a second articulation actuator to cause the distal end to articulate along a second axis, and a third articulation actuator to cause the distal end to articulate the distal end along a third axis. In some embodiments, upon articulating the distal end, the operator can actuate a locking mechanism of the surgical instrument, the rotation actuator, or the articulation actuator to set the distal end to a target orientation.

At 940, the cutting assembly can cut the material from the treatment site. The operator or a motor can rotate a flexible torque component (e.g., flexible torque component 232) disposed within the outer tubing. The flexible torque component can be coupled to the inner component. The inner component may rotate in response to receiving an exerted rotational force or torque from the articulation actuator. The flexible torque component can be configured to rotate the inner component relative to the outer component to cut the material. The removal of the material can refer to tutting, debriding, pulling, dissecting, or tearing the material from the treatment site. The cutting assembly can cut the material in response to the rotation by the inner component. The torque can be provided by the articulation actuator. The exerted rotation may traverse from the proximal end to the distal end of the surgical instrument. As an example, the articulation actuator may provide 180 degrees rotation to the surgical instrument at a proximal end, and the distal end will rotate by 180 degrees.

At 950, the material can be retrieved from the treatment site. The substances can include the cut materials, liquid, gas, or other chemical compounds within the body of the subject. The singled instrument can include actuating a vacuum source coupled to the surgical instrument to provide suction through an aspiration channel (e.g., aspiration channel 234) defined by an inner wall of the surgical instrument to cut the material from the subject via the aspiration channel. The process of retrieving the cut materials may be concurrent to the debriding process by the cutting assembly. For example, the vacuum source can initiate a vacuum to withdraw the cut materials while the tutting assembly debrides the material. The cut materials can be stored in a container or a repository in the vacuum source and/or external to the surgical instrument.

In further example, responsive to the cutting assembly debriding a material into cut materials, the surgical instrument may pull in, withdraw, or pump out the cut materials from the subject. The cut materials can be withdrawn via an aspiration channel. The process of providing the substance or withdrawing the cut material can be performed by a vacuum source. The vacuum source can be external to the surgical instrument. The vacuum source can be initiated by a signal or a mechanical trigger. A pump device may be connected to the aspiration channel configured to withdraw the cut materials from the subject. The pump device may pull a substance from a repository and push the chemical into the subject. The pump device may withdraw the cut materials into a second repository for storage.

The surgical instrument may be cut from the subject upon or bred on completion of the laparoscopic or hysteroscopic procedures or processes. The completion of the laparoscopic or hysteroscopic procedures can entail the debriding of the material, such as through an entire treatment site, or the collection of the debriding materials. For example, the treatment site can include a length of 3 inches. The surgical instrument may initiate a rotation of the cutting assembly and travel through the 3 inches of the treatment site to debride the material. While debriding the material, the surgical instrument may retrieve or draw in the cut materials into the aspiration channel. For instance, once the surgical instrument cut the material through the 0.3 inch length of the treatment site and retrieve the cut materials, the laparoscopic or hysteroscopic procedures may be completed.

### C. Systems and Methods of Telescopic Surgical Instruments.

A surgical instrument and methods thereof in accordance with the present disclosure can include components such as telescopic tubing, a cutting assembly, an actuator, a flexible torque component, and an aspiration channel. Generally, the surgical instrument may be used to provide treatment in narrow portions of a body, such as a uterus, fallopian tubes ovaries, or in some cases, to provide non-surgical treatment to a subject. The surgical instrument may be guided to a treatment she to perform a laparoscopic or hysteroscopic procedure. For example, the operator may insert the surgical instrument into a cavity of the subject, expand or rely telescopic tubes, and articulate the cutting assembly to the material.

After the surgical instrument is at the treatment site, the operator can steer the cutting assembly to the material. The location of the material can reefer to a treatment she, portion, or area for extraction, insertion, or performing other procedures using the surgical instrument. The cutting assembly can be configured to cut the material, and include an outer component and an inner component disposed within the outer component. The surgical instrument can include an articulation actuator configured to actuate the articulation wires to articulate the tubing along a longitudinal axis extending through the surgical instrument. The instrument can include a flexible torque component configured to rotate the inner component relative to the outer component to cut the materiel. The surgical instrument can include a rotation actuator configured to articulate the flexible torque component to cause the cutting assembly to cut the material. The surgical instrument can include an aspiration channel connected to a vacuum source configured to suction the material cut by the cutting assembly.

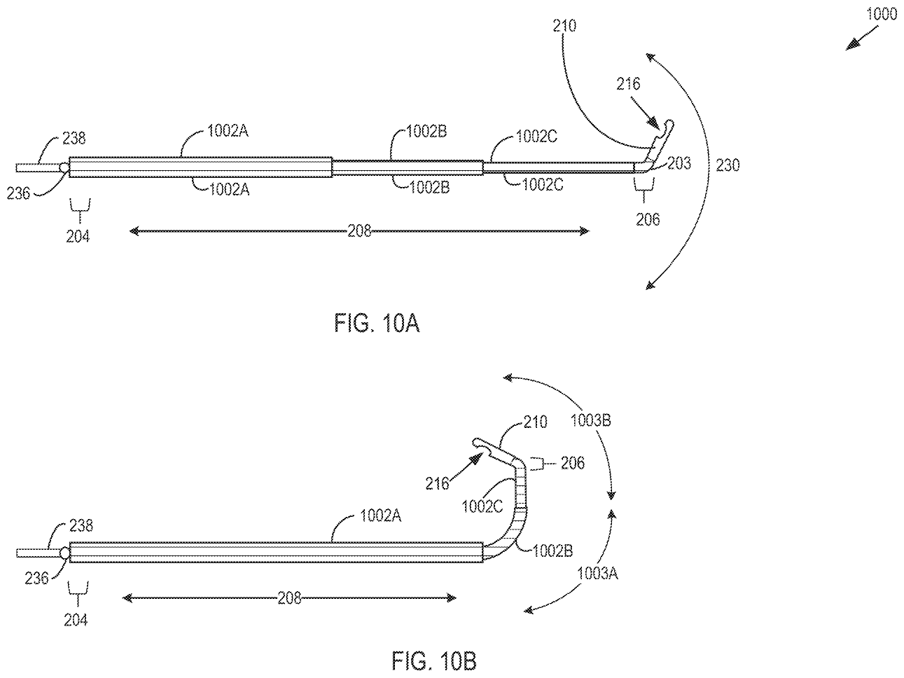

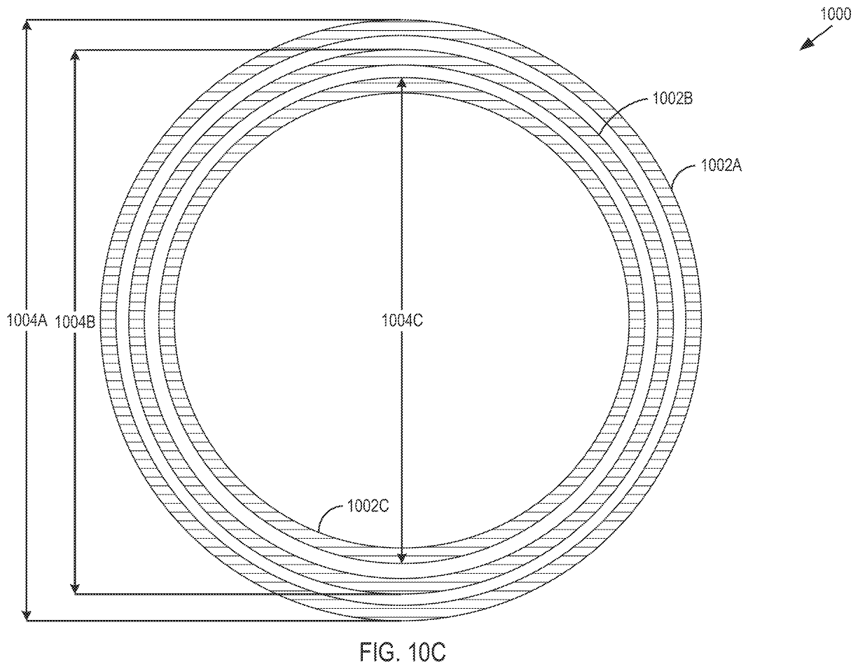

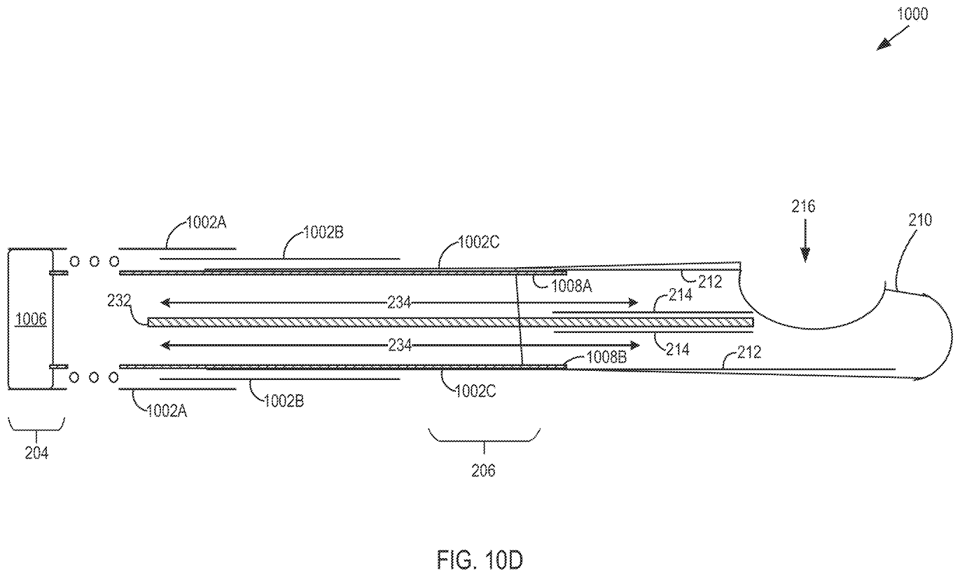

Referring to FIGS. 10A-10D, the surgical instrument 1000 can be similar to and can include the same structure and functionality as the surgical instruments 200-800, but differs in that the surgical instrument 1000 includes telescopic tubing 1002A-1002C (generally referred to as telescopic tubing 1002. The telescopic tubing 1002 on be advantageous by enabling the surgical instrument 1000 to expand and retract to reach the treatment site. The telescopic tubing 1002 includes differently sized tubes such that smaller sized telescopic tubing 1002 can retract or extend from larger telescopic tubing 1002, which enables the surgical instrument 1000 to independently articulate its distal end 206. Some of the telescopic tubing 1002 is pre-bent, which enables the operator to position the distal end 206 to the treatment site without having to articulate the telescopic tubing 1002. Moreover, the diameter of the telescopic tubing 1002 decreases closer to the distal end 206, which enables the surgical instrument 500 to reach into narrow areas to access the treatment site.

The telescopic tubing 1002 can include the same structure and functionality as the outer tubing 202, but differs in that the telescopic tubing 1002 is configured to enable each smaller sized piece of telescopic tubing 1002 to fit snugly inside the larger telescopic tubing 1002. For example, the telescopic tubing 1002 can be nitinol tubes. In another example, the telescopic tubing 1002A can enclose the telescopic tubing 1002B, which itself encloses the telescopic tubing 1002C.

Referring now to FIG. 10B, the telescopic tubing 1002 can be configured to bend or curve. For example, the telescopic tubing 1002 can be configured to bend or curve (e.g. into a straight line as shown in FIG. 10A) to enable the telescopic fits among the telescopic tubing 1002. In another example, the telescopic tubing 1002 can be configured to bend or curve upon release from the telescopic tubing 1002. For example, the telescopic tubing 1002C can be configured to bend or curve upon extending from the telescopic tubing 1002B, which itself can be configured to bend or curve upon release from the telescopic tubing 1002A. In another example and as shown in FIG. 10B, the telescopic tubing 1002A extends along the longitudinal axis 208, the telescopic tubing 1102B extends along a curve 1003A relative to the longitudinal axis 208, and telescopic tubing 1002C extends along a curve 1003B relative to the longitudinal axis 208.

Referring generally to FIGS. 10A-10D, the telescopic tubing 1002A can include the proximal end 204 and the distal end 206. The proximal end can be coupled to the external tubing 238 via the aspiration port 236. In some embodiments, a telescopic tubing 1002A has a diameter. The diameter can be less than 4 mm. The telescopic tubing 1002A can partially enclose a telescopic tubing 1002D. The telescopic tubing 1002A can enclose the telescopic tubing 1002B.

The telescopic tubing 1002B can extend out of the telescopic tubing 1002A. The telescopic tubing 1002B can be configured to extend until the distal end of the telescopic tubing 1002A encloses a proximal end of the telescopic tubing 1002B. In some embodiments, the distal end of the telescopic tubing 1002A and the proximal end of the telescopic tubing 1002B includes a hook, anchor, or other restraining mechanism to prevent the telescopic tubing 1002B from sliding out of the telescopic tubing 1002A. The telescopic tubing 1002B can be configured to retract into the telescopic tubing 1002A until the proximal end of the telescopic tubing 1002A encloses the proximal end of the telescopic tubing 1002B. In some embodiments, a telescopic tubing 1002B has a diameter. The diameter can be less than the diameter of the telescopic tubing 1002A. The telescopic tubing 1002B can enclose the telescopic tubing 1002C.

The telescopic tubing 1002C can extend out of the telescopic tubing 1002B. The telescopic tubing 1002C can be configured to extend until the distal end of the telescopic tubing 1002C encloses a proximal end of the telescopic tubing 1002C. In some embodiments, the distal end of the telescopic tubing 1002B and the proximal end of the telescopic tubing 1002C includes a hook, anchor, or other restraining mechanism to prevent the telescopic tubing 1002C from sliding out of the telescopic tubing 1002B. The telescopic tubing 1002C can be configured to retract into the telescopic tubing 1002B until the proximal end of the telescopic tubing 1002B encloses the proximal end of the telescopic tubing 1002C. In some embodiments, a telescopic tubing 1002C has a diameter. The diameter can be less than the diameter of the telescopic tubing 1002B. The telescopic tubing 1002B can enclose the telescopic tubing 1002C.

The telescopic tubing 1002 can be coupled to the cutting assembly 210. The cutting assembly 210 can be configured to retrieve material fin a treatment site. The material can be suctioned via the telescopic tubing to the aspiration port 236 and to the external tubing 238.

Referring to FIG. 10C, shown a cross sectional tunnel view of the telescopic tubing 1002A-1002C with respective diameters 1004A-1004C. The diameter 1004A of telescopic tubing 1002A can be less than 4 mm. The drier 1004B of telescopic tubing 1002B can be less than the diameter 1004A of telescopic tubing 1002A. The diameter 1004C of telescopic tubing 1002C can be less than the diameter 1004B of telescopic tubing 1002B.

Now referring generally to FIGS. 10A-10D, a telescopic actuator 1006 can be configured to control the expansion or retraction of the telescopic tubing 1002. The telescopic actuator 1006 can include the same structure and functionality as the actuator 226 or 228 (and can be coupled to or include the handle 224). The telescopic actuator 1006 can be coupled to telescopic wires 1008A and 1008B (generally referred to as telescopic wires 1008). The actuator can push or pull the telescopic wires 1008 to expand or retract the telescopic tubing 1002. The telescopic actuator 1006 can include more than one actuator such that each actuator is coupled to a respective telescopic wire 1008.

The telescopic wires 1008 can include, the same structure and functionality as the articulation wires 220, but differ in that the telescopic wires 1008 can be configured to cause expansion or retraction of the telescopic tubing 1002. In some embodiments, the telescopic wires 1008 can be configured with telescopic functionality like the telescopic tubing 1002. For example, the telescopic wires 1008 can expand to expand the telescopic tubing 1002, or retract to retract the telescopic tubing 1002. The telescopic wires 1008 can be coupled to at least the most distal pan of the telescopic tubing 1002 (e.g., telescopic tubing 1002C) or the cutting assembly 210.

The telescopic actuator 1006 can be configured to actuate the telescopic wires 1008 to cause expansion or retraction of the telescopic tubing 1002. In some embodiments, the actuations can be control inputs, pushes, pulls, twists, or rotations. For example, pushing the telescopic wires 1008 can cause expansion of the telescopic tubing 1002, and pulling the telescopic wires 1008 can cause retraction of the telescopic tubing 1002. In another example, rotating the telescopic wires 1008 in a first direction (e.g., clockwise) can cause expansion of the telescopic tubing 1002, and rotating the telescopic wires 1008 in a second direction (e.g., counterclockwise) can cater retraction of the telescopic tubing 1002.

The telescopic actuator 1006 can apply a first control input (e.g., push the telescopic wires 1008) to extend the cutting assembly 210 and the telescopic tubing 1002C coupled thereof out of the telescopic tubing 1002B. The telescopic actuator can apply a second control input (e.g., push the telescopic wires 1008) to further extend the cutting assembly 210 and the telescopic tubing 1002C coupled thereof, which would cause the telescopic tubing 1002B to extend out of the telescopic tubing 1002A. Conversely, the telescopic actuator can apply a third control input (e.g., pull the telescopic wires 1008) to retract the cutting assembly 210 and the telescopic tubing 1002C coupled thereof into the telescopic tubing 1002B. The telescopic actuator can apply a second control input (e.g. pull the telescopic wires 1008) to further retract the cutting assembly 210 and the telescopic tubing 1002C coupled thereof, which would cause the telescopic tubing 1002B to retract into the telescopic tubing 1002A.

The telescopic actuator 1006 can be configured to rotate the telescopic tubing 1002 about the longitudinal axis 208 extending through the surgical instrument 200 responsive to receiving a first control input at the telescopic actuator 1006. The telescopic actuator 1006 can be coupled to the proximal end oft the telescopic tubing 1002. The telescopic actuator 1006 can be coupled to the articulation wires 220. The telescopic actuator 1006 can be a knob, tube, handle, grip, or any other surface configured to receive control inputs from the operator. The control inputs can cause time telescopic actuator 1006 to mote the proximal end 204 of the telescopic tubing 1002 about to the longitudinal axis 203. In another example, the control inputs can cam the telescopic actuator 1006 to pull on the articulation wires 220 to rotate the telescopic tubing 1002 about to the longitudinal axis 208. For example, the control inputs can rotate the proximal end 204 of the telescopic tubing 1002 by −90, −80, −70, −60, −50, −40, −30, −20, −10, 10, 20, 30, 40, 50, 60, 70, 80, or 90 degrees about the longitudinal axis 208.

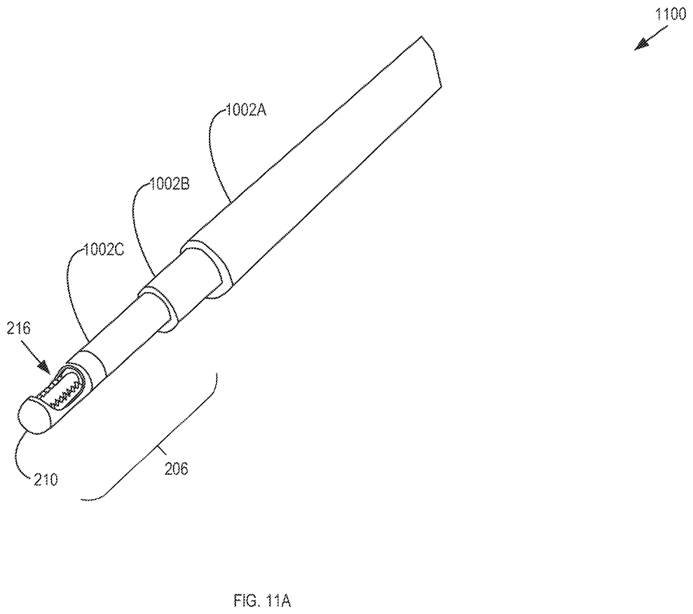

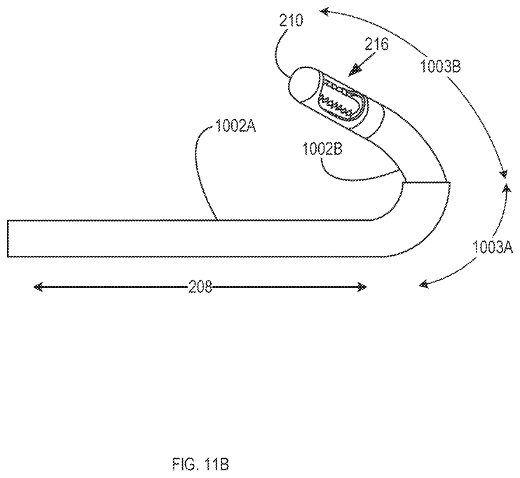

Referring to FIGS. 11A and 11B, shown are perspective views of a surgical instrument 1100 having a telescopic configuration for maneuvering to a treatment site during a laparoscopic or hysteroscopic procedure according to embodiments of the present disclosure. The surgical instrument 1100 can be similar to, and can include the same structure and functionality as the surgical instrument 200-800 and 1000.

Referring to FIG. 11A, the telescopic tubing 1002 can have a telescopic configuration such that sections of the telescopic tubing 1002 closer to the proximal end 204 enclose sections that are closer to the distal end 206. For example, the telescopic tubing 1002A encloses telescopic tubing 1002B, which encloses telescopic tubing 1002C, which is coupled to the cutting assembly 210.

Referring to FIG. 11B, shown is a perspective view of telescoping tubing 1002 of the surgical instrument 1100 that is pre-bent for maneuvering to a treatment site during a laparoscopic or hysteroscopic procedure according to embodiments of the present disclosure. For example, the telescopic tubing 1002A is pre-bent along the axis 1003A. The telescopic tubing 1002B extends out of the telescopic tubing 1002A along the axis 1003B.

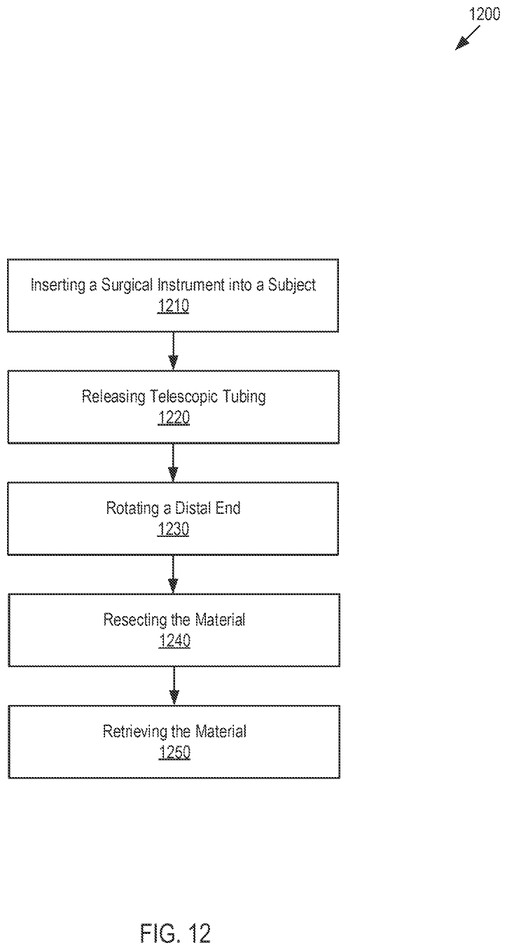

Referring to FIG. 12, a method 1200 of performing a laparascopic or hysteroscopic procedure using the surgical instrument can be shown. The method 1200 can be performed using various embodiments of surgical instruments described herein. The method 1200 or steps thereof can be repeated, such as to address multiple treatment sites having multiple materials to be cut, both inside and outside the vessels.

At 1210, a surgical instrument (e.g., any of the surgical instruments 1000 or 1100) can be inserted into a subject. The cavity can be a body cavity or a spacing inside the body, such as the uterus, fallopian tubes, ovaries mouth, the ear, the nose, the esophagus, etc. The cavity may be generated using at least one surgical procedure, such as a cut, a drill, or a dissection. The generated cavity can be in various different portions of the body of the subject, such as the uterus, arm the stomach, the liver, the neck, etc.

The cavity can include a treatment site with a material to be cut froth the subject. The material can include foreign material introduced into the subject, a solidified material clogging the passage of a vessel, or other material determined to be cut from the subject. The surgical instrument may include one or more sensors, light source, or other attachments to facilitate the movement or navigation of the surgical instrument towards the treatment site. The attachments can facilitate identification of the material at the treatment site, such as to receive visual feedback to indicate the material at the treatment site. The one or more sensors can include, for example, a tilt sensor, a proximity sensor, a light sensor, a pressure sensor, a flow sensor, an impact sensor, an ultrasound sensor, a distance sensor, or other sensors to facilitate an endoscopic procedure or operation. For example, a doctor or an operator may determine a location of the material using a non-intrusive imaging techniques, such as an x-ray, an ultrasound, or a computer tomography (“CT”) scan. In addition, the material can be located by navigating the surgical instrument to the treatment site and using the one or more sensors to identify the material, such as the camera or the light source. The surgical instrument may reach the treatment site or the material based on, for example, sensing a blockage within the vessel of the subject using the one or more sensor. The material may be identified using the camera of the surgical instrument and display an image on a display device external to the surgical instilment.

The procedure can include inserting a surgical instrument into the cavity of the subject. For example, a treatment site can be determined within a vessel of a subject, such as an artery, an arteriole, a capillary, a venule, or a vein. A cavity can be identified to lead to the vessel containing the treatment site. A doctor (or an operator) can insert the surgical instrument into the cavity leading to the vessel. The doctor can navigate the surgical instrument to the treatment site of the vessel. The surgical instrument can stop or terminate the navigation of the surgical instrument in response to reaching the treatment site. In some cases, the reached treatment site can be based on a camera inserted with or as a part of the surgical instrument. In some cases, the reached treatment site can be based on a length of the inserted surgical instrument. The length of the inserted surgical instrument can be determined based on a predetermined location of the treatment site via using x-ray, CT scan, ultrasound, magnetic resonance imaging (“MRI”), or other non-intrusive imaging techniques.

The surgical instrument may use the at least one sensor to navigate within the subject, determine a location of the material within the subject, and initiate the rotation of the cutting assembly in response to positioning the distal tube end of the magical instrument at or near the treatment site. The positioning of the surgical instrument can refer to, for example, in contact with, 0.1 millimeter, 0.5 millimeter, 1 millimeter, or 1.5 millimeters from the material (or the treatment site).

The surgical instrument may be used to navigate or guide the material cutting device within the body of the subject along any torn path. Accordingly, the surgical instrument may determine the positioning of the surgical instrument to initiate a rotation for the cutting assembly to perform a cut, an extraction, or a debriding of a material within the subject. The determination to initiate the rotation can be based on, for example, a coupling or engagement between the cutting assembly and the surgical instrument.

The surgical instrument can pass the treatment site, such as through material located at the treatment site. The navigation or driving of the surgical instrument can be terminated in response to reaching, being in contact with, or passing the material or the treatment site. The laparoscopic or hysteroscopic procedure can include determining the location of a material or a treatment site. In some implementations, an operator may use at least one imaging tool to identify the location of the material, such as an x-ray, an MRI, or a CT scan. In some implementations, the operator or the doctor can utilize a camera or a scope to locate the material within the subject. The camera or the scope can be a part of the surgical instrument. For exact, the operator may insert the surgical instrument twice to perform the material naval operation or procedure. Once for identifying the material, and the pond to collect, extract, debride, or cut the material. In another example, the operator may insert the surgical instrument into the subject. The operator can navies the surgical instrument within the subject to find the material. Once the material is found, the operator may initiate a rotation to the cutting assembly to debride and cut materials. The process of debriding the material may be referred to as removing the material. In this example, the material cutting device may be inserted once to complete the laparoscopic or hysteroscopic operation or procedure.

In some embodiments, the surgical instrument can provide or transmit at least one substance to a treatment site of a subject. The substance can include liquid, gas, or other chemical compounds. The substance may facilitate the process of debriding the material into the cut materials, for example, by exerted a gaseous substance to soften, disperse, or breakdown the material. Accordingly, the provision of the substance can assist the debriding process using the cutting assembly. In another example, the substance may assist in healing the subject, for example, by blocking a damaged portion of the vessel or by providing a medication to the treatment area within the vessel.

The telescopic tubing can be an extendable and or retractable wire. The extension of the telescopic tubing can enable the cutting assembly to move towards a treatment site within the subject. The cutting assembly may extend or move pass the treatment site, in which an operator can terminate further extension of the telescopic tubing into the subject. While moving towards the treatment site, the operator may push or exert a force to the proximal end of the telescopic tubing. The telescopic tubing can be moved further inside the subject and towards the treatment site in response to the force exerted to the proximal end. The distal end of the telescopic tubing may be positioned a distance from the material, such as 1 millimeter, 1 inch, or 1 meter from the material.

At 1220, the telescopic tubing of the surgical instrument can be released. The operator can apply control inputs to the telescopic actuator to actuate the telescopic wires to cause expansion or retraction of the telescopic tubing. In some embodiments, the actuations can be control inputs, pushes, pulls, twists, or rotations. For example, the operator tan push the actuator to push the telescopic wires to cause expansion of the telescopic tubing. In another example, the operator can pull the telescopic wires to cause retraction of the telescopic tubing. In another example, the operator van rotate the telescopic wires in a first direction (e.g., clockwise) to cause expansion of the telescopic tubing, and rotating the telescopic wires in a second direction (e.g., counterclockwise) can cause retraction of the telescopic tubing.

The telescopic actuator can apply a first control input (e.g., push the control wires) to extend the cutting assembly and the small telescopic tubing coupled thereof out of the medium telescopic tubing. The telescopic actuator can apply a second control input (e.g., push the control wires) to further extend the crating assembly and the small telescopic tubing coupled thereof, which would cause the medium telescopic tubing to extend out of the large telescopic tubing.

At 1230, the distal end of the surgical instrument can be rotated. The operator can grasp the handle while maneuvering the surgical instrument. A control input can be applied to the telescopic actuator to cause rotation of the distal end of the telescopic tubing about the longitudinal axis extending through the surgical instrument. The telescopic actuator can be configured to rotate the telescopic tubing about the longitudinal axis extending through the surgical instrument responsive to receiving a first control input at the telescopic actuator. The telescopic actuator can be coupled to the proximal end of the telescopic tubing. The telescopic actuator can be coupled to the articulation wires. The telescopic actuator can be a knob, tube, handle, grip, or any other surface configured to receive control inputs from the operator. The control inputs can cause the telescopic actuator to rotate the proximal end of the telescopic tubing about to the longitudinal axis. In another example, the control inputs can cause the telescopic actuator to pull on the articulation wires to rotate the telescopic tubing about to the longitudinal axis. For example, the control inputs can rotate the proximal end of the telescopic tubing by −90, −80, −70, −60, −50, −40, −30, −20, −10, 10, 20, 30, 40, 50, 60, 70, 80, or 90 degrees about the longitudinal axis 208.

The operator can rotate the distal end at an angle proportional to the angle rotated by the telescopic actuator. For example, the operator can rotate the telescopic tubing at the distal end by 10 degrees responsive to a 10 degree rotation of the telescopic actuator. In another example, the operator can configure the distal end to rotate according to any other configuration. For example, the operator can rotate the telescopic actuator by 10 degrees to rotate the distal end by 10 degrees, and rotate the telescopic actuator by 20 degrees to rotate the distal end by 15 degrees. In another example, the operator can rotate the telescopic actuator by 10 degrees to rotate the distal end by 10 degrees, and rotate the telescopic actuator by 25 degrees to rotate the distal end by 20 degrees.

At 1240, the cutting assembly can cut the material from the treatment site. The operator or a motor can rotate a flexible torque component (e.g., flexible torque component 232) disposed within the telescopic tubing. The flexible torque component can be coupled to the inner comment. The inner component may rotate in response to receiving an exerted rotational force or torque from the articulation actuator. The flexible torque component can be configured to rotate the inner component relative to the outer component to cut the material. The removal of the material can refer to cutting, debriding, pulling, dissecting, or tearing the material from the treatment site. The cutting assembly can cut the material in response to the rotation by the inner component. The torque can be provided by the articulation actuator. The exerted rotation may traverse from the proximal end to the distal end of the surgical instrument. As an example, the articulation actuator may provide 180 degrees rotation to the surgical instrument at a proximal end, and the distal end will rotate by 180 degrees.

At 1250, the material can be retrieved from the treatment site. The substances can include the cut materials, liquid, gas, or other chemical compounds within the body of the subject. A vacuum some coupled to the surgical instrument can provide suction through an aspiration channel (e.g., aspiration channel 234) defined by an inner wall of the surgical instrument to cut the material from the subject via the aspiration channel. The process of retrieving the cut materials may be concurrent to the debriding process by the cutting assembly. For example, the vacuum source can initiate a vacuum to withdraw the cut materials while the cutting assembly debrides the material. The cut materials can be stored in a container or a repository in the vacuum source and/or external to the surgical instrument.

In further example, responsive to the cutting assembly debriding a material into cut materials, the surgical instrument may pull in, withdraw, or pump out the cut materials from the subject. The cut materials can be withdrawn via an aspiration channel. The process of providing the substance or withdrawing the cut material can be performed by a vacuum source. The vacuum source can be external to the musical instrument. The vacuum source can be initiated by a signal or a mechanical trigger. A pump device may be connected to the aspiration channel configured to withdraw of the cut materials from the subject. The pump device may pull a substance from a repository and push the chemical into the subject. The pump device may withdraw the cut materials into a second repository for storage.

The surgical instrument may be cut horn the subject upon or based on completion of the laparascopic or hysteroscopic procedures or processes. The telescopic actuator can apply, a third control input (e.g., pull the control wires) to retract the cutting assembly and the small telescopic tubing coupled thereof into the medium telescopic tubing. The telescopic actuator can apply a second control input (e.g., pull the control wires) to further retract the cutting assembly and the small telescopic tubing coupled thereof, which would cause the medium telescopic tubing to retract into the large telescopic tubing.

The completion of the laparoscopic or hysteroscopic procedures can entail the debriding of the material, such as through an entire treatment site, or the collection of the debriding materials. For example, the treatment site can include a length of 3 inches. The surgical instrument may initiate a rotation of the cutting assembly and travel through the 3 inches of the treatment site to debride the material. While debriding the material, the surgical instrument may retrieve or draw in the cut materials into the aspiration channel. For instance, once the surgical instrument cut the material through the 3 inch length of the treatment site and retrieve the an materials, the laparoscopic, hysteroscopic procedures may be completed.

### D. Systems and Method for an Integrated Steerable Instrument for Maneuvering to a Treatment Site

It is difficult to maneuver a cutting assembly at a distal end of a surgical instrument to a desired material at a treatment site while retaining the ability of the cutting assembly at the distal end of the surgical instrument to be properly operated, and even more difficult to use surgical instruments with tither surgical touts in cavities and other narrow or tortuous treatment sites. A steerable instrument and methods thereof in accordance with the present disclosure can address this problem by enabling independent articulation of a distal end of the steerable instrument while retaining the ability of a cutting assembly at the distal end of the steerable instrument to be properly operated. The flexibility and small diameter of the steerable instrument enables the steerable instrument to navigate through a cavity or working channel of the surgical tool. For surgical tools that cannot receive the steerable instrument through their working channel, the attachment members enable the steerable instrument to navigate or along the external side of the surgical tool. While the steerable instrument is disposed in the cavity, the working channel, or the attachment members along the flexible tool, the steerable instrument can then articulate the distal end and actuate the cutting assembly thereof without articulating the rest of the steerable instrument to avoid damage or kinks to the cavity, surgical tool, or the steerable instrument itself.

The steerable instrument can include components such as a cutting assembly, a flexible outer tubing, a first connector, a flexible torque component, a second connector, and an aspiration channel. Generally, the steerable instrument may be used to provide treatment in narrow portions of a body, such as a uterus, fallopian tubes, ovaries, or in some cases, to provide norm-surgical treatment to a subject. The steerable instrument may be guided to a treatment site to perform a laparoscopic or hysteroscopic procedure. For example, the operator may insert the steerable instrument into a cavity of the subject and articulate the cutting assembly to a treatment site. In some embodiments, the operator inserts the steerable instrument into a channel of a surgical total. In other embodiments, the steerable instrument includes at least one attachment giber configured to attach the steerable instilment along the surgical tool.

After the steerable instrument is at the treatment site, the operator can steer the cutting assembly, to the material. The location of the material can refer to a treatment site, portion, of area for extraction, inspection, or performing other procedure using the steerable instrument 1300. The cutting assembly can be configured to cut the material and includes an outer sheath and an inner sheath disposed within the outer sheath. The steerable instrument can include a first connector configured to articulate the flexible outer tubing relative to a longitudinal axis extending through the steerable instrument. The steerable instrument can include a flexible torque component configured to rotate the inner sheath relative to the outer sheath to cut the material. The steerable instrument can include a second connector configured to rotate the flexible torque component to cause the cutting assembly to cut the material. The steerable instrument can include an aspiration channel connected to a vacuum source configured to suction the material cut by the cutting assembly.

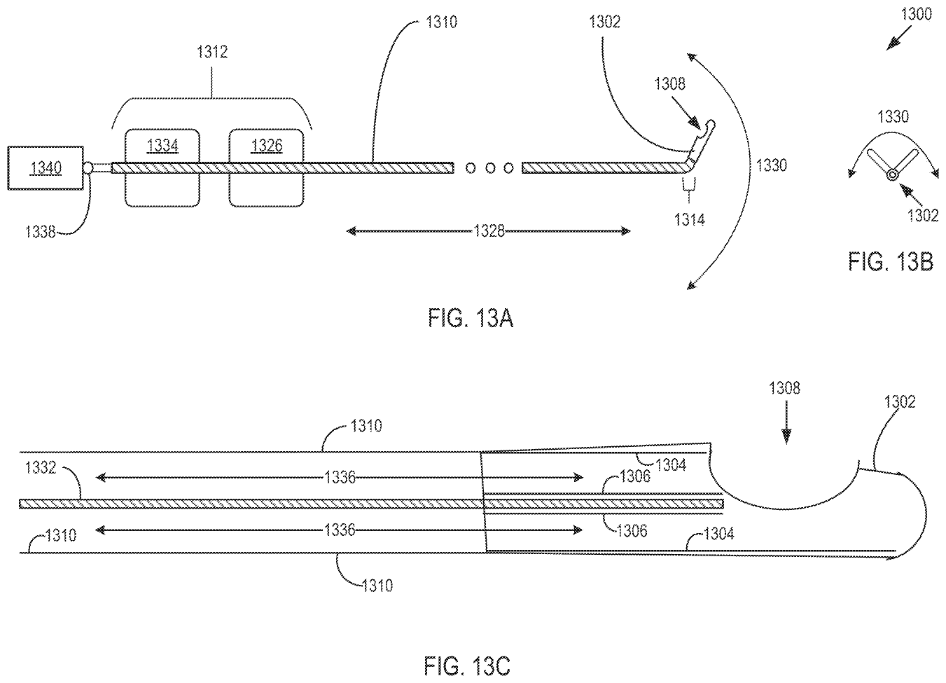

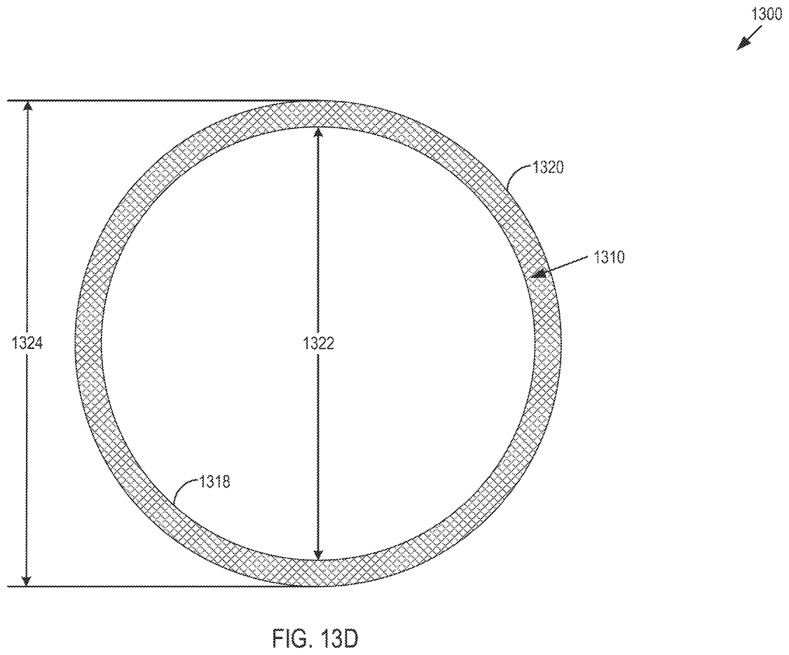

Referring to FIGS. 13A-13D, shown are views of the steerable instrument 1300 for maneuvering to a treatment site during a laparoscopic or hysteroscopic procedure according to embodiments of the present disclosure. The steerable instrument 1300 can include a cutting assembly 1302, which can include an outer sheath 1304 and an inner sheath 1306. The outer sheath 1304 can define a cutting window 1308. The steerable instrument 1300 can include a steerable tubing 1310. The steerable tubing 1310 can include a proximal end 1312, a distal end 1314, a base layer 1318, a top layer 1320, an inner diameter 1322, and an outer diameter 1324. The steerable instrument 1300 can include a first connector 1326, a longitudinal axis 1328, an articulation axis 1330, a flexible torque component 1332, a second connector 1334, an aspiration channel 1336, and an aspiration port 1338 configured to couple to a vacuum source 1340.

For example, referring leer to FIG. 13A, for performing a procedure to cut material from the treatment site, the steerable tubing 1310 can be introduced into a cavity of the subject. The cutting assembly 1302 can be introduced to the treatment site. The operator can use the first connector 1326 to maneuver the cutting assembly 1302 along the articulation axis 1330 to the material. The operator can use the second connector 1334 to actuate the cutting assembly 1302 to cut the material. A motor can also initiate a rotation to rotate the cutting assembly 1302. The cutting assembly 1302 can rotate in response to the initiated rotation by the second connector 1334 or the motor. The material may be extracted, cut, collected, or investigated by the steerable instrument 1300. In some caste the cutting assembly 1302 can extract, pull, or collect the material into the cutting window 1308. The vacuum source 1340 can suction the material into the aspiration channel 1336 extending from the cutting window 1308 to the aspiration port 1338.

Referring to FIG. 13A in conjunction with FIG. 13B, the cutting assembly 1302 can be configured to cut material from a subject. The cutting assembly 1302 can be coupled to or located at the distal end of the steerable instrument 1300. The cutting assembly 1302 can be a distance from the distal end 1314 of the flexible outer tubing. For example, the distance can be 1, 2, 3, 4, 5, 6, 7, 8, 9, or 10 millimeters. The cutting assembly 1302 can include the blade (or a fan blade). The cutting assembly 1302 can include one or more blades, such as two blades as shown in FIG. 2B. The cutting assembly 1302 can include a fan, an axial cutter, a drill, a hook, a scoop, a reamer, a miller cutter, or other cutting tools or devices. The cutting assembly 1302 can be referred to as a debriding component, a cutter, a removal tool, or an extractor. The cutting assembly 1302 can include a blade. The blade can be composed of one or mote materials for cutting or dissecting a material, such as a steels, plastics, carton fibers, titanium, aluminums, metals, or other alloys for performing laparoscopy or hysteroscopy operations. The cutting assembly 1302 can perform actions, including but not limited to, cutting, snaring, shredding, slicing, shattering, either entirely or partially, are also examples of debriding. Accordingly, the cutting assembly 1302 may be a component that is capable of cutting, snaring, shredding, slicing, or shattering from a surface of the body of the subject. As such, the cutting assembly 1302 may be implemented as a forceps, scissor, knife, snare, shredder, or any other component that can debride.

The cutting assembly 1302 may be actuated such that the cutting assembly 1302 may be operated through the translation of mechanical forces exerted by an operator or automatically actuated, using a turbine, a motor (e.g., electrical motor), or any other force generating component to actuate the debriding component. The cutting assembly 1302 can be configured to cut at various speeds, such as 5000 rotations per minute (“RPM”), 10,000 RPM, 130,000 RPM, or 50,000 RPM. The cutting assembly 1302 may be manually operated or may utilize any other means of debriding material such that the cut material are capable of being retrieved from the treatment site via the steerable tubing 1310. The cutting assembly 1302 can cut the material into small enough pieces, which may be retrieved via the steerable instrument 1300 such that the amble instrument 1300 does not need to be cut from the subject to collect the cut material. It should be appreciated that the cutting assembly 1302 is able to rotate a specific degree and with a specific torque, equivalent or matching the rotation and torque of the motor or operator. Accordingly, the cutting assembly 1302 can provide cutting precision, control, and power consumption. For example, the cutting assembly 1302, coupled to the cutting assembly 1302, can rotate a number of degrees with a specific torque equivalent to an operator providing the degrees and torque to the motor. For example, the operator or motor may initiate a 30-degrees oration. The rotation, force, and torque can be exerted from the motor to the cutting assembly 1302. The cutting assembly 1302 can receive the exerted rotation. Accordingly, the cutting assembly 1302 may rotate 30-degrees based on the exerted rotation, force, and torque of the motor of operator.

The cutting assembly 1302 can include at least one sensor, such as a proximity sensor, a light sensor, a pressure sensor, a radar sensor, a flow sensor, a flex sensor, an impact sensor, a distance sensor, or other sensor configured to inspect, examine, sense, or navigate through a body of a subject. The cutting assembly 1302 may include a light source and a recording device or capturing device (e.g., a camera or a scope) to collect visual information from an invective of the body of the subject. The light source can include a light emitting diode (“LED”), incandescent lamps, compact fluorescent, halogen, neon, or other types of lighting elements. The steerable instrument 1300 or the cutting assembly may emit light and initiate recording using the light source and the recording device. The cutting assembly 1302 may receive at least one visual information from the camera and transmit the at least one visual information to the display device. The display device can generate or display the images based on the received visual information for an operator or a doctor to view inside the body of the subject during an operation. In some embodiment, the cutting assembly 1302 can be equipped with an injectable dye component through which the operator can use to determine the extent of narrowing under fluoroscopic guidance or to mark a particular region within the subject. In other embodiments, the operator can mark a particular region with the cutting assembly 1302, without the use of an injectable dye.

Referring to FIG. 13A in conjunction with FIG. 13C, the cutting assembly 1302 can include the outer sheath 1304 and the inner sheath 1306 disposed within the outer sheath 1304. The outer sheath 1304 can be configured to pass fluid. The outer sheath 1304 can be a component, cover, an outer tube, a shell, or a main body of the cutting assembly 1302. The outer sheath 1304 cars be shaped or formed to, for example, a cylinder, a prism, a cone, or other shapes. The outer sheath 1304 can be flexible. The outer sheath 1304 can bend and flex to any degree. In some embodiments, the outer sheath 1304 can bend and flex to 10, 20, 30, 40, 50, 60, 70, 80, 90, 100, 110, 120, 130, 140, 150, 160, 170, or 180 degrees. The outer math 1304 can include a thickness. The thickness can be 10 nanometers, 20 nanometers, 1 millimeter, 2 millimeter, 3 millimeter, 4 millimeters, or 5 millimeters. The outer sheath 1304 can include a width. The width can be 1 millimeter, 2 millimeters, 3 millimeter, 4 millimeters, 5 millimeters, or 1 centimeter. The outer sheath 1304 can include a length. The length can be 1 meters, 2 meters, 3 meters, 4 meters, 5 meters, 6 meters 7 meters, 8 meters, 9 meters, 10 meters, 50 meters, 100 meters, etc. The outer sheath 1304 can include a cross-sectional area, such as 0.6 millimeters squared, 1 millimeters squared, 1.9 millimeters fired, etc. The outer sheath 1304 can be composed of materials, such as metal, steel, plastic, rubber, glass, carbon fiber, titanium, aluminum, or other alloys.

The outer sheath 1304 can at least partially surround the inner sheath 1306. In some embodiments, the inner sheath 1306 cuts any material suctioned into or otherwise entering the outer sheath 1304. The outer sheath 1304 can be a component, cover, a tube, or a shell. The inner sheath 1306 can include an opening such that material cut by the cutting assembly 1302 enters via the opening. The inner sheath 1306 can include a length similar to or less than the cuter sheath 1304. The length can be 1, 2, 3, 4, 3, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 cm. The inner sheath 1306 can be designed to facilitate debriding one or more materials and removing the cut materials in a single operation. The inner sheath 1306 can be disposed within the outer sheath 1304. The inner sheath 1306 can couple with the outer sheath 1304. The inner sheath 1306 can be composed of a similar material as the outer sheath 1304. The inner sheath 1306 can be flexible, similar to the outer sheath 1304.

The outer sheath 1304 can define the cutting window 1308. The outer sheath 1304 can include the cutting window 1308, at a distal end of the cutting assembly 1302. A portion of the radial wall of the outer sheath 1304 can define the cutting window 1308 that extends around a portion of the radius of the outer sheath 1394. In some embodiments, the operator can receive or retie cut materials through the cutting window 1308.

The wing window 1308 can be configured to enable the cutting assembly 1302 to cut, dissect, or debride the material. For example, the cutting assembly 1302 can initiate the debriding or cutting process by rotating the cutting through the material to receive the material in the cutting window 1308. The cutting window 1308 can be positioned at a side of the cutting assembly 1302. The cutting window 1308 can be configured to enable tangential or side cutting of material with respect to the movement of the cutting assembly 1302. In some embodiments, the outer sheath 1304 can include the cutting window 1308. The cutting window 1308 can include a hollow structure with a shape, such as a circle, an oval, a rectangle, or other geometric shape for exposing the blades of the cutting assembly 210. The cutting window 1308 can include a diameter. The diameter can be 1 millimeter, 2 millimeters, 3 millimeters, 4 millimeters, or 5 millimeters. The cutting window 1308 can include a cut out, which can be a portion of the cutting assembly 1302. For example, the tutting window 1308 can include a 0.4 millimeters cut out.

The steerable tubing 1310 can be a navigation wire, a motorized wire, or a braid. The steerable tubing 1310 can include nitinol or other memory material such that articulation of the proximal end 1312 would cause articulation of the distal end 1314. The steerable tubing 1310 can include rubber, cloth, metal, steel, plastic, titanium, nickel, or carbon fiber. The steerable tubing 1310 can be a braided sheath. In some embodiments, the steerable tubing 1310 can also include a lining that fits around the steerable tubing 1310. In some embodiments, the lining can prevent air or other fluids to seep between the steerable tubing 1310. The steerable tubing 1310 can be coupled to the outer sheath 1304. In some implementations, the steerable instrument 1300 can be surrounded by a sheath or lining to avoid frictional contact between the outer surface of the flexible torque component 1332 and other surfaces. In some implementations, the steerable instrument 1300 can be coated with Polytetrafluorethylene (“PFTE”) to reduce fictional contact between the outer surface of the steerable instrument 1300 and other surfaces such as the inner wall of the subject.

The stable tubing 1310 can be maneuvered within the subject. The insertion of the steerable tubing 1310 can be through the opening or the cavity. The steerable tubing 1310 can be turned, bent, or otherwise navigated through curvatures of the subject. For example, the steerable tubing 1310 can be maneuvered into a curved portion of the subject. The steerable tubing 1310 can be in contact with the subject, such that the steerable tubing 1310 can navigate through the curved portion of the subject. The steerable tubing 1310 can be bent or turned in response to reaching or being in contact with the cued portion, such that the steerable tubing 1310 curves through the curved portion while navigating. For example, the bodily cavity can include curves, bumps, or otherwise non-linear paths to a treatment site. The treatment site can be located past the non-linear path within the subject. The steerable tubing 1310 can push, bump, or impact within the bodily cavity to turn through the non-linear path of the cavity. In some cases, the steerable tubing 1310 can be navigated through a cavity by bouncing, turning, or adjusting a navigation direction in response to at least a contact with the cavity.

The steerable tubing 1310 can be composed with higher or lower density, higher or lower malleability, higher or lower flexibility, or other features for ease of traversing through the subject. The flexibility of the steerable tubing 1310 facilitates the navigation of the steerable instrument 1300 within the subject. The steerable tubing 1310 can be flexible as to not introduce injuries, tears, wounds, or other damages within the subject. The flexibility of the steerable tubing 1310 can allow the steerable instrument 1300 to articulate or rotate even while the steerable instrument 1300 is bent. For example, the flexible tubing of the steerable instrument 100 may be bent 120 degrees, including the components within the steerable instrument 100 such as the flexible torque component 1332. The bent steerable instrument can maintain the rotational performance with the flexibility of the flexible tubing at the 120 degrees bend. The steerable tubing 1310 can include any width or length. The width can be 1 millimeter, 2 millimeters, 3 millimeter, 4 millimeters, 5 millimeters, or 1 centimeter. The length can be 350 mm, 500 mm, 1 meters, 2 meters, 3 meters, 4 meters, 5 meters, 6 meters, 7 meters, 8 meters, 9 meters, 10 meters, 50 meters, or 100 meters.

In some embodiments, the steerable tubing 1310 can be inserted into an instrument channel or working channel or a surgical tool. The instrument channel can define a hollow portion or entrance configured for the steerable tubing 1310. The instrument channel can provide an additional shape, textures groove, or other features to the steerable tubing 1310, or provide a cover for traversing within the subject.

The steerable tubing 1310 can include or be coupled to one or more sensors, such as a light sensor, electromagnetic sensor, an optical stereotactic sensor, a pressure sensor, an impact sensor, a flow sensor, a radar sensor, a position sir, or a distance sensor. In some embodiments, the steerable tubing 1310 detects a presence of the materials. The steerable tubing 1310 can be equipped with at lit one sensor that can communicate with at least one external device, such as a sensor processing component (not shown) to determine the thickness of material relative the rest of the subject indicated by the sensor. The sensor can include, for example, a temperature sensor, a pressure sensor, a resistance sensor, an impact sensor, an ultrasonic sensor, or other sensor for medical examination. In some embodiments, the type of material is associated with at least an impedance or a density of the tissue. The sensor can gather temperature information and other sensed information, and provide signals corresponding to such information to the sensor-processing unit. The sensor-processing unit can subsequently identify the type of material. In some embodiments, the sensor can be an electrical sensor.

Referring now to FIG. 13A in conjunction with FIG. 13D, the steerable tubing 1310 can extend from the proximal end 1312 to the distal end 1314. The proximal end 1312 can refer to the base, the beginning, or the foundation of the steerable tubing 1310. The distal end 1314 can refer to the tip or the front of the steerable tubing 1310. The distal end 1314 earn be coupled to the outer sheath 1304. The steerable tubing 1310 can be configured to receive a torque (e.g., τ-proximate) at the proximal end 1312 and transmit the true to the distal end 1314 to the outer sheath 1304 (e.g., as τ-distal) to rotate the outer sheath 1304.

The steerable tubing 1310 includes the bate layer 1318 and the top layer 1320. The base layer 1318 and top layer 1320 may each include at least one of an elastomer or a friction reducing additive. In some implementations, the elastomer includes a thermoplastic elastomer such as polyester block amide (e.g., PEBAX). In some implementations, the friction reducing additive includes MOBILIZE, manufactured by Compounding Solutions, LLC of Lewiston, Me. The at least one of the elastomer or the friction reducing additive can reduce the likelihood of frictional losses during use that would reduce the efficiency of the steerable tubing 1310 in transmitting torque from the proximal end 1312 to the distal end 1314. In addition, the at least one of the elastomer or the friction reducing additive can reduce friction generated between the steerable tubing 1310 and the outer sheath 1304 when the steerable tubing 1310 and the outer sheath 1304 come in contact with one another, for example, when the steerable instrument 1300 has been passed through a tortuous pathway. In some embodiments in which the steerable instrument 1300 is insertable within a working channel of a surgical instrument, the at least one of the elastomer or the friction reducing additive can reduce friction generated between the steerable tubing 1310 and the inner wall of the working channel of the surgical instrument.

The steerable tubing 1310 defines an inner diameter 1322 and an outer diameter 1324. The inner diameter 1322 may be less than 2 mm. The inner diameter 1322 may be 1 mm. The inner diameter 1322 may be 10 mm. The inner diameter 1322 may be less than 0.5 inches. The inner diameter 1122 may be less than 0.25 inches. The inner diameter 1322 may be greater than or equal to 0.11 inches and less than or equal to 0.5 inches. The inner diameter 1322 may be greater than or equal to 0.11 inches and less than or equal to 0.13 inches. The outer diameter 1324 may be less than 4 mm. The outer diameter 1324 may be 5 mm. The outer diameter 1324 may be 10 mm. The outer diameter 1324 may be less than 0.5 inches. The outer diameter 1324 may be less than 0.25 inches. The outer diameter 1324 may be getter than or equal to 0.05 inches and less than or equal to 0.5 inches. The outer diameter 1324 may be greater than or equal to 0.11 inches and less than or equal to 0.13 inches. A ratio of the inner diameter 1322 to the outer diameter 1324 may be greater than or equal to 0.5 and less than or equal to 0.95.

The amble instrument 1300 can include the first connector 1326 for articulating the distal end 1314 of the steerable tubing 1310 along the longitudinal axis 1328 extending through the steerable instrument 1300 responsive to receiving a first control input at the first connector 1326. The flat connector 1326 can be coupled to the proximal end 1312 of the steerable tubing 1310. The first connector 1326 can be a knob, tube, handle, grip, or any other surface configured to receive control inputs from the operator. The control inputs can cause the first connector 1326 to bend the proximal end 1312 of the steerable tubing 1310 relative to the longitudinal axis 1328. For example, the control inputs can bend the proximal end 1312 of the steerable tubing 1310 by −90, −80, −70, −60, −50, −40, −30, −20, −10, 10, 20, 30, 40, 50, 60, 70, 80, or 90 degrees relative to the longitudinal axis 1328.

The first connector 1326 can be configured to articulate the distal end 1314 on the articulation axis 1330 relative to the longitudinal axis 1328. The articulation axis 1330 can be relative to the longitudinal axis 1328. For example, the articulation axis 1330 can be −90, −80, −70, −60, −50, −40, −30, −20, −10, 10, 20, 30, 40, 50, 60, 70, 80, or 90 degrees relative to the longitudinal axis 1328. The distal end 1314 can bend at an angle proportional to the angle bent by the proximal end 1312. For example, the steerable tubing 1310 can be configured to bend the distal end 1314 by 10 degrees responsive to a 10 degree bend of the proximal end 1312 by the first connector 1326. In another example, the steerable tubing 1310 is configured to bend according to any other configuration. For example, the steerable tubing 1310 can be configured to bend the distal end 1314 by 10 degrees responsive to a 10 degree bend of the proximal end 1312 by the first connector 1321, and bend the distal end 1314 by 15 degrees responsive to a 20 degree bend of the proximal end 1312 by the first connector 1326. In another example, the steerable tubing 1310 cart be configured to band the distal end 1314 by 10 degrees responsive to a 10 degree bend of the proximal end 1312 by the first connector 1326, and band the distal end 1314 by 25 degrees responsive to a 20 degree bend of the proximal end 1312 by the first connector 1326.

In some embodiments, the first connector 1326 can be configured to input the τ-proximal used to rotate the steerable tubing 1310 and thus the outer sheath 1304 for to apply a control torque corresponding to a desired τ-proximal, such as if the first connector 1326 includes gears and/or a motorised actuator to drive the rotation of the steerable tubing 1310). In some embodiments, the first connector 1326 is coupled to a motor configured to apply the torque or the control inputs. The distal end 1314 can rotate with an equivalent torque as the torque provided via the first connector 1326 at the proximal end 1312. It should be appreciated that the distal end 1314 can be configured to notate a specific degree equivalent or matching the degree of rotation of the proximal end 1312. Accordingly, the first connector 1326 can be configured to provide precision and control of the steerable tubing 1310 and thus the outer sheath 1304. For example, the operator may initiate a 30-degrees rotation of the first connector 1326. The rotation, force, and torque can be averted to the distal end 1314 such that the outer sheath 1304 also rotates by 30-degrees.

The steerable instrument 1300 can include the flexible torque component 1332 disposed within the steerable tubing 1310. The flexible torque component 1332 can be coupled to and disposed within the inner sheath 1306. In addition, at least one of the elastomer or the friction reducing additive can reduce friction generated between the flexible torque component 1332 and the inner sheath 1306 when the flexible torque component 1332 and the inner sheath 1306 come in contact with one another, for example, when the steerable instrument 1300 has been passed through a tortuous pathway. The flexible torque component 1332 can be configured to rotate the inner sheath 1306 relative to the outer sheath 1304 to cut the material. The flexible torque component 1332 can be composed of at least one of metal steel, plastic, titanium, nickel, carbon fiber, or other alloys. In some embodiments, the inner sheath 1306 can include a lining within which the flexible torque component 1332 is disposed.

The steerable instrument 1300 can include the second connector 1334 coupled to the proximal end 1312 of the steerable tubing 1310 and configured to rotate the flexible torque component 1332. The second connector 1334 can be coupled to the flexible torque component 1332. The second connector 1334 can be configured to receive control inputs from the operator. The second connector 1334 can be a knob, tube, handle, grip, or any other surface configured to receive control inputs.

The control inputs can rotate the second connector 1334 to cause the inner sheath 1306 to rotate relative to the outer sheath 1304 to cut the material. The control inputs can rotate the second connector 1334 any number of degrees. For example, the control inputs can rotate the second connector 1334 by 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, or 360-degrees relative to the longitudinal axis 1328. The inner sheath 1306 can rotate any number of degrees. For example, the inner sheath 1306 can rotate 10, 20, 30, 40, 50, 60, 70, 80, 90, 100, 110, 120, 130, 140, 150, 160, 170, 180, 190, 200, 210, 220, 230, 240, 251, 260, 270, 280, 290, 300, 310, 320, 330, 340, 350, or 360-degrees. The inner sheath 1306 can rotate at an angle proportional to the angle of rotation of the second connector 1334. For example, the inner sheath 1306 can be configured to rotate by 10 degrees responsive to a 10 degree rotation of the second connector 1334. In another example, the inner sheath 1306 is configured to bend according to any other configuration. For example, the inner sheath 1306 can be configured to rotate by 10 degrees responsive to a 10 degree rotation by the second connector 1334, but rotate by 15 degrees responsive to a 20 degree rotation by the second connector 1334. In another example, the inner sheath 1306 can be configured to rotate by 10 degrees responsive to a 10 degree rotation by the second connector 1334, but rotate by 25 degrees responsive to a 20 degree rotation by the second connector 1334.

The steerable instrument 1300 can include an aspiration channel 1336 extending from the cutting window 1308 to the aspiration port 1318. The aspiration channel 1336 can be partially defined by the flexible torque component 1332. The aspiration channel 1336 can be partially defined by an outer wall of the inner sheath 1306. The aspiration channel 1336 can be partially defined by an inner wall of the outer sheath 1304. Materials can enter the aspiration channel 1336 via the cutting window 1308 and traverse the length of the aspiration channel 1336 to the aspiration port 1338.

The aspiration port 1338 can be an opening or any other connection between the steerable tubing 1310 and a vacuum source 1340. The aspiration port 1338 can include sockets, plugs, or any other coupling mechanism configured to couple the steerable tubing 1310 and a vacuum source 1340. In some embodiments, the aspiration port 1338 can include additional tubing or hosing to couple the vacuum source 1340 to the steerable tubing 1310.

The vacuum source 1340 can retrieve, extract, or collect cut material from the aspiration channel 1336. The vacuum source 1340 can be configured to pull, draw, or drag the material. The vacuum source 1340 can be configured to initiate a suction feature or force to retrieve cut materials. The vacuum source 1340 can be configured to section liquid, fluid, or gas from the aspiration channel 1336. The aspiration channel 1336 can be configured to enable the vacuum source 1340 to maintain a suction force throughout the length of the aspiration channel 1336 by preventing air from escaping or entering through the aspiration channel 1336. The vacuum source 1340 can apply a vacuum pressure greater than or equal to 200 remits and less than or equal to 750 mmHg to retrieve the cut materials through the aspiration channel 1336. Accordingly, the vacuum source 1340 can be configured to aspirate, suction, or pull materials into aspiration channel 1336 for retrieval or extraction of the materials in some embodiments, vacuum source 1340 can include a collection cartridge or a repository for storing the cut materials or any other substance retrieved from the subject using the vacuum source 1340.

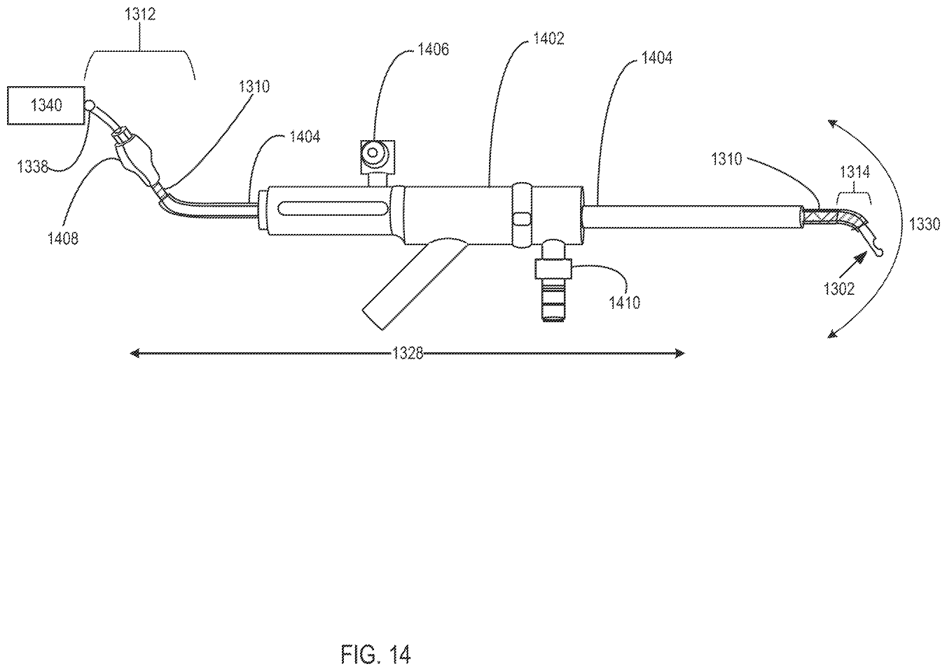

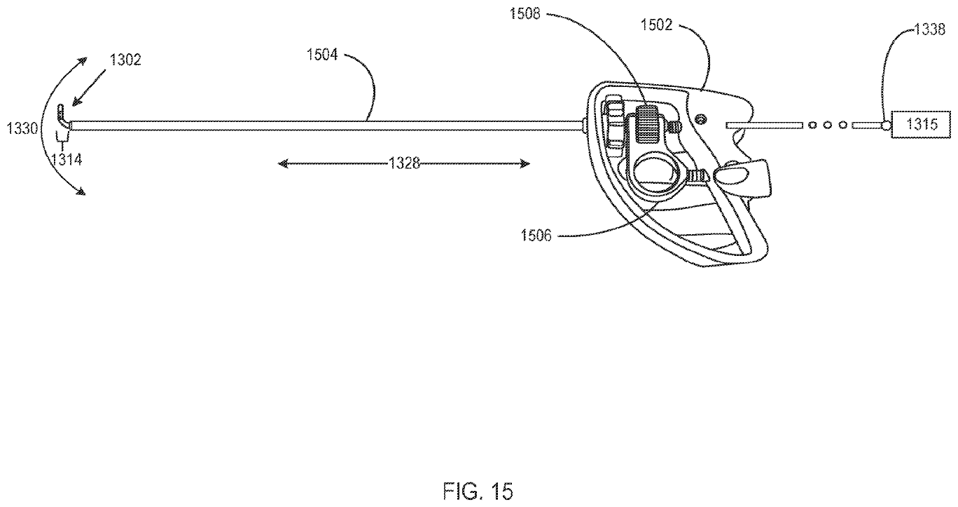

Referring to FIG. 14, shown is a view of a surgical tool 1402 for maneuvering the steerable instrument 1300 to a treatment site during a laparoscopic or hysteroscopic procedure according to embodiments of the present disclosure. The surgical tool 1402 can be inserted, situated, or resided in the subject. The surgical tool 1402 can be inserted into an opening or a cavity, such as those shown in FIGS. 1A-1D. The insertion of the surgical tool 1402 can be through the opening or the cavity of the subject. The surgical tool 1402 can be a flexible hysteroscope or laparoscope, such that the surgical tool 1402 can be turned, bent, or otherwise navigated through curvatures of the subject. In some embodiments, the surgical tool 1402 can introduce irrigation liquid. The irrigation liquid can flow along the steerable instrument 1300 to the treatment site.

While it is difficult to utilize the surgical instrument 1300 with the surgical tool 1402 in cavities and other narrow or tortuous treatment sites, the flexibility and small diameter of the surgical instrument 1300 can address this problem by enabling the surgical instrument 1300 to be inserted into the surgical tool 1402 through the tubing 1404. The instrument 1300 and the surgical tool 1402 can be navigated together to the treatment site, where the steerable instrument 1300 can articulate its distal end 1314 to maneuver the cutting assembly 1302 to the materiel. For example, the surgical instrument 1300 can cut the material while the surgical tool 1402 provides a camera or irrigation fluid.

The surgical tool 1402 can include tubing 1404 that defines a working channel or instrument channel. The length of the steerable instrument 1300 can be sized to exceed the length of the surgical tool 1402 and/or the tubing 1404. For example, the steerable instrument 1300 can be 100, 200, 150, 500, 750, or 900 mm longer than the surgical tool 1402 and/or the tubing 1404. The steerable instrument 1300 can extend any distance past a distal end of the surgical tool 1402. For example, the steerable instrument 1300 can extend 10, 20, 30, 40, 30, 60, 70, 80, 90, or 100 mm past the distal end of the surgical tool 1402. The steerable instrument 1300 can be sized, shaped or configured such that the outer diameter 1324 is less than the diameter of the channel in which the steerable instrument 1300 is to be inserted.

The surgical tool 1402 can include an irrigation entry port 1406. The irrigation entry port 1406 can be configured to introduce irrigation fluid into the surgical tool 1402. The irrigation entry pot 1406 can be configured to engage with an irrigation source, such as a saline or water container. In some implementations, the irrigation entry port 1406 can be a Y port used in fluid delivery systems that complies with medical device industry standards. The surgical tool 1402 can be configured such that the irrigation fluid flows between the outer wall of the surgical tool 1402 and the inner wall of the channel within the surgical tool 1402. The irrigation fluid can then be released at a distal end of the surgical tool 1402.

The steerable instrument 1300 can include a controller 1408 coupled to the steerable instrument 1300. The controller 1408 can be an embodiment and/or perform the functionality of the first connector 1326 and/or the second connector 1334. In some embodiments, the controller 1408 can be configured to receive a pushing or pulling force to maneuver the steerable instrument 1300 into the tubing 1404. In some embodiments, the pushing and pulling can cause the steerable instrument 1300 to expand or retract relative to the tubing 1404. In some embodiments, the controller 1408 can be configured to receive a pushing or pulling force to provide axial movement of the distal end 1314. The steerable tubing 1310 can be configured to receive axial movement at a proximal end 1312 such that there is axial movement at the distal end. In some embodiments, the controller 1408 can be configured to receive control inputs to maneuver the proximal end 1312 such that the distal end 1314 of the steerable tubing 1310 can maneuver along the articulation axis 1330. For example, the controller 1408 can be configured such that a degree bend relative to the longitudinal axis 1328 causes a 60-degree bend of the distal end 1314. In some embodiments, the controller 1408 can be configured to receive control inputs to rotate the flexible torque component 1332 to rotate the inner sheath 1306 to cut materials by the ding assembly 1302. For example the controller 1408 can be configured such that a 60-degree rotation causes the flexible torque component 1332 and the inner sheath 1306 to rotate by degree to cut the material. In some embodiments, the controller 1408 can be configured to receive torque to rotate the proximal end 1312 of the steerable tubing 1310 and the outer sheath 1304. For example, the steeple instrument 1300 can be configured such that a 360-degree rotation of the controller 1408 causes a 360-degree rotation of the proximal end 1312 and the distal end 1314 of the steerable tubing 1310, and thus the outer sheath 1304.

The surgical tool 1402 can include a light 1410 configured to illuminate the treatment site. The light 1410 can be a fiber optic light, a light emitting diode (“LED”), incandescent lamps, compact fluorescent, halogen, neon, or other types of lighting elements. In some embodiments, the controller 1408 can actuate the light 1410 to turn it on, off, or modulate its intensity.

Referring to FIG. 13, shown is a view of a surgical tool 1502 for maneuvering the steerable instrument 1300 to a treatment site during a laparoscopic or hysteroscopic procedure according to embodiments of the present disclosure. The surgical tool 1502 can be inserted, situated, or resided in the subject. The surgical tool 1502 can be inserted into an opening or a cavity, such as those shown in FIGS. 1A-1D. The insertion of the surgical tool 1502 can be through the opening or the cavity of the subject. The surgical tool 1502 can be a flexible hysteroscope or laparoscope, such that the surgical tool 1502 can be turned, bent, or otherwise navigated through curvatures of the subject.

While it is difficult to utilize the surgical instrument 1300 with the surgical tool 1502 in cavities and other narrow or tortuous treatment sites, the flexibility and small diameter of the surgical instrument 1300 can address this problem by enabling the surgical instrument 1300 to be inserted into the surgical tool 1502. The instruct 1300 and the surgical tool 1502 can be navigated together to the treatment site, where the steerable instrument 1300 can articulate its distal end 1314 to maneuver the cutting assembly 1302 to the material. For example, the surgical instrument 1300 can cut the material while the surgical tool 1502 provides a camera or irrigation fluid.

The surgical tool 1502 can include tubing 1504 that defines a working channel or instrument channel. The length of the stele instrument 1300 can be sized to exceed the length of the surgical tool 1502. The steerable instrument 1300 can extend any distance past a distal end of the surgical tool 1502. For example, the steerable instrument 1300 can extend 10, 20, 30, 40, 50, 60, 70, 80, 90, or 100 mm past the distal end of the surgical tool 1502. A length of the steerable instrument 1300 can exceed the length of the tubing 1504. For example, the length can be 100, 200, 350, 500, 750, or 900 mm.

The surgical tool 1502 can include a first connector 1506. The first connector 1506 can be coupled to the steerable instrument 1300. The first connector 1506 can be a lever, a trigger, or any other mechanism configured to receive control inputs from the operator. The first connector 1506 can be an embodiment and/or perform the functionality of the first connector 1326. The first connector 1506 and the second connector 1503 can be coupled to the steerable instrument 1300. In some embodiments, the first connector 1506 can be configured to receive a pushing or pulling force from the operator, and provide the pushing or pulling force to the steerable instrument 1300. In some embodiments, the pushing or pulling force presided by the first connector 1506 can provide axial movement or the distal end 1314. For example, the pushing or pulling force provided by the first connector 1326 can maneuver the distal end 1314 of the steerable tubing 1310 along the articulation axis 1330. For example, the first connector 1506 can be configured such than a first force causes a 30-degree bend of the distal end 1314, and a second force causes a 60-degree bend of the distal end 1314. The second force can be stranger than the first force. For example, the first force can be 5 N, and the second force can be 10 N.

The surgical tool 1502 can include a second connector 1508. The second connector 1508 can be coupled to the steerable instrument 1300. The first connector 1506 can be a wheel, a knob, or any other mechanism configured to receive control inputs from the operator. The second connector 1508 can be an embodiment and tar perform the functionality of the second connector 1334. In some embodiments, the second donee tar 1508 can be configured to receive control inputs to rotate the flexible torque component 1332 to rotate the inner sheath 1306 to cut materials by the cutting assembly 1302. For example, the second connector 1508 can be configured such that a 60-degree rotation causes the flexible torque component 1332 and the inner sheath 1306 to rotate by 60-degrees to cut the material. In some embodiments, the second connector 1508 can be configured to receive torque to rotate the proximal end 1312 of the steerable tubing 1310 and the outer sheath 1304. For example, the second connector 308 can be configured such that a 360-degree rotation causes a 360-degree rotation of the proximal end 1312 and the distal end 1314 of the steerable tubing 1310, and thus the outer sheath 1104.

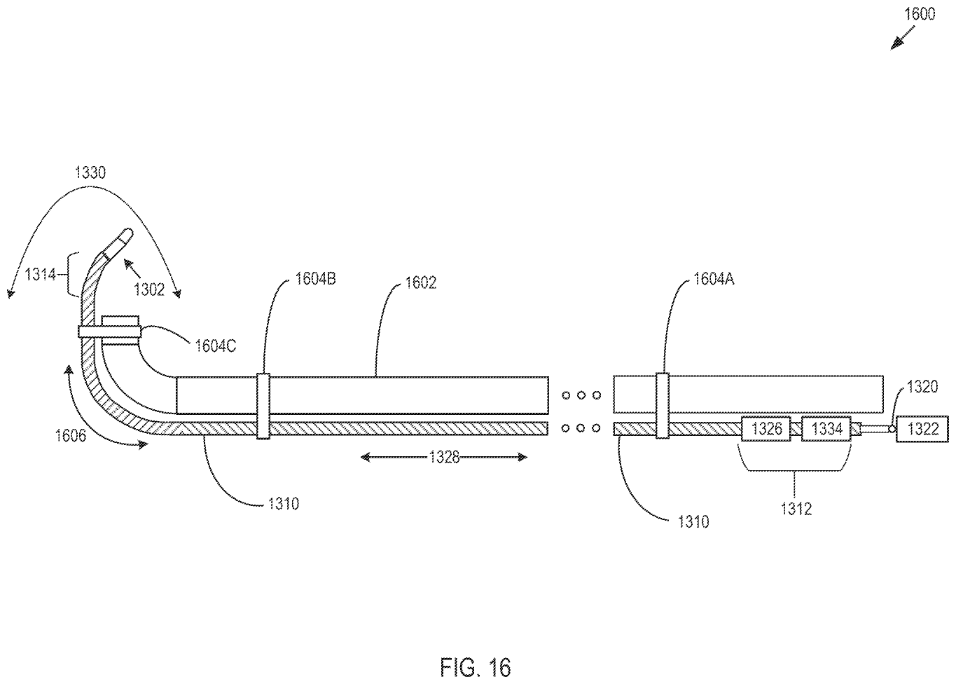

Referring to FIG. 16, shown is a view of a surgical tool 1602 for maneuvering the steerable instrument 1300 to a treatment she during a laparoscopic or hysteroscopic procedure according to embodiments of the present disclosure. The surgical tool 1602 can be inserted, situated, or resided in the subject. The surgical tool 1602 can be inserted into an opening or a cavity, such as those shown in FIG. 1A-1D. The insertion of the surgical tool 1602 can be through the opening or the cavity of the subject. The surgical tool 1602 can be a flexible hysteroscope or laparoscope, such that the surgical tool 1602 can turned, bent, or otherwise navigated through curvatures of the subject.

The surgical tool 1602 and the steerable instrument 1300 can be coupled by one or more attachment members 1604a-1604n (generally referred to as attachment members 1664). While it is difficult to utilize the surgical instrument 1300 with the surgical tool 1602 in cavities and other harrow or tortuous treatment sites, the attachment members 1604 can address this problem by enabling the surgical instrument 1300 to attach and navigate along an external side of the surgical tool 1602. The attachment members 1604 enable the surgical instrument 1300 and the surgical tool 1602 to be navigated together to the treatment site, where the steerable instrument 1300 can articulate its distal end 1314 to maneuver the cutting assembly 1302 to the material. For example, the instrument 1300 can cut the material while the surgical tool 1602 provides a camera, irrigation, or suction. In another example, the steerable instrument 1300 does not have or use the aspiration channel 1336 if the steerable instrument 1300 is utilized with the surgical tool 1602.

The attachment members 1604 can attach to the surgical tool 1602. The attachment members 1604 can be attached, disposed, or coupled along the lengths of the steerable tubing 1310 and the surgical tool 1602. The attachment members 1604 can include bands or fishing pole loops. Each of the attachment members 1604 can have an opening configured to receive the steerable instrument 1300. A diameter of the opening diameter can be less than 4 mm. The diameter can be 5 mm. The diameter may be 5.8 mm. The diameter can be 10 mm. The diameter can be less than 0.5 inches. The diameter can be less than 0.25 inches. The diameter may be greater than or equal to 0.05 inches and less than or equal to 0.5 inches. The diameter may be greater than or equal to 0.11 incites and less than or equal to 0.13 inches.

The length of the steerable instrument 1300 can be sized to exceed the length of the surgical tool 1602. The surgical tool 1602 can have an outside diameter 5.8 mm, an inside diameter of 4.5 mm, and a length 1200 mm. The steerable instrument 1300 can extend any distance past a distal end of the surgical tool 1602. For example, the steerable instrument ent 1300 can extend 10, 20, 30, 40, 30, 60, 70, 80, 90, or 100 mm past the distal end of the surgical tool 1602.

The attachment members 1604 can be composed of one or more substances, such as rubber, cloth, metal, steel, plastic, titanium, nickel, carbon fiber, or other alloys. The attachment members 1604 can include one or more textures or grooves, such as a spiral, a twist, frets, or other protrusion or engraving. The attachment members 1604 may be coated with at least one chemical compound for insertion into the subject, such as polymer, hydrophilic, nitinol, fluoropolymer, or a combination of two or more compounds to increased durability, lubrication, flexibility, or corrosion resistance of the attachment members 1604. The attachment members 1604 can be flexible as to not introduce injuries, tears, wounds, or other damages within the subject, to the steerable tubing 1310, or to the surgical tool 1602.

The steerable instrument 1300 can pass through and out of the attachment members 1604. For example, the proximal end 1312 of the steerable tubing 1310 can be configured to receive the pushing or pulling force from the operator, and provide the pushing car pulling force to the distal end 1314. The steerable tubing 1310 can rotate within the attachment members 1604. For example, the proximal end 1312 of steerable tubing 1310 can be configured to receive control inputs to rotate the steerable tubing 1310 and thus the steerable tubing 1310 to rotate the outer sheath 1304. For example, a 60-degree rotation of the proximal end 1312 can cause a 60-degree rotation of the distal end 1314.

The musical tool 1602 can bend along an axis 1606. For example, the axis 1606 can be an active bending section that is steerable to the treatment site. The axis 1606 can define an angle. For example, the angle can be −90, −80, −70, −60, −50, −40, −30, −20, −10, 10, 20, 30, 40, 50, 60, 70, 80, or 90 degrees relative to the longitudinal axis 1328. The bent portion of the surgical tool 1602 can define a radius. The radius can be 1, 2, 3, 4, 5, 6, 7, 8, 9, or 10 mm. The surgical tool 1602 can maneuver along the axis 1606 through each of the attachment members 1604. When the steerable instrument 1300 is secured to the surgical tool 1602 via the attachment members 1604, the first connector 1326 can articulate the distal end 1314 or rotate the outer sheath 1304, and the second connector 1334 can rotate the inner sheath 1306. The first connector 1326 and the second connector 1334 can articulate the distal end 1314 and rotate the outer sheath 1304 and the inner sheath 1306 regardless of the angle harmed by the axis 1606.

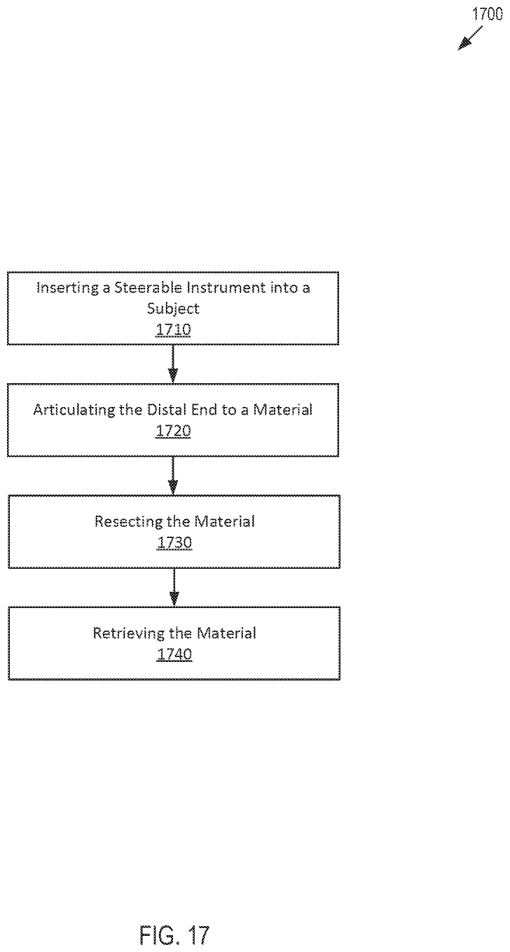

Referring to FIG. 17, a method 1700 of performing a laparoscopic or hysteroscopic procedure using the steerable instrument can be shown. The method 1700 can be performed using various embodiments of steerable instruments described herein. The method 1700 or steps thereof can be repeated, such as to address multiple treatment sites having multiple materials to be cut, both inside and outside the vessels.

At 1710, a steerable instrument (e.g., steerable instrument 1300) can be inserted into a subject. The steerable instrument can be disposed within a working channel of surgical instrument (e.g., surgical tool 1402, surgical tool 1502). The steerable instrument can include a cutting assembly (e.g., cutting assembly 1342) configured to cut the material. The cutting assembly can include an outer sheath (e.g., outer sheath 1304) and an inner sheath (e.g., inner sheath 1306) disposed within the outer sheath, the outer sheath defining a cutting window (e.g., cutting window 1308). The cutting assembly can be coupled to a flexible outer tubing (e.g., steerable tubing 1310). The flexible outer tubing can have an outer diameter (e.g., outer diameter 1324) less than 4 mm.

The cavity can be a body cavity or a spacing inside the body, such as the uterus, fallopian tubes, ovaries, mouth, the ear, the nose, the esophagus, etc. The cavity may be generated using at least one surgical procedure, such as a cut, a drill, or a dissection. The generated cavity can be in various different portions of the body of the subject, such as the uterus, arm, the stomach, the liver, the neck, etc.

The treatment site can include material to be cut from the subject. The material can include foreign material introduced into the subject, a solidified material clogging the passage of a vessel, or other material determined to be cut from the subject. The steerable instrument may include one or more sensors, light source, or other attachments to facilitate the movement or navigation of the steerable instrument towards the treatment site. The attachments can facilitate identification of the material at the treatment site, such as to receive visual feedback to indicate the material at the treatment site. The one of more sensors can include, for example, a tilt sensor, a proximity sensor, a light sensor, a pressure sensor, a flow sensor, an impact sensor, an ultrasound sensor, a distance sensor, or other sensors to facilitate an endoscopic procedure or operation. For example, a doctor or an operator may determine a location of the material using a non-intrusive imaging techniques, such as an x-ray, an ultrasound, or a computer tomography (“CT”) scan. In addition, the material can be located by navigating the steerable instrument to the treatment site and using the one or more sensors to identify the material, such as the camera or the light source. The steerable instrument may reach the treatment site or the material based on, for example, sensing a blockage within the vessel of the subject using the one or more sensor. The material may be identified using the camera of the steerable instilment and display an image on a display device external to the steerable instrument.

The procedure can include inserting a steerable instrument into the cavity of the subject. For example, a treatment site can be determined within a vessel of a subject, such as an artery, an arteriole, a capillary, a venule, or a vein. A cavity can be identified to lead to the vessel containing the treatment site. A doctor (or an operator) can insert the steerable instrument into the cavity leading to the vessel. The doctor can navigate the steerable instrument to the treatment site of the vessel. The steerable instrument can stop or terminate the navigation of the steerable instrument in response to reaching the treatment site. In some cases, the reached treatment site can be based on a camera inserted with or as a part of the steerable instrument. In some cases, the reached treatment site can be based on a length of the inserted steerable instrument. The length of the inserted steerable instrument can be determined based on a predetermined location of the treatment site via using x-ray, computer tomography (“CT”) scan, ultrasound, magnetic resonance imaging (“MRI”), or other non-intrusive imaging techniques.

In some embodiments, the steerable instrument may be inserted into the subject in conjunction with the surgical tool. In conjunction may refer to together with, at the same time as, or in an instance with, for example, the surgical tool. The operator or the doctor may insert the surgical tool, enclosing the steerable instrument, into the subject via the cavity. The steerable instrument may extend from the surgical tool such as through a hollow portion of the steerable instrument, and move deeper into the subject. The steerable instrument can move along the surgical tool to proceed deeper into the subject. For example at this point, the steerable instrument can be fixed in a location, a distance away from a distal end of the surgical tool, within the subject. The process may be repeated for the steerable instrument to reach or pass a treatment site to perform other laparoscopic or hysteroscopic procedures.

The steerable instrument may use the at least one sensor to navigate within the subject, determine a location of the material within the subject, and initiate the rotation of the cutting assembly in response to positioning the distal tube end of the steerable instrument at or near the treatment site. The positioning of the steerable instrument can refer to, for example, in contact with, 0.1 millimeter, 0.5 millimeter, 1 millimeter, or 1.5 millimeters from the material (or the treatment site).

The steerable instrument may be used to navigate or guide the material cutting device within the body of the subject along any tortuous path. Accordingly, the steerable instrument may determine the positioning of the steerable instrument to initiate a rotation for the cutting assembly to perform a cut, an extraction, or a debriding material within the subject. The determination to initiate the rotation can be based on, for example, a coupling or engagement between the cutting assembly and the steerable instrument.

The steerable instrument can pass the treatment site, such as through material located at the treatment site. The navigation or driving of the steerable instrument can be terminated in response to reaching, being in contact with, or passing the material or the treatment site. The laparoscopic or hysteroscopic procedure can include determining the location of a material or a treatment site. In some implementations, an operator may use at least one imaging tool to identify the location of the material, such as an x-ray, an MRI, or a CT scan. In some implementations, the operator or the doctor can utilize a camera or a scope to locate the material within the subject. The camera or the scope can be a part of the steerable instrument. For example, the operator may insert the steerable instrument twice to perform the material removal operation or procedure. Once for identifying the material, and the second to collect, extract, debride, or cut the material. In another example, the operator may insert the steerable instrument into the subject. The operator can navigate the steerable instrument within the subject to find the material. Once the material is found, the operator may initiate a rotation to the cutting assembly to debride and cut the cut materials. The process of debriding the material may be reforest to as removing the material. In this example, the material cutting device may be inserted once to complete the laparoscopic or hysteroscopic operation or procedure.

The extension of the steerable instrument can move towards a treatment site within the subject. The steerable instrument may extend or move pass the treatment site, in which an operator can terminate further extension of the steerable instrument into the subject. The steerable instrument can move towards the treatment site using the surgical tool. While moving towards the treatment site, the operator may push or exert a force to the proximal end of the steerable instrument. The steerable instrument can be moved further inside the subject and towards the treatment site in response to the force exerted to the proximal end.

In some embodiments, the steerable instrument or the surgical tool can provide or transmit at least one substance to a treatment site of a subject. The substance can include liquid, gas, or other chemical compounds. The substance may facilitate the process of debriding the material into the cut materials, for example, by exerted a gam substance to soften, disperse, or breakdown the material. Accordingly, the provision of the substance can assist the debriding process using the cutting assembly. In another example, the substance may assist in healing the subject, for example, by blocking a damaged portion of the vessel or by providing a medication to the treatment area within the vessel.

At 1720, the distal end (e.g., distal end 1314) of the steerable instrument can be articulated. A first control input can be applied to a first connector (e.g., first connector 1326) coupled to the proximal end of the flexible outer tubing to cause articulation of the distal end of the flexible outer tubing relative to a longitudinal axis extending through the steerable instrument. For example, the control inputs can bend the proximal end of the flexible outer tubing by −90, −80, −70, −60, −50, −40, −30, −20, −10, 10, 20, 30, 40, 50, 60, 70, 80, or 90 degrees relative to the longitudinal axis. The first control input can cause the distal end to twist and turn in various angles to maneuver the cutting assembly to the material.

The first connector can articulate the distal end on the articulation axis relative to the longitudinal axis. The articulation axis can be relative to the longitudinal axis. For example, the articulation axis can be −90, −80, −70, −60, −50, −40, −30, −20, −10, 10, 20, 30, 40, 50, 60, 70, 80, or 90 degrees relative to the longitudinal axis. The distal end can bend at an angle proportional to the angle bent by the proximal end. For example, the flexible outer tubing can bend the distal end by 10 degrees responsive to a 10 degree bend of the proximal end by the first connector. In another example, the flexible outer tubing can bend according to any other configuration. For example, the flexible outer tubing can bend the distal end by 10 degrees responsive to a 10 degree bend of the proximal end by the first connector, and bend the distal end by 15 degrees responsive to a 20 degree bend of the proximal end by the first connector. In another example, the flexible outer tubing can bend the distal end by 10 degrees responsive to a 10 degree bend of the proximal end by the first connector, and bend the distal end by 25 degrees responsive to a 20 degree bend of the proximal end by the first connector. The distal end of the steerable instrument may be positional a distance from the material, such as 1 millimeter, 1 inch, or 1 meter from the material.

In some embodiments, the control input can provide torque at the proximal end of the flexible outer tubing to rotate the outer sheath. For example, the control inputs can rotate the proximal end by 60-degrees to cause 60-degree rotation of the distal end. In some embodiments, the first connector can input the τ-proximal used to rotate the flexible outer tubing and thus the outer sheath tor to apply a control torque corresponding to a desired τ-proximal, such as if the first connector includes gears and/or a motorized actuator to drive the rotation of the flexible outer tubing). In some embodiments, the first connector receives the torque or the control inputs from a motor. The distal end can rotate with an equivalent torque as the torque provided via the first connector at the proximal end. In should be appreciated that the distal end can be configured to rotate a specific degree, equivalent or matching the degree of rotation of the proximal end. Accordingly, the first connector can provide precision and control of the flexible outer tubing and thus the outer sheath. For example the operator may initiate a 30-degrees rotation of the first connector. The rotation, force, and torque can be exceed to the distal end such that the outer sheath also rotates by 30-degrees.

At 1730, the cutting assembly can cut the material from the treatment site. A second control input can be applied to a second connector (e.g., second connector 1334) coupled to the proximal end of the flexible outer tubing to rotate a flexible torque component (e.g., flexible torque component 1332) disposed within the flexible outer tubing. In some cases, an operator may exert a manual or mechanism rotation as the second control input applied to the second connector.

The flexible torque component can be coupled to the inner sheath. The inner sheath can be rotated in response to receiving an exerted rotational force or torque from the second connector. The flexible torque component can cause the inner sheath to rotate relative to the outer sheath so cut the material. The removal of the material can refer so cutting, debriding, pulling, dissecting, or tearing she material limn the treatment site. The cutting assembly can cut the material in response to the rotation by the inner sheath. The torque can be provided by the second connector. The exerted rotation may traverse from the proximal end to the distal end of the steerable instrument. As an example, the second connector may provide 180 degrees rotation to the steerable instrument at a proximal end, and the distal end will rotate by 180 degrees.

At 1740, the material can be retrieved from the treatment site. The substances can include the cut materials, liquid, gas, or other chemical compounds within the body of the subject. The steerable instrument can include actuating a vacuum source (e.g., vacuum source 1340) coupled to the steerable instrument to provide suction through an aspiration channel (e.g., aspiration channel 1336) defined by an inner wall of the stele instrument to cut the material from the subject via the aspiration channel. The process of retrieving the cut materials may be concurrent to the debriding process by the cutting assembly. For example, the vacuum source can initiate a vacuum to withdraw the cut materials while the cutting assembly debrides the material. The cut materials can be stored in a container or a repository in the vacuum source and/or external to the steerable instrument.

In further example, responsive to the cutting assembly debriding a material into cut materials, the steerable instrument may pull in, withdraw, or pump out the cut materials from the subject. The cut materials tan be withdrawn via an aspiration channel. The process of providing the substance or withdrawing the cut material can be performed by a vacuum source. The vacuum source can be external to the steerable instrument. The vacuum source can be initiated by a signal or a mechanical trigger. A pump device may be connected to the aspiration channel configured to withdraw of the cut materials from the subject. The pump device may pull a substance from a repository and push the chemical into the subject. The pump device may withdraw the cut materials into a second repository for storage.

The steerable instrument may be cut from the subject upon or based on completion of the laparoscopic or hysteroscopic procedures or processes. The completion of the laparoscopic or hysteroscopic procedures can entail the debriding of the material, such as through an entire treatment site, or the collection of the debriding materials. For example, the treatment site can include a length of 3 inches. The steerable instrument may initiate a rotation of the cutting assembly and travel through the 3 inches or treatment site to debride the material. While debriding the material, the steerable instrument may retrieve or draw in the cut materials into the aspiration channel. For example, once the steerable instrument cut the material through the 3 inch length of the treatment site and retrieve the cut materials, the laparoscopic or hysteroscopic procedures may be completed.

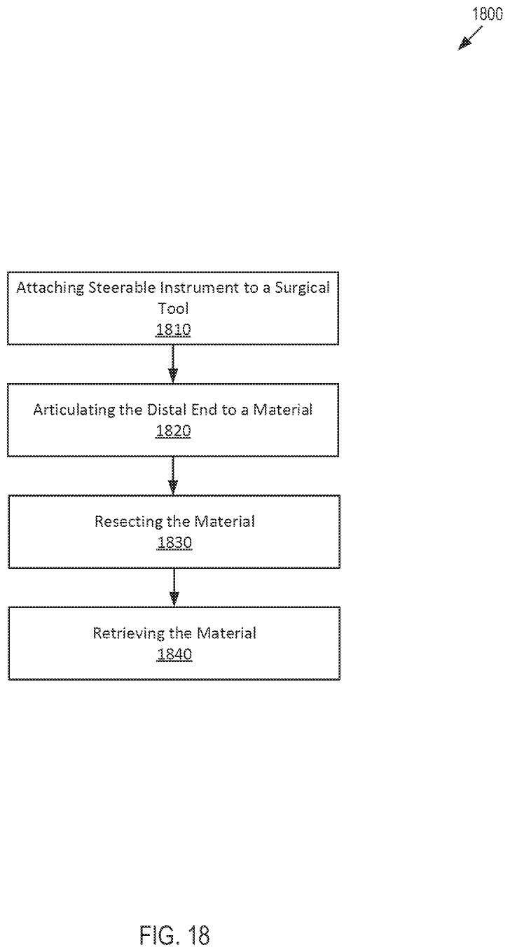

Referring to FIG. 18, a method 1800 of performing a laparoscopic or hysteroscopic procedure using the steerable instrument can be shown. The method 1800 can be performed using various embodiments of steerable instruments described herein. The method 1800 or steps thereof can be repeated, such as to address multiple treatment sites having multiple materials to be cut, both inside and outside the vessels.

At 1810, a steerable instrument (e.g., steerable instrument 1300) can be attached to a surgical tool (e.g. surgical tool 1602). The surgical tool and the attached steerable instrument can be inserted into a subject. The steerable instrument can include a cutting assembly (e.g., cutting assembly 1302) configured to cut the material. The cutting assembly can include an outer sheath (e.g., outer sheath 1304) and an inner sheath (e.g., inner sheath 1306) disposed within the outer sheath, the outer sheath defining a cutting window (e.g., cutting window 1308). The cutting assembly can be coupled to a flexible outer tubing (e.g., steerable tubing 1310). The steerable instrument and the surgical tool can be attached with attachment members (e.g., attachment members 1604). The attachment members can be positioned d along the surgical tool. The steerable instrument can be inserted into each attachment member. The steerable instrument can be maneuvered through each of the attachment members along the surgical tool to attach the steerable instrument to the surgical tool.

The cavity can be a body cavity or a spacing inside the body, such as the uterus, fallopian tubes, ovaries, mouth, the ear, the nose, the esophagus, etc. The cavity may be generated using at least one surgical procedure, such as a cut, a drill or a dissection. The generated cavity can be in various different portions of the body of the subject, such as the uterus, arm, the stomach, the liver, the neck, etc.

The treatment site can include material to be cut from the subject. The material can include foreign material introduced into the subject, a solidified material clogging the passage of a vessel, or other material determined to be cut from the subject. The steerable instrument may include one or more sensors, light source, or other attachments to facilitate the movement or navigation of the steerable instrument towards the treatment site. The attachments can facilitate identification of the material at the treatment site, such as to receive visual feedback to indicate the material at the treatment site. The one or more sensors can include, for example, a tilt sensor, a proximity sensor, a light sensor, a pressure sensor, a flow sensor, an impact sensor, an ultrasound sensor, a distance sensor, or other sensors to facilitate an endoscopic procedure or operation. For example, a doctor or an operator may determine a location of the material using a non-intrusive imaging techniques, such as an x-ray, an ultrasound, or a computer tomography (“CT”) scan. In addition, the material can be located by navigating the steerable instrument to the treatment site and using the one or more sensors to identify the material, such as the camera or the light source. The steerable instrument may teach the treatment site or the material based on, for example, sensing a blockage within the vessel of the subject using the one or more sensor. The material may be identified using the camera of steerable instrument and display an image on a display device external to the steerable instrument.

The procedure can include inserting a steerable instrument into the cavity of the subject, for example, a treatment site can be determined within a vessel of a subject, such as an artery, an arteriole, a capillary, a venule, or a vein. A cavity can be identified to lead to the vessel containing the treatment site. A doctor (or an operator) can insert the steerable instrument into the cavity leading to the vessel. The doctor can navigate the steerable instrument to the treatment site of the vessel. The steerable instrument can stop or terminate the navigation of the steerable instrument in response to reaching the treatment site. In some cases, the reached treatment site can be based on a camera inserted with or as a part of the steerable instrument. In some cases, the reached treatment site can be based on a length of the inserted steerable instrument. The length of the inserted steerable instrument can be determined based on a predetermined location of the treatment site via using x-ray, completer tomography (“CT”) scan, ultrasound, magnetic resonance imaging (“MRI”), or other non-intrusive imaging techniques.

In some embodiments, the steerable instrument may be inserted into the subject in conjunction with the surgical tool. In conjunction may refer to together with, at the same time as, or in an instance with, for example, the surgical toot. The operator or the doctor may insert the surgical tool, enclosing the steerable instrument, into the subject via the cavity. The steerable instrument may extend from the surgical tool, such as through a hollow portion of the steerable instrument, and move deeper into the subject. The steerable instrument can move along the surgical tool to proceed deeper into the subject. For example, at this point, the steerable instrument can be fixed in a location, a distance away from a distal end of the surgical tool, within the subject. The process may be repeated for the steerable instrument to reach or pass a treatment site to perform other laparoscopic or hysteroscopic procedures.

The steerable instrument may use the at least one sensor to navigate within the subject, determine a location of the mated within the subject, and initiate the rotation of the cutting assembly in response to positioning the distal tube end of the steerable instrument at or neat the treatment site. The positioning of the steerable instrument can refer to, for example, its contact with, 0.1 millimeter, 0.5 millimeter, 1 millimeter, or 1.5 millimeters from the material for the treatment site).

The steerable instrument may be used to navigate or guide the material cutting device within the body of the subject along any tortuous path. Accordingly, the steerable instrument may determine the positioning of the steerable instrument to initiate a mutation for the cutting assembly to perform a cut, an extraction, or a debriding of a material within the subject. The determination to initiate the rotation can be based on, for example, a coupling or engagement between the cutting assembly and the steerable instrument.

The steerable instrument can pass the treatment site, such as through material located at the treatment site. The navigation or driving of the steerable instrument can be terminated in response to reaching, being in contact with, or passing the material or the treatment site. The laparoscopic or hysteroscopic lure can include determining the location of a material or a treatment site. In some implementations, an operator may use at least one imaging tool to identify the location of the material, such as an x-ray, an MRI, or a CT scan. In some implementations the operator or the doctor can utilize a camera or a scope to locate the material within the subject. The camera or the scope can be a part of the steerable instrument. For example, the operator may insert the steerable instrument twice to perform the material removal operation or procedure. Once for identifying the material, and the second to collect, extract, debride, or cut the material. In another example, the operator may amen the steerable instrument into the subject. The operator can navigate the steerable instrument within the subject to find the material. Once the material is found, the operator may initiate a rotation to the cutting assembly to debride and cut the cut materials. The process of debriding the material may be retested to as removing the material. In this example, the material cutting device may be inserted once to complete the laparoscopic or hysteroscopic operation or procedure.

The extension of the steerable instrument can move towards a treatment site within the subject. The steerable instrument may extend or move pass the treatment site, in which an operator can terminate further extension of the steerable instrument into the subject. The steerable instrument can move towards the treatment site using the surgical tool. While moving towards the treatment site, the operator may push or exert a force to the proximal end of the steerable instrument. The steerable instrument can be moved further inside the subject and towards the treatment site in response to the force exerted to the proximal end.

In some embodiments, the steerable instrument or the surgical tool can provide or transmit at least one substance to a treatment site of a subject. The substance can include liquid, gas, or other chemical compounds. The substance may facilitate the process of debriding the material into the materials, for example, by exerted a gaseous substance to soften, disperse, or breakdown the material. Accordingly, the provision of the substance can assist the debriding process using the cutting assembly. In another example, the substance may assist in healing the subject, for example, by blocking a damaged portion of the vessel or by providing a medication to the treatment area within the vessel.

At 1820, the distal end (e.g., distal end 1314) of the steerable instrument can be articulated. A first control input can be applied to a first connector (e.g., first connector 1326) coupled to the proximal end of the flexible outer tubing to cause articulation of the distal end of the flexible outer tubing relative to a longitudinal axis extending through the steerable instrument. For example, the control inputs can bend the proximal end of the flexible outer tubing by −90, −80, −70, −60, −50, −40, −30, −20, −10, 10, 20, 30, 40, 50, 60, 70, 80, or 90 degrees relative to the longitudinal axis. The first control input can cause the distal end to twist and turn in various angles to maneuver the cutting assembly to the material.

The first connector can articulate the distal end on the articulation axis relative to the longitudinal axis. The articulation axis can be relative to the longitudinal axis. For example, the articulation axis can be −90, −80, −70, −60, −50, −40, −30, −20, −10, 10, 20, 30, 40, 50, 60, 70, 80, or 90 degrees relative to the longitudinal axis. The distal end can bend at an angle proportional to the angle bent by the proximal end. For example, the flexible outer tubing can bend the distal end by 10 degrees responsive to a 10-degree bend of the proximal end by the first connector. In another example, the flexible outer tubing can bend according to any other configuration. For example, the flexible outer tubing can bend the distal end by 10 degrees responsive to a 10-degree bend of the proximal end by the first connector, and bend the distal end by 15 degrees responsive to a 20-degree bend of the proximal end by the first connector. In another example, the flexible outer tubing can bend the distal end by 10 degrees responsive to a 10-degree bend of the maximal end by the first connector, and bend the dial end by 25 degrees responsive to a 20-degree bend or the proximal end by the first connector. The distal end of the steerable instrument may be positioned a distance fora the material, such as 1 millimeter, 1 inch, or 1 meter from the material.

In some embodiments, the control input can provide torque at the proximal end of the flexible outer tubing to rotate the outer sheath. For example, the control inputs can rotate the proximal end by 60-degrees to cause 60-degree rotation of the distal end. In some embodiments, the first connector can input the τ-proximal used to rotate the flexible outer tubing and thus the outer sheath (or to apply a control torque corresponding to a desired τ-proximal, such as if the first connector includes gears and/or a motorized actuator to drive the rotation of the flexible outer tubing). In some embodiments, the first connector receives the torque or the control inputs from a motor. The distal end can rotate with an equivalent torque as the torque provided via the first connector at the proximal end. It should be appreciated that the distal end 1314 can be configured to rotate a specific degree, equivalent or matching the degree of rotation of the proximal end. Accordingly, the first connector can provide precision and control of the flexible outer tubing and thus the outer sheath. For example, the operator may initiate a 30-degrees rotation of the first connector. The rotation, force, and torque can be exerted to the distal end such that the outer sheath also rotates by 30-degrees.

At 1830, the cutting assembly can cut the material from the treatment site. A second control input can be applied to a second connector (e.g., second connector 1334) coupled to the proximal end of the flexible outer tubing to rotate a flexible torque component (e.g., flexible torque component 1332) disposed within the flexible outer tubing. In some case, an operator may exert a manual or mechanism rotation as the second control input applied to the second connector.

The flexible torque component can be coupled to the inner sheath. The inner sheath may rotate in response to receiving an exerted rotational force or torque from the second connector. The flexible torque component can be configured to rotate the inner sheath relative to the outer sheath to cut the material. The removal of the material can refer to cutting, debriding, pulling, dissecting, or tearing the material from the treatment site. The cutting assembly can cut the material in response to the rotation by the inner sheath. The torque can be provided by the second connector. The exerted rotation may traverse from the proximal end to the distal end of the steerable instrument. As an example, the second connector may provide 180 degrees rotation to the steerable instrument at a proximal end, and the distal end will rotate by 180 degrees.

At 1840, the material can be retrieved from the treatment site. The substances can include the cut materials, liquid gas, or other chemical compounds within the body of the subject. The steerable instrument can include actuating a vacuum source (e.g., vacuum source 1340) coupled to the steerable instrument to provide suction through an aspiration channel (e.g., aspiration channel 1336) defined by an inner wall of the steerable instrument to cut the material from the subject via the aspiration channel. The process of retrieving the cut materials may be concurrent to the debriding process by the cutting assembly. For example, the vacuum source can initiate a vacuum to withdraw the cut materials while the cutting assembly debrides the material. The cut materials can be stored in a container or a repository in the vacuum source and/or external to the steerable instrument.

In further example, responsive to the cutting assembly debriding a material into cut materials, the vacuum source may pull in, withdraw, or pump out the cut materials from the subject. The cut materials can be withdrawn via an aspiration channel. The process of withdrawing the cut material can be performed by a vacuum source. The vacuum source can be external to the steerable instrument. The vacuum source device can be initiated by a signal or a mechanical trigger. A pump device may be connected to the aspiration channel configured to withdraw of the cut materials from the subject. The pump device may pull a substance from a repository and push the chemical into the subject. The pump device may withdraw the cut materials into a second repository for storage.

The steerable instrument may be cut from the subject upon or based on completion of the laparoscopic can hysteroscopic procedures was or processes. The completion of the laparoscopic or hysteroscopic procedures can entail the debriding of the material such as through an entire treatment site, or the collection of the debriding materials. For example, the treatment site can include a length of 3 inches. The steerable instrument may initiate a rotation of the cutting assembly and travel through the 3 inches of the treatment site to debride the material. While debriding the material, the steerable instrument may retrieve or draw in the cut materials into the aspiration channel. For example, once the steerable instrument cut the material through the 3 inch length of the treatment site and retrieve the cut materials, the laparoscopic or hysteroscopic procedures may be completed.

### E. Systems and Methods for a Steerable Overtube Instrument or Maneuvering to a Treatment Site

It is difficult to maneuver a cutting assembly at a distal end of a surgical instrument to a desired material at a treatment site while retaining the ability of the cutting assembly at the distal end of the surgical instrument to be properly operated, and even more difficult to use surgical instrument with other surgical tools in cavities and other narrow or tortuous treatment sites. A steerable instrument and methods thereof in accordance with the present disclosure can enable independent articulation of a distal end of the steerable instrument while retaining the ability of a cutting assembly at the distal end of the steerable instrument to be properly operated. The steerable overtube can enclose existing surgical instruments to provide the unique articulation described herein. The flexibility and small diameter of the steerable instrument enables the steerable instrument to navigate through a cavity or working channel of the surgical tool. For surgical tools that cannot receive the steerable instrument through their working channel, the attachment members enable the steerable instrument to navigate or along the external side of the surgical tool. While the steerable instrument is disposed in the cavity, the working channel, or the attachment members along the flexible tool, the steerable instrument can then articulate the distal end and actuate the cutting assembly thereof without articulating the rest of the steerable instrument to avoid damage or kinks to the cavity, surgical tool, or the steerable instrument itself.

The steerable instrument can include components such as a cutting assembly, a flexible tubing, a first connector, a flexible torque component, a second connector, and an aspiration channel. Generally, the steerable instrument may be used to provide treatment in narrow portions of a body, such as a uterus, fallopian tubes, ovaries, or in some cases, to provide non-surgical treatment to a subject. The steerable instrument may be guided to a treatment site to perform a laparascopic or hysteroscopic procedure. For example, the operator may insert the steerable instrument into a cavity of the subject and articulate the cutting assembly to a treatment site. In some embodiments, the operator inserts the steerable instrument into a channel of a steerable instrument. In other embodiments, the steerable instrument includes at least one attachment member configured to attach the steerable instrument along the steerable instrument.

After the steerable instrument is at the treatment site, the operator can steer the cutting assembly to the material. The location of the material can refer to a treatment site, portion, or area for extraction, inspection, or performing other procedures using the steerable instrument. The cutting assembly can be configured to cut the material and includes an outer sheath and an inner sheath disposed within the outer sheath. The steerable instrument can include a steerable tubing that articulates at a distal end responsive to inputs at a proximal end. The steerable instrument can include a first connector configured to articulate the steerable tubing along a longitudinal axis extending through the steerable instrument. The steerable instrument can include a flexible torque component configured to rotate the inner sheath relative to the outer sheath to cut the material. The steerable instrument can include a second connector configured to rotate the flexible torque component to cause the cutting assembly to cut the material. The steerable instrument can include an aspiration channel connected to a vacuum source configured to suction the material cut by the cutting assembly.

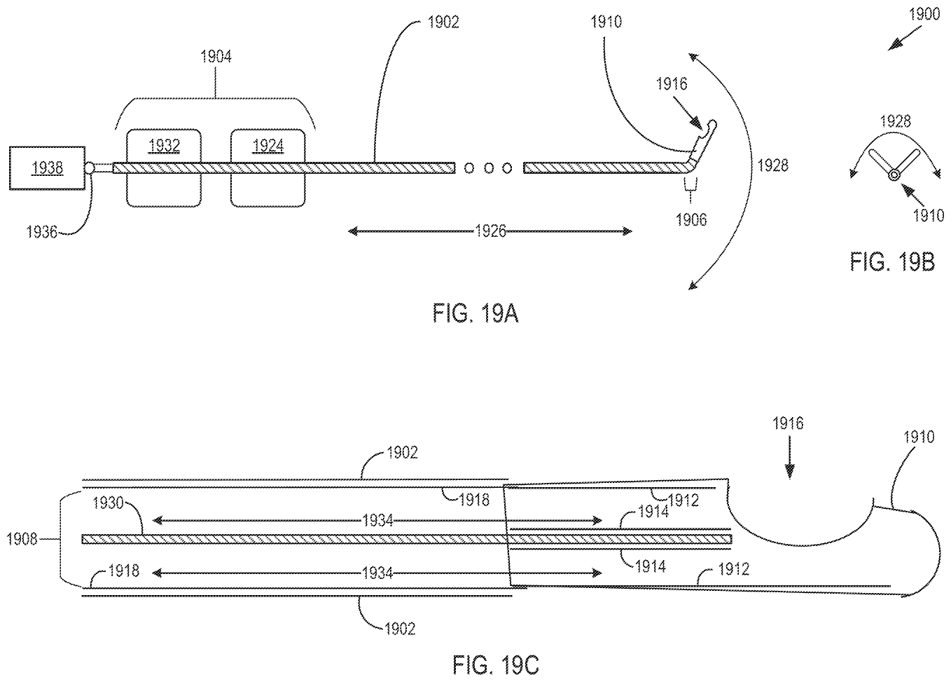

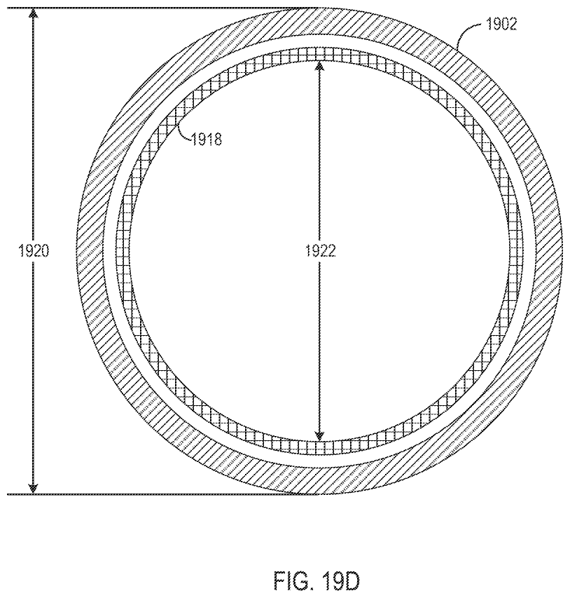

Referring to FIGS. 19A-19D, shown are views of the steerable instrument 1900 for maneuvering a cutting assembly to a treatment site during a laparoscopic or hysteroscopic procedure according to embodiments of the present disclosure. The steerable instrument 1900 can include steerable tubing 1902 extending from a proximal end 1904 to a distal end 1906. The steerable tubing 1902 can include or enclose a surgical instrument 1908. By enclosing the surgical instrument 1908, the steerable tubing 1902 enables the surgical instrument 1908 to have the unique articulation described herein, such that the distal end 1906 and the cutting assembly 1910 can be articulated to the material while retaining the ability of the cutting assembly 1910 to be properly operated.

The surgical instrument 1908 can include a cutting assembly 1910, which can include an outer sheath 1912 and an inner sheath 1914. The outer sheath 1912 can define a cutting window 1916. The surgical instrument 1908 can include a flexible tubing 1918. The steerable tubing 1902 can include a steerable tubing diameter 1920, and the flexible tubing 1918 can include a flexible tubing diameter 1922. The surgical instrument 1908 can include a first connector 1924, a longitudinal axis 1926, an articulation axis 1928, a flexible torque component. 1930, a second connector 1932, an aspiration channel 1934, and an aspiration port 1936 configured to couple to a vacuum source 1938.

For example, referring further to FIG. 19A, for performing a procedure to cut material from the treatment site, the steerable instrument 1900 can be introduced into a cavity of the subject. The steerable instrument 1900 can be maneuvered within the subject. The operator can use the first connector 1924 to maneuver the cutting assembly 1910 along the articulation axis 1928 to the material. The operator can use the second connector 1932 to actuate the cutting assembly 1910 to cut the material. A motor can also initiate a rotation to estate the cutting assembly 1910. The cutting assembly 1910 can rotate in response to the initiated rotation by the second connector 1932 or the motor. The material may be extracted, cut, collected, or investigated by the steerable instrument 1900. In some cases, the cutting assembly 1910 can extract, pull, or collect the material into the cutting window 1916. The vacuum source 1938 can suction the material into the aspiration channel 1934 extending from the cutting window 1916 to the aspiration port 1936.

In some embodiments, the steerable instrument 1900 can be inserted into an instrument channel or working channel of a surgical tool. The instrument channel can define a hollow portion or entrance configured for the steerable instrument 1900. The instrument channel can provide an additional shape, texture, groove, or other features to the flexible tubing 1918, or provide a cover for traversing within the subject.

The steerable tubing 1902 can be turned, bent, or otherwise navigated through curvatures of the subject. The treatment site can be located past the non-linear path within the subject. For example, the bodily cavity can include curves, bumps, or otherwise non-linear paths to a treatment site. The steerable instrument 1900 can be in contact with the subject, such that the steerable tubing 1902 can navigate through the curved portion of the subject. The flexible tubing 1918 can push, bump, or impact within the bodily cavity to turn through the non-linear path of the cavity. The steerable tubing 1902 can be bent or a turned in response to reaching or being in contact with the curved portion, such that the steerable tubing 1902 curves through the curved portion while navigating. In some cases, the flexible tubing 1918 can be navigated through a cavity by bouncing, turning, or adjusting a navigation direction in response to at least a contact with the cavity. The steerable tubing 1902 can be composed with higher or lower density, higher or lower malleability, higher or lower flexibility, or other features for ease of traversing through the subject. The flexibility of the steerable tubing 1902 facilitates the navigation of the steerable instrument 1900 within the subject. The steerable tubing 1902 can be flexible as to nor introduce injuries, tears, wounds, or other damages within the subject. The flexibility of the steerable tubing 1902 can allow the steerable tubing 1902 to articulate or rotate even while the steerable tubing 1902 is bent. For example, the steerable tubing 1902 may be bent 120 degrees, including the components within the steerable tubing 1902 such as the flexible tubing 1918. The bent steerable tubing 1902 can maintain the rotational performance with the flexibility of the flexible tubing at the 120 degrees bend.

The steerable tubing 1902 can be a navigation wire, a motorized wire, or a braid. The steerable tubing 1902 can include nitinol or other memory material such that articulation of the proximal end 1904 would cause articulation orate distal end 1906. The steerable tubing 1902 can include rubber, cloth, metal, steel, plastic, titanium, nickel, or carbon fiber. The steerable tubing 1902 can be a braided sheath. The steerable tubing 1902 can include any width or length. The width can be 1 millimeter, 2 millimeters, 3 millimeter, 4 millimeters, 5 millimeters, or 1 centimeter. The length tan be 350 mm, 500 mm, 1 meters, 2 meters, 3 meters, 4 meters, 5 meters, 6 meters, 7 meters, 8 meters, 9 meters, 10 meters, 30 meters, or 100 meters. The settable tubing 1902 can have a bending radius. The bending radius can be 5, 10, 15, 20, 25, 30, 35, 40, 45, or 50 mm.

The steerable tubing 1902 can extend from the proximal end 1904 to the distal end 1906. The proximal end 1904 can refer to the base, the beginning, or the foundation of the steerable tubing 1902. The distal end 1906 can refer to the tip or the front of the steerable tubing 1902. The steerable tubing 1902 can be configured to receive a torque at the proximal end 1904 to cause the distal end 1906 to articulate. The distal end 1906 can be configured to articulate in any direction. For example, the distal end 1906 can bend at an angle of 10, 20, 30, 40, 50, 60, 70, 80, or 90 degrees. The distal end 1906 can support a force during articulation. For example, the distal end 1906 can support 1, 2, 3, 4, 5, 6, 7, 8, 9, or 10 N. The steerable mixing 1902 can include the surgical instrument 1908.

Referring to FIG. 19A in conjunction with FIG. 19B, the cutting assembly 1910 can be configured to cut material from a subject. The cutting assembly 1910 can be coupled to or located at the distal end of the surgical instrument 1908. The cutting assembly 1910 can be a distance from the distal end 1906 of the steerable tubing 1902. For example, the distance can be 1, 2, 3, 4, 5, 6, 7, 8, 9, or 10 millimeters. The cutting assembly 1910 can include the blade (or a fan blade). The cutting assembly 1910 can include one or more blades, such as two blades as shown in FIG. 19B. The cutting assembly 1910 can include a fan, an axial cutter, a drill, a book, a scoop, a reamer, a miller cutter, or other cutting tools or devices. The cutting assembly 1910 can be referred to as a debriding component, a cutter, a removal tool, or an extractor. The cutting assembly 1910 can include a blade. The blade can be composed of one or more materials for cutting or dissecting a material, such as a steels, plastics, carbon fibers, titanium, aluminum, metals, or other alloys for performing laparoscopy or hysteroscopy operations. The cutting assembly 1910 can perform actions, including but not limited to, cutting, snaring, shredding, slicing, shattering, either entirely or partially, are also examples of deriding. Accordingly, the cutting assembly 1910 may be a component that is capable of cutting, snaring, shredding, slicing, or shattering from a surface of the body of the subject. As such, the cutting assembly 1910 may be implemented as a forceps, scissor, knife, snare, shredder, or any other component that can debride.

The cutting assembly 1910 may be actuated such that the cutting assembly 1910 may be operated through the translation of mechanical forces exerted by an operator or automatically actuated, using a turbine, a motor (e.g., electrical motor), or any other force generating component to actuate the debriding component. The cutting assembly 1910 can be configured to cut at various speeds, such as 5000 rotations per minute (“RPM”), 10,000 RPM, 20,000 RPM, or 50,000 RPM. The cutting assembly 1910 may be manually operated or may utilize any other means of debriding material such that the cut material are capable of being retried from the treatment site via the surgical instrument 1908. The cutting assembly 1910 can cut the material into small enough pieces, which may be retrieved via the surgical instrument 1908 such that the steerable instrument 1900 does not need to be cut from the subject to collect the cut material. It should be appreciated that using a cutting assembly 1910 that is able to rotate a specific degrees and with a specific torque, equivalent or matching the rotation and torque of the motor or operator. Accordingly, the cutting assembly 1910 can provide cutting precision, control, and power consumption. For example, the cutting assembly 1910, coupled to the cutting assembly 1910, can rotate a number of degrees with a specific torque equivalent to an operator providing the degrees and torque to the motor. For example, the operator or motor may initiate a 30-degrees rotation. The rotation, force, and torque can be exerted from the nor to the cutting assembly 1910. The tuning assembly 1910 can receive the exerted rotation. Accordingly, the cutting assembly 1910 may rotate 30-degrees based on the exerted rotation, force, and torque of the motor or operator.

The cutting assembly 1910 can include at least one sensor, such as a proximity sensor, a light sensor, a pressure sensor, a radar sensor, a flow sensor, a flex sensor, an impact sensor, a distance sensor, or other sensor configured to inspect, examine, sense, or navigate through a body of a subject. The cutting assembly 1910 may include a night source and a recording device or capturing device (e.g., a camera or a scope) to collect visual information from an invective of the body of the subject. The light source can include a light emitting diode (“LED”), incandescent lumps, compact fluorescent, halogen, neon, or other types of lighting elements. The surgical tool or the cutting assembly may emit light and initiate recording using the light source and the recording device. The cutting assembly 1910 may receive at least one visual information horn the camera and transmit the at least one visual information to the display device. The display device can generate or display the images based on the received visual information for an operator or a doctor to view inside the body of the subject during an operation. In some embodiments, the cutting assembly 1910 can be equipped with an injectable dye component through which the operator can use to determine the extent of narrowing under fluoroscopic guidance or to mark a particular region within the subject. In other embodiment, the operator can mark a particular region with the cutting assembly 1910, without the use of an injectable dye.

Referring to FIG. 19A in conjunction with FIG. 19C, the cutting assembly 1910 can include the outer sheath 1912 and the inner sheath 1914 disposed within the outer sheath 1912. The outer sheath 1912 can be a cover, an outer tube, a shell, or a main body of the cutting assembly 1910. The outer sheath 1912 can be shaped or formed to, for example, a cylinder, a prism, a cone, or other shapes. The outer sheath 1912 can be flexible. The outer sheath 1912 can bend and flex to any degree. In wine embodiments, the outer sheath 1912 can bend and flex to 10, 20, 30, 40, 50, 60, 70, 80, 90, 100, 110, 120, 130, 140, 150, 160, 170, or 180 degrees. The outer sheath 1912 can include a thickness. The thickness can be 10 nanometers, 20 nanometers, 1 millimeter, 2 millimeters, 3 millimeter, 4 millimeters, or 5 millimeters. The outer sheath 1912 can include a width. The width can be 1 millimeter, 2 millimeters, 3 millimeter, 4 millimeters, 5 millimeters, or 1 centimeter. The outer sheath 1912 can include a length. The length can be 1 meter, 2 meters, 3 meters, 4 meters, 5 meters, 6 meters, 7 meters, 8 meters, 9 meters, 10 meters, 50 meters, 100 meters, etc. The outer sheath 1912 can include a cross-sectional am, such as 0.6 millimeters squared, 1 millimeters squared, 1.9 millimeters squared, etc. The outer sheath 1912 can be composed of materials, such as metal, steel, plastic, rubber, glass, carbon fiber, titanium, aluminum, or other alloys.

The outer sheath 1912 can at least partially surround the inner sheath 1914. In some embodiments, the inner sheath 1914 cuts any material suctioned into or otherwise entering the outer sheath 1912. The inner sheath 1914 can include an opening such that material cut by the cutting assembly 1910 enters via the opening. The inner sheath 1914 can include a length similar to or less than the outer sheath 1912. The length can be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 35, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 cm. The inner sheath 1914 can be designed to facilitate debriding one or more materials and removing the cut materials in a single operation. The inner sheath 1914 can be disposed within the outer sheath 1912. The inner sheath 1914 can couple with the outer sheath 1912. The inner sheath 1914 can be composed of a similar maternal as the outer sheath 1912. The inner sheath 1914 can be flexible, similar to the outer sheath 1912.

The outer sheath 1912 can define the cutting window 1916. The outer sheath 1912 can include the cutting window 1916, at a distal end of the cutting assembly 1910. A portion of the radial wall of the outer sheath 1912 can define the cutting window 1916 that extends around a portion of the radius of the outer sheath 1912. In some embodiments, the operator can receive or retrieve cut materials through the cutting window 1916.

The cutting window 1916 can be configured to enable the cutting assembly 1910 to cut, dissect, or debride the material. For example, the cutting assembly 1910 can initiate the debriding or cutting process by rotating the cutting through the material to receive the material in the cutting window 1916. The cutting window 1916 can be positioned at a side of the cutting assembly 1910. The cutting window 1916 can be configured to enable tangential or side cutting of material with respect to the movement of the cutting assembly 1910. In some embodiments, the outer sheath 1912 can include the cutting window 1916. The cutting window 1916 can include a hollow structure with a shape, such as a circle, an oval, a rectangle, or other geometric shape for expositing the blades of the cutting assembly 1410. The cutting window 1916 can include a diameter. The diameter can be 1 millimeter, 2 millimeters, 3 millimeters, 4 millimeters, or 3 millimeters. The cutting window 1916 can include a cut out, which can be a portion of the cutting assembly 1910. For example, the cutting window 1916 can include a 0.4 millimeters cut out.

The flexible tubing 1918 can be a tube, a motorized wire, or a braid. The flexible tubing 1918 can include rubber, cloth, metal, steel, plastic, titanium, nickel, or carbon fiber. The flexible tubing 1918 can be a braided sheath. The flexible tubing 1918 can include any length. The length can be 350 mm, 500 mm, 1 meters, 2 meters, 3 meters, 4 meters, 5 meters, 6 meters, 7 meters, 8 meters, 9 meters, 10 meters 50 meters, or 100 meters. The flexibility of the flexible tubing 1918 can allow the flexible tubing 1918 to articulate or rotate even while the flexible tubing 1918 is bent. The steerable tubing 1902 within which the flexible tubing 1918 is disposed, and the components within the flexible tubing 1918 such as the flexible torque component 1930 can also bend with the flexible tubing 1918. For example, the flexible tubing 1918 may be bent 120 degrees. The bent flexible tubing 1918 can maintain the rotational performance with the flexibility of the flexible tubing 1918 at the 120 degrees bend.

In some embodiments, the flexible tubing 1918 can also include a lining that fits around the flexible tubing 1418. In some embodiments, the lining can prevent air or other fluids to seep between the flexible tubing 1918. The flexible tubing 1918 can be coupled to the outer sheath 1912. In some implementations, the flexible tubing 1918 can be surrounded by a sheath or lining to avoid frictional contact between the outer surface of the steerable tubing 1902 and other surfaces. In some implementations, the steerable instrument 1900 can be coated with Polytetrafluoroethylene (“PFTE”) to reduce frictional contact between the outer surface of the steerable instrument 1900 and other surfaces, such as the inner wall of the subject.

The flexible tubing 1918 can include or be coupled to one or more sensors, such as a light sensor, electromagnetic sensor, an optical stereotactic sensor, a pressure sensor, an impact sensor, a flow sensor, a radar sensor, a position sensor, or a distance sensor. In some embodiments, the flexible tubing 1918 detects a presence of the materials. The flexible tubing 1918 can be equipped with at least one sensor that can communicate with at least one external device, such as a sensor processing component (not shown) to determine the thickness of material relative the rest of the subject indicated by the sensor. The sensor can include, for example, a temperature sensor, a pressure sensor, a resistance sensor, an impact sensor, an ultrasonic sensor, or other sensor for medical examination. In some embodiments, the type of material is associated with at least an impedance or a density of the tissue. The sensor can gather temperature information and other sensed information, and provide signals corresponding to such information to the sensor-processing unit. The sensor-processing unit can subsequently identify the type of material. In some embodiments, the sensor can be an electrical sensor.

Referring now to FIG. 19A in conjunction with FIG. 19D, the steerable tubing 1902 can include a steerable tubing diameter 1920, and the flexible tubing 1918 can include a flexible tubing diameter 1922. The steerable tubing diameter 1920 can be less than 4.0 mm, such as for example, 3.9 mm. The steerable tubing diameter 1920 may be 3, 4, 5, 6, 7, 8, 9, or 10 mm. The flexible tubing diameter 1922 can be less than the steerable tubing diameter 1920 such that the flexible tubing 1918 can fit within the steerable tubing 1902. For example, the flexible tubing diameter 1922 can be 3.0 mm. The flexible tubing diameter 1922 can also be 3.9, 3.8, 3.7, 3.6, 3.5, 3.4, 3.3, 3.2, 3.1, 3.0, 2.9, 2.8, 2.7, 2.6, or 2.5 mm. In some embodiments, the flexible tubing diameter 1922 can be sized such that the flexible tubing 1918 fits within the steerable tubing 1902 without any gaps between the outer wall of the flexible tubing 1918 and the inner wall of the steerable tubing 1902. The fit can enable the outer wall of the flexible tubing 1918 to structurally support the steerable tubing 1902. In some embodiments, the flexible tubing 1918 has a 0.01, 0.02, 0.03, 0.04, 0.05, 0.06, 0.07, 0.08, 0.09, or 0.1 mm gap between the outer wall of the flexible tubing 1918 and the inner wall of the steerable tubing 1902.

Referring back to FIG. 19A in conjunction with FIG. 19C, the steerable instrument 1900 can include the first connector 1924 for articulating the distal end 1906 of the steerable tubing 1902 along the longitudinal axis 1926 extending through the steerable instrument 1900 responsive to receiving a first control input at the first connector 1924. The first connector 1924 can be coupled to the proximal end 1904 of the steerable tubing 1902. The first connector 1924 can be a knob, tube, handle, grip, or any other surface configured to receive control inputs from the operator. The control inputs can cause the first connector 1924 to bend the proximal end 1904 of the steerable tubing 1902 relative to the longitudinal axis 1926. For example, the control inputs on bend the proximal end 1904 of the steerable tubing 1902 by −90, −80, −70, −60, −50, −40, −30, −20, −10, 10, 20, 30, 40, 50, 60, 70, 80, or 90 degrees relative to the longitudinal axis 1926. By bending the steerable tubing 1902, the first connector 1924 can also bend the flexible tubing 1918 disposed within the steerable tubing 1902.

The first connector 1924 can be configured to articulate the distal end 1906 on the articulation axis 1928 relative to the longitudinal axis 1926. The articulation axis 1928 can be relative to the longitudinal axis 1926. For example, the articulation axis 1928 can be −90, −80, −70, −60, −50, −40, −30, −20, −10, 10, 20, 30, 40, 50, 60, 70, 80, or 90 degrees relative to the longitudinal axis 1926. The distal end 1906 can bend at an angle proportional to the angle bent by the proximal end 1904. For example, the steerable tubing 1902 can be configured to bend the distal end 1906 by 10 degrees responsive to a 10 degree bend of the proximal end 1904 by the first connector 1924. The steerable tubing 1902 also bends the flexible tubing 1918 disposed within. In another example, the steerable tubing 1902 is configured to bend according to any other configuration. For example, the steerable tubing 1902 can be configured to bend the distal end 1906 by 10 degrees responsive to a 10 degree bend of the proximal end 1904 by the first connector 1924, and bend the distal end 1906 by 15 degrees responsive to a 20 degree bend of the proximal end 1904 by the first connector 1924. In another example, the flexible tubing 1918 can be configured to bend the distal end 1906 by 10 degrees responsive to a 10 degree bend of the proximal end 1904 by the first connector 1924, and bend the distal end 1906 by 20 degrees responsive to a 20 degree bend of the proximal end 1904 by the first connector 1924.

In some embodiments, the first connector 1924 can be configured to input the τ-proximal used to rotate the flexible tubing 1918 and thus the outer sheath 1912 (or to apply a control torque corresponding to a desired τ-proximal, such as if the first connector 1924 includes gears and/or a motorized actuator to drive the rotation of the flexible tubing 1918). In some embodiments, the first connector 1924 is coupled to a motor configured to apply the torque or the control inputs. The distal end 1906 can rotate with an equivalent torque as the torque provided via the first connector 1924 at the proximal end 1904. It should be appreciated that using the distal end 1906 can be configured to rotate a specific degrees, equivalent or matching the degree of rotation of the proximal end 1904. Accordingly, the first contester 1924 can be configured to provide precision and control of the flexible tubing 1918 and thus the outer sheath 1912. For example, the operator may initiate a 30-degrees rotation of the first connector 1924. The rotation, force, and torque can be exerted to the distal end 1906 such that the outer sheath 1912 also rotates by 30-degrees.

The steerable instrument 1900 can include the flexible torque component 1930 disposed within the flexible tubing 1918. The flexible torque component 1930 can be coupled to and disposed within the inner sheath 1914. In addition, at least one of the elastomer or the friction reducing additive can reduce friction generated between the flexible torque component 1930 and the inner sheath 1914 when the flexible torque component 1930 and the inner sheath 1914 come in contact with one another, for example, when the steerable instrument 1900 has been passed through a tortuous pathway. The flexible torque component 1930 can be configured to rotate the inner sheath 1914 relative to the outer sheath 1912 to cut the material. The flexible torque component 1930 can be composed of at least one of metal, steel, plastic, titanium, nickel, carbon fiber, or other alloys. In some embodiments, the inner sheath 1914 can include a lining within which the flexible torque component 1930 is disposed.

The steerable instrument 1900 can include the second connector 1932 coupled to the proximal end 1904 of the steerable tubing 1902 and configured to rotate the flexible torque component 1930. The second connector 1932 can be coupled to the flexible torque component 1930. The second connector 1932 can be configured to receive control inputs flora the operator. The second connector 1932 can be a knob, tube, handle, grip, or any other surface configured to receive control inputs.

The control inputs can rotate the second connector 1932 to cause the inner sheath 1914 to rotate relative to the outer sheath 1912 to cut the material. The control inputs can rotate the second connector 1932 any number of degrees. For example, the control inputs can rotate the second connector 1932 by 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, or 360-degrees relative to the longitudinal axis 1926. The inner sheath 1914 can rotate any number of degrees. For example, the inner sheath 1914 can rotate 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, or 360-degrees. The inner sheath 1914 can rotate at an angle proportional to the angle of rotation of the second connector 1932. For example, the inner sheath 1914 can be configured to rotate by 10 degrees responsive to a 10 degree rotation of the second connector 1932. In and example, the inner sheath 1914 is configured to bend according to any other configuration. For example, the inner sheath 1914 can be configured to rotate by 10 degrees responsive to a 10 degree minion by the second connector 1932, but rotate by 15 degrees responsive to a 24 degree rotation by the second connector 1932. In another example, the inner sheath 1914 can be configured to rotate by 10 degrees responsive to a 10 degree rotation by the second connector 1932, but rotate by 25 degrees responsive to a 20 degree rotation by the second connector 1932.

The flexible tubing 1918 can include an aspiration channel 1934 extending from the cutting window 1916 to the aspiration port 1936. The aspiration channel 1934 can be partially defined by the flexible torque component 1930. The aspiration channel 1934 can be partially defined by an outer wall of the inner sheath 1914. The aspiration channel 1934 can be partially defined by an inner wall of the outer sheath 1912. Materials can enter the aspiration channel 1934 via the rutting window 1916 and traverse the length of the aspiration channel 1934 to the aspiration port 1936.

The aspiration port 1936 can be an opening or any other connection between the flexible tubing 1918 and a vacuum source 1938. The aspiration port 1936 can include sockets, plugs, or any other coupling mechanism configured to couple the flexible tubing 1918 and a vacuum source 1938. In some embodiments, the aspiration port 1916 can include additional tubing or hosing to couple the vacuum source 1938 to the flexible tubing 1918.

The vacuum source 1938 can retrieve, extract, or collect cut material from the aspiration channel 1934. The vacuum source 1938 can be configured to pull, draw, or drag the material. The vacuum source 1938 can be configured to initiate a suction feature or force to retrieve cut materials. The vacuum source 1938 can be configured to suction liquid, fluid, or gas from the aspiration channel 1934. The aspiration channel 1934 can be configured to enable the vacuum source 1938 to maintain a suction force throughout the length of the aspiration channel 1934 by preventing air from escaping or entering through the aspiration channel 1934. The vacuum source 1938 can apply a vacuum pressure greater than or equal to 200 mmHg and less than or equal to 750 mmHg to retrieve the cut materials through the aspiration channel 1934. Accordingly, the vacuum source 1938 can be configured to aspirate suction, or pull materials into aspiration channel 1934 for retrieval or extraction of the material. In some embodiments, vacuum source 1938 can include a collection midge or a repository for storing the cut materials or any other substance retrieved from the subject using the vacuum source 1938.

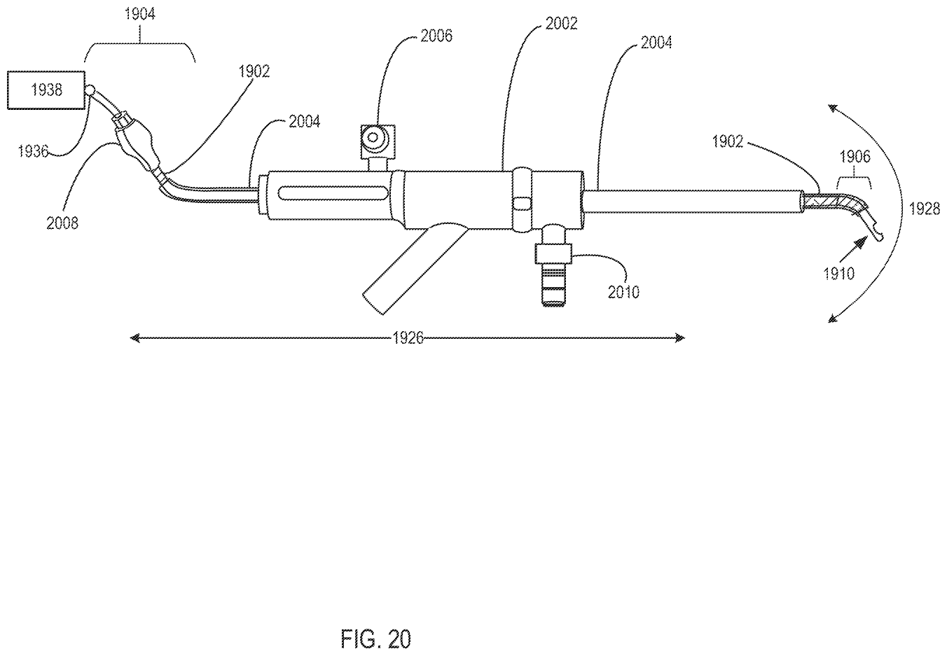

Referring to FIG. 20, shown is a view of a surgical instrument 2002 for maneuvering the steerable instrument 1900 to a treatment site during a laparoscopic or hysteroscopic procedure according to embodiments of the present disclosure. The surgical instrument 2002 can be inserted, situated, or resided in the subject. The surgical instrument 2002 can be inserted into an opening or a cavity, such as those shown in FIGS. 1A-1D. The insertion of the surgical instrument 2002 can be through the opening or the cavity of the subject. The surgical instrument 2002 can be a flexible hysteroscope or laparoscope, such that the surgical instrument 2002 can be turned, bent, or otherwise navigated through curvatures of the subject.

While it is difficult to utilize the surgical instrument 1900 with the surgical tool 2002 in cavities and other narrow or tortuous treatment sites, the flexibility and small diameter of the surgical instrument 1900 can address this problem by enabling the surgical instrument 1900 to be inserted into the surgical tool 2002. The instrument 1900 and the surgical tool 2002 can be navigated together to the treatment site, where the sable instrument 1900 can articulate its distal end 1906 to maneuver the cutting assembly 1902 to the material. For example, the surgical instrument 1900 can cut the material while the surgical tool 2002 provides a camera or irrigation fluid.

The surgical instrument 2002 can include tubing 2004 that defines a working channel or instrument channel. The steerable instrument 1900 can be inserted into the surgical instrument 2002 through the tubing 2004. The length of the steerable instrument 1900 can be sized to exceed the length of the surgical instrument 2002 or the tubing 2004. For example, the length of the steerable instrument 1900 can be 100, 200, 350, 300, 730, or 900 mm longer than the length of the surgical instrument 2002 or the tubing 2004. The steerable instrument 1900 can extend any distance past a distal end of the surgical instrument 2002 or the tubing 2004. For example, the steerable instrument 1900 can extend 10, 20, 30, 40, 30, 60, 70, 80, 90, or 100 mm past the distal end of the surgical tool. The steerable instrument 1900 can be sized, shaped or configured such that the steerable tubing diameter 1920 is less than the diameter of the channel in which the steerable instrument 1900 is to be inserted. For example, the steerable instrument 1900 can be sized such that the steerable tubing diameter 1920 is 0.1, 0.2, 0.3, 0.4, 0.5, or 1 mm less than the diameter of the channel of the surgical instrument 2002.

The surgical instrument 2002 can include an irrigation entry port 2006. The irrigates entry port 2006 can be configured to introduce irrigation fluid into the surgical inurement 2002. The irrigation entry port 2006 can be configured to engage with an irrigation source, such as a saline or water container. In some implementations, the irrigation entry port 2006 can be a Y port used in fluid delivery systems that complies with medical device industry standards. The surgical instrument 2002 can be configured such that the irrigation fluid flows between the outer wall of the steerable instrument 1900 and the inner wall of the channel within the surgical instrument 2003. The irrigation fluid can then be released at a distal end of the surgical instrument 2002.

The steerable instrument 1900 can include controller 2008 coupled to the steerable instrument 1900. The controller 2008 can be an embodiment and/or perform the functionality of the first connector 1924 and/or the second connector 1932. In some embodiments, the controller 2008 can be configured to receive a pushing or pulling force to maneuver the steerable instrument 1900 into the tubing 2004. In some embodiments, the pushing and pulling can cause the steerable instrument 1900 to expand or retract relative to the tubing 2004. In some embodiments, the controller 2008 can be configured to receive a pushing or pulling force to provide axial movement of the distal end 1906. The flexible tubing 1918 can be configured to receive axial movement at a proximal end 1904 such that there is axial movement at the distal end. In some embodiments, the controller 2008 can be configured to receive control inputs to maneuver the proximal end 1904 sigh that the distal end 1906 of the flexible tubing 1918 can maneuver along the articulation axis 1928. For example, the controller 2008 can be configured such that a 60-degree bend relative to the longitudinal axis 1926 causes a 60-degree bend of the distal end 1906. In some embodiments, the controller 2008 can be configured to receiver control inputs to rotate the flexible torque component 1930 to rotate the inner sheath 1914 to cut materials by the cutting assembly 1914. For example, the controller 2008 can be configured such that a 60-degree rotation causes the flexible torque component 1930 and the inner sheath 1914 to rotate by 60-degrees to cut the material. In some embodiments, the controller 2008 can be configured to receive torque to rotate the proximal end 1904 of the flexible tubing 1918 and the outer sheath 1912. For example, the steerable instrument 1900 can be configured such that a 360-degree rotation of the controller 2008 causes a 360-degree rotation of the proximal end 1904 and the distal end 1906 of the flexible tubing 1918, and thus the outer sheath 1912.

The surgical instrument 2002 can include a light 2010 configured to illuminate the treatment site. The light 2010 can be a fiber optic light, a light emitting diode (“LED”), incandescent lamps, compact fluorescent, halogen, neon, or other types of lighting elements. In some embodiments, the contract 2008 can actuate the light 2010 to turn it on, off, or modulate its intensity.

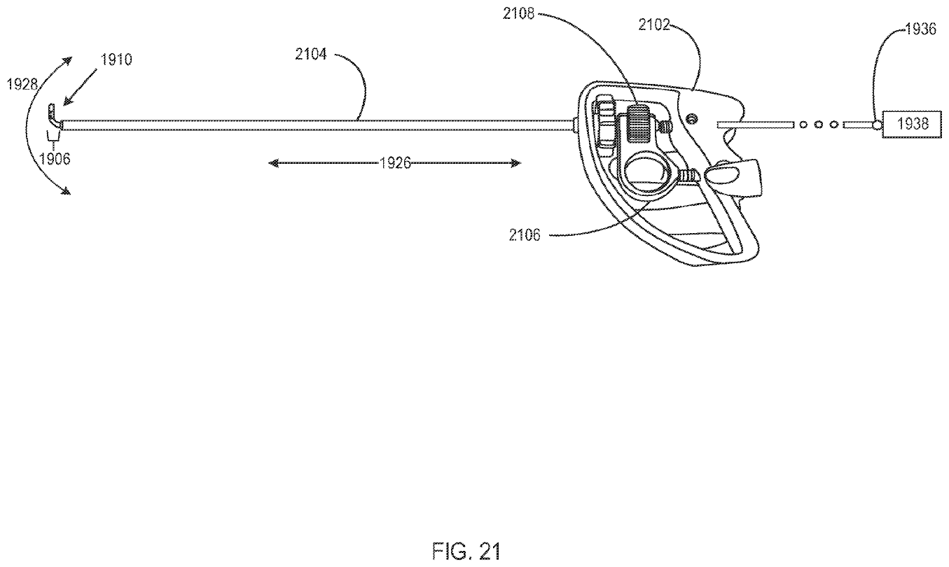

Referring to FIG. 21, shown is a view of a surgical tool 2102 for maneuvering the steerable, instrument 1900 to a treatment site during a laparoscopic or hysteroscopic procedure according to embodiments of the present disclosure. The surgical tool 2102 can be inserted, situated or resided in the subject. The surgical tool 2102 can be inserted into an opening or a cavity, such as those shown in FIGS. 1A-1D. The insertion of the surgical tool 2102 can be through the opening or the cavity of the subject. The surgical tool 2102 can be a flexible hysteroscopic or laparoscope, such that the surgical tool 2102 can turn, bend, or otherwise navigate through curvature of the subject.

While it is difficult to utilize the surgical instrument 1900 with the surgical tool 2102 in cavities and other narrow or tortuous treatment sites, the flexibility and small diameter of the surgical instrument 1900 can address this problem by enabling the surgical instrument 1900 to be inserted into the surgical tool 2102. The instrument 1900 and the surgical tool 2102 can be navigated together to the treatment site where the steerable instrument 1900 can articulate its distal end 1906 to maneuver the cutting assembly 1902 to the material. For example, the surgical instrument 1900 tan cut the material while the surgical tool 2102 provides a camera or irrigation fluid.

The surgical tool 2102 can include tubing 2104 that defines a working channel or instrument channel. The steerable instrument 1900 can be inserted into a surgical tool 2102 through the tubing 2104. The length of the steerable instrument 1900 can be sized to exceed the length of the surgical tool 2102 or the tubing 2104. For example, the length of the steerable instrument 1900 can be 100, 200, 350, 500, 750, or 900 mm longer than the length of the surgical tool 2102 or the tubing 2104. The steerable instrument 1900 tan extend any distance past a distal end of the surgical tool 2102. For example, the steerable instrument 1900 can extend 10, 20, 30, 40, 50, 60, 70, 80, 90, or 100 mm past the distal lend of the surgical tool.

The surgical tool 2102 can include a first connector 2106. The first connector 2106 can be coupled to the steerable tubing 1902. The first connector 2106 can be a lever, a trigger, or any other mechanism configured to receive control inputs from the operator. The first connector 2106 can be an embodiment and/or perform the functionality of the first connector 1924. The first connector 2106 and the second connector 2108 can be coupled to the steerable tubing 1902. In some embodiments, the first connector 2106 can be configured to receive a pushing or pulling force from the operator, and provide the pushing or pulling force to the steerable instrument 1900. In some embodiments, the pushing or pulling force provided by the first connector 2106 can provide axial movement of the distal end 1906. For example, the pushing or pulling force provided by the first connector 1924 can maneuver the distal end 1906 of the steerable tubing 1902 along the articulation axis 1928. For example, the first connector 2106 can be configured such that a first force cause a 30-degree bend of the distal end 1906, and a second force causes a 60-degree bend of the distal end 1906. The second force can be stronger than the first force. For example, the first force can be 5 N, and the second force can be 10 N.

The surgical tool 2102 can include a second connector 2108. The second connector 2108 can be coupled to the steerable tubing 1902. The first connector 2106 can be a wheel, a knob, or any other mechanism configured to rive control inputs from the operator. The second connector 2108 can be an embodiment and/or perform the functionality of the second connector 1932. In some embodiments, the second connector 2108 can be configured to receive control inputs to rotate the flexible torque component 1930 rotate the inner sheath 1914 to cut materials by the cutting assembly 1910. For example, the second connector 2108 can be configured such that a 60-degree rotation causes the flexible torque component 1930 and the inner sheath 1914 to rotate by 60-degrees to cut the material. In some embodiments, the second connector 2108 can be configured to receive torque to rotate the proximal end 1904 of the steerable tubing 1902 such that the flexible tubing 1918 rotates, which causes the outer sheath 1912 to rotate. For example, the second connector 2108 can be configured such that a 360-degree rotation causes a 360-degree rotation of the proximal end 1904 and the distal end 1906 of the steerable tubing 1902, which rotates the flexible tubing 1918, and thus the outer sheath 1912.

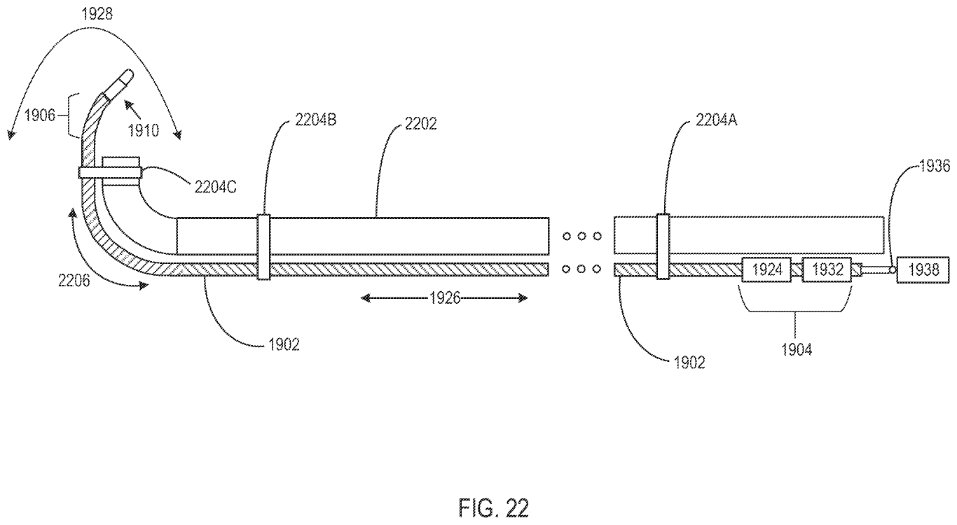

Referring to FIG. 22, shown is a view of a surgical tool 2202 for maneuvering the steerable instrument 1900 to a treatment site during a laparoscopic of hysteroscopic procedure according to embodiments of the present disclosure. The surgical tool 2202 can be inserted, situated, or resided in the subject. The surgical tool 2202 can be inserted into an opening or a cavity, such as those shown in FIGS. 1A-1D. The insertion of the surgical tool 2202 can be through the opening or the cavity of the subject. The surgical tool 2202 can be a flexible hysteroscope or laparoscope, such that the surgical tool 2202 can turn, bend, or otherwise navigate through curvature of the subject.

The surgical tool 2202 and the steerable instrument 1900 can be coupled by one or more attachment members 2204a-2204n (generally referred to as attachment members 2204). While it is difficult to utilize the surgical instrument 1900 with the surgical tool 1202 in cavities and other narrow or tortuous treatment sites, the attachment members 2204 can address this problem by enabling the surgical instrument 1900 to attach and navigate along an external side of the surgical tool 2202. The attachment members 2204 enable the surgical instrument 1900 and the surgical tool 2202 to be navigated together to the treatment site, where the steerable instrument 1900 can articulate its distal end 1914 to maneuver the cutting assembly 1910 to the material. For example, the instrument 1900 can cut the material while the surgical tool 2202 provides a camera, irrigation, or suction. In another example, the steerable instrument 1900 does not have or use the aspiration channel 1936 if the steerable instrument 1900 is utilized with the surgical tool 2202.

The attachment members 2204 can attach to the surgical tool 2202. The attachment members 2204 can be configured along the lengths of the flexible tubing 1918 and the surgical tool 2200. The attachment members 2204 can include bands or loops (e.g., fishing pole loops). Each of the attachment members 2204 can have an opening configured to receive the steerable instrument 1900. In some embodiments, the opening can be sized such that the steerable instrument 1900 snugly fits within the opening without any gaps between the outer wall of the steerable instrument 1900 and the inner wall of the opening. A diameter of the opening diameter can be less than 4 mm. The diameter can be 5 mm. The diameter may be 5.8 mm. The diameter can be 10 mm. The diameter ram be less than 0.5 inches. The diameter can be less than 0.25 inches. The diameter may be greater than or equal to 0.03 inches and less than or equal to 0.3 inches. The diameter may be greater than or equal to 0.11 inches and less than or equal to 0.13 inches.

The lend of the steerable instrument ent 1900 can be sized to exceed the length of the surgical tool 2202. For example, the length of the steerable instrument 1900 can be 100, 200, 350, 500, 750, or 900 mm longer than the length of the surd tool 2202. The surgical tool 2202 can have an outside diameter of 5.8 mm, an inside diameter of 4.5 mm, and a length of 1200 mm. The steerable instrument 1900 can extend any distance past a distal end of the surgical tool 2202. For example, the stowable instrument 1900 can extend 10, 20, 30, 40, 50, 60, 70, 80, 90, or 100 mm past the distal end of the surgical tool.

The attachment members 2204 can include one or more substances, such as rubber, cloth, metal, steel, plastic, titanium, nickel, carbon fiber, or other alloys. The attachment members 2204 can include one or more textures or grooves, such as a spiral a twist, frets, or other protrusion or engraving. The attachment members 2204 may be coated with at least one chemical compound for insertion into the subject, such as polymer, hydrophilic, nitinol, fluoropolymer, or a combination of two or more compounds to increase durability, lubrication, flexibility, or corrosion resistance of the attachment members 2204. The attachment members 2204 can be flexible as to not introduce injuries, tears, wounds, or other damages within the subject, to the flexible tubing 1918, or to the surgical tool 2202.

The steerable instrument 1900 can pass through and out of the attachment members 2204. For example, the proximal end 1904 of the steerable tubing 1902 can be configured to receive the pushing or pulling force from the operator, and provide the pushing or pulling force to the distal end 1906. The steerable tubing 1902 can rotate within the attachment members 2204. For example, the proximal end 1904 of steerable tubing 1902 can be configured to receive control inputs to rotate the steerable tubing 1902 and thus the flexible tubing 1918 to rotate the outer sheath 1912. For example, a 60-degree rotation of the proximal end 1904 can cause a 60-degree rotation of the distal end 1906.

The surgical tool 2202 can include a bend 2206. For example, the bend 2206 can be a section of the surgical tool 2202 that is bent or is shred to the treatment site. The bend 2206 can define an angle. For example, the angle can be −90, −80, −70, −60, −50, −40, −30, −20, −10, 10, 20, 30, 40, 50, 60, 70, 110, or 90 degrees relative to the longitudinal axis 1926. The bent portion of the surgical tool 2202 can define a radius. The radius can be 1, 2, 3, 4, 5, 6, 7, 8, 9, or 10 mm. The steerable instilment 1900 can maneuver along the bend 2206 through each of the attachment members 2204. When the steerable instrument 1900 is secured to the surgical tool 2202 via the attachment members 2204, the first connector 1924 can articulate the distal end 1906 or rotate the outer sheath 1912, and the second connector 1932 can rotate the inner sheath 1914. The first connector 1924 and the second connector 1932 can articulate the distal end 1906 and rotate the outer sheath 1912 and the inner sheath 1914 regardless of the angle formed by the bend 2206.

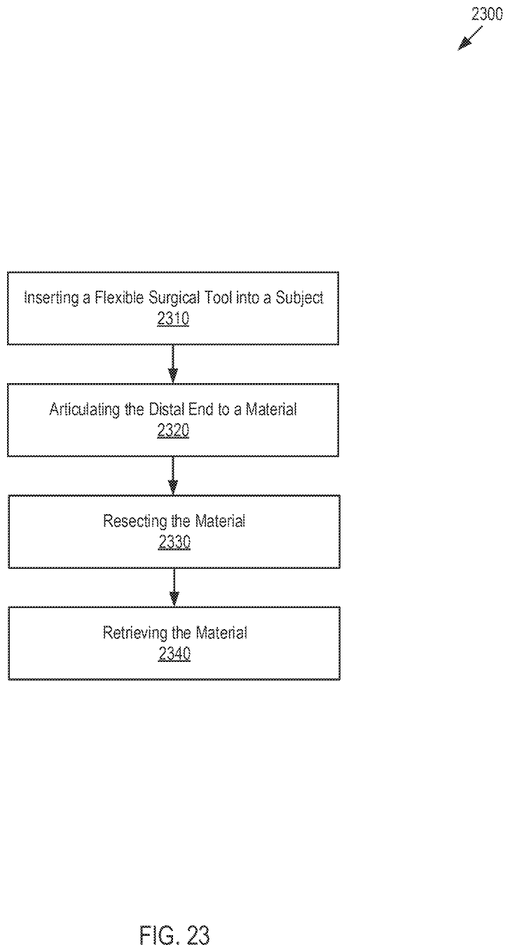

Referring to FIG. 23, a method 2300 of performing a laparascopic or hysteroscopic procedure using the surgical instrument can be shown. The method 2300 can be performed using various embodiments of surgical instruments described herein. The method 2300 or steps thereof can be repeated, such as to address multiple treatment sites having multiple materials to be cut, both inside and outside the vessels.

At 2310, a steerable instruments (e.g., steerable instrument 1900) can be inserted into a subject. The steerable instruments can be disposed within a working channel of a surgical tool (e.g., surgical tool 2002 or surgical tool 2102). The steerable instrument can include a steerable tubing (e.g., steerable tubing 1902). The steerable tubing can include a surgical instrument surgical instrument 1908). The steerable instrument can include a cutting assembly (e.g., cutting assembly 1910) configured to cut the material. The cutting assembly can include an outer sheath (e.g., outer sheath 1912) and an inner sheath (e.g., inner sheath 1914) disposed within the outer sheath, the outer sheath defining a cutting window (e.g., cutting window 1916). The cutting assembly can be coupled to a distal end of a flexible tubing (e.g., flexible tubing 1918).

The cavity can be a body cavity or a spacing inside the body, such as the uterus, fallopian tubes, ovaries, mouth, the ear, the nose, the esophagus, etc. The cavity may be generated using at last one surgical procedure, such as a cut, a drill, or a dissection. The generated cavity can be in various different portions of the body of the subject, such as the uterus, arm, the stomach, the liver, the neck, etc.

The treatment site can include material to be cut from the subject. The material can include foreign material introduced into the subject, a solidified material clogging the passage of a vessel, or other material determined to be cut the subject. The surgical tool may include one or more sensors, light source, or other attachments to facilitate the movement or navigation of the surgical tool towards the treatment site. The attachments can facilitate identification of the material at the treatment site, such as to receive visual feedback to indicate the material at the treatment site. The one or more sensors can include, for example, a tilt sensor, a proximity sensor, a light sensor, a pressure sensor, a flow sensor, an impact sensor, an ultrasound sensor, a distance sensor, or other sensors to facilitate an endoscopic procedure or operation. For example, a doctor or an operator may determine a location of the material using a non-intrusive imaging techniques, such as an x-ray, an ultrasound, or a computer tomography (“CT”) scan. In addition, the material can be located by navigating the surgical tool to the treatment site and using the one or more sensors to identify the material, such as the camera or the light source. The surgical tool may reach the treatment she or the material based on, for example, sensing a blockage within the vessel of the subject using the one or more sensor. The material may be identified using the camera of the surgical tool and display an image on a display device external to the surgical tool.

The procedure can include inserting a surgical tool into the cavity of the subject. For example, a treatment site can be determined within a vessel of a subject, such as an artery, an arteriole, a capillary, a venule, or a vein. A cavity can be identified to lead to the vessel containing the treatment site. A doctor (or an operator) can insert the surgical tool into the cavity leading to the vessel. The doctor can navigate the surgical tool to the treatment site of the vessel. The surgical tool can stop or terminate the navigation of the surgical tool in response to reaching the treatment site. In some cases, the reached treatment site can be based on a camera inserted with or as a part of the surgical tool. In some cases, the reached treatment site can be based on a length of the inserted surgical tool. The length of the inserted surgical tool can be determined based on a predetermined location of the treatment site via using x-ray, computer tomography (“CT”) scan, ultrasound, magnetic resonance imaging (“MRI”), or other non-intrusive imaging techniques.

In some embodiments, the surgical tool may be inserted into the subject in conjunction with the surgical tool. In conjunction may refer to together with, at the same time as, or in an instance with, for example, the surgical tool. The operator or the doctor may insert the surgical tool, enclosing the surgical tool, into the subject via the cavity. The surgical tool may extend from the surgical tool, such as through a hollow portion of the surgical tool, and move deeper into the subject. The surgical toot can move along the surgical tool to proceed deeper into the subject. For example, at this point, the surgical tool can be fixed in a location, a distance away from a distal end of the surgical tool, within the subject. The process may be repeated for the surgical tool to reach or pass a treatment site to perform other laparoscopic or hysteroscopic procedures.

The surgical tool may use the at least one sensor to navigate within the subject, determine a location of the material within the subject, and initiate the rotation of the cutting assembly in response to positioning the distal tube end of the surgical tool at or near the treatment site. The positioning of the surgical tool can refer to, for example, in contact with, 0.1 millimeter, 0.5 millimeter, 1 millimeter, or 1.5 millimeters from the material (or the treatment site).

The surgical tool may be used to navigate or guide the material cutting device within the body of the subject along any tortuous path. Accordingly, the surgical tool may determine the positioning of the surgical tool to initiate a rotation for the cutting assembly to perform a cut, an extraction, or a debriding of a material within the subject. The determination to initiate the rotation can be based on, for example, a coupling or engagement between the cutting assembly and the surgical tool.

The surgical tool can pass the treatment site, such as through material located at the treatment site. The navigation or driving of the surgical tool can be terminated in response to reaching, being in contact with, or passing the material or the treatment site. The laparoscopic or hysteroscopic procedure can include determining the location of a material or a treatment site. In some implementations, an operator may use at least one imaging tool to identify the location of the material, such as an x-ray, an MRI, or a CT scan. In some implementations, the operator or the doctor can utilize a camera or a scope to locate the material within the subject. The camera or the scope can be a pan of the surgical tool. For example, the operator may insert the surgical tool twice to perform the material removal operation or procedure. Once for identifying the material, and the second to collect, extract, debride, or cut the material. In another example, the operator may insert the surgical tool into the subject. The operator can navigate the surgical tool within the subject to find the material. Once the material is found, the operator may initiate a rotation to the cutting assembly to debride and cut the cut materials. The process of debriding the material may be referred to as removing the material. In this example, the material cutting device may be inserted once to complete the laparoscopic or hysteroscopic operation or procedure.

The extension of the surgical tool can move towards a treatment site within the subject. The surgical tool may extend or move pass the treatment site, in which an operator can terminate further extension of the surgical tool into the subject. The surgical tool can move towards the treatment site using the surgical tool. While moving towards the treatment site, the operator may push or exert a force to the proximal end of the surgical tool. The surgical tool can be moved further inside the subject and towards the treatment site in response to the force exerted to the proximal end.

In some embodiments, the surgical tool can provide or transmit at least one substance to a treatment site of a subject. The substance can include liquid, gas, or other chemical compounds. The substance may facilitate the process of debriding the material into the cut materials, for example, by releasing a gaseous substance to soften, disperse, or breakdown the material. Accordingly, the provision of the substance can assist the debriding process using the cutting assembly. In another example, the substance may assist in healing the subject, for example, by blocking a damaged portion of the vessel or by providing a medication to the treatment area within the vessel.

At 2320, the distal end (e.g., distal end 1906) of the steerable tubing can be articulated. A first control input can be applied to a first connector (e.g., first connector 1924) coupled to the proximal end of the steerable tubing to cause articulation of the distal end of the steerable tubing along a longitudinal axis extending through the surgical tool. For example, the control inputs can bend the proximal end of the flexible outer tubing by −90, −80, −70, −60, −50, −40, −30, −20, −10, 10, 20, 30, 40, 50, 60, 70, 80, or 90 degrees relative to the longitudinal axis. The first control input can cause the distal end to twist and turn in various angles to maneuver the cutting assembly to the material.

The first connector can articulate the distal end on the articulation axis relative to the longitudinal axis. The articulation axis can be relative to the longitudinal axis. For example, the articulation axis can be −90, −80, −70, −60, −50, −40, −30, −20, −10, 10, 20, 30, 40, 50, 60, 70, 80, or 90 dew relative to the longitudinal axis. The distal end can bend at an angle proportional to the angle bent by the proximal end. For example, the steerable tubing can bend the distal end by 10 degrees responsive to a 10-degree bend of the proximal end by the first connector. In another example, the steerable tubing can bend according to any other configuration. For example, the steerable tubing can bend the distal end by 10 degrees responsive to a 10-degree bend of the proximal end by the first connector, and bend the distal end by 15 degrees responsive to a 20-degree bend of the proximal end by the first connector. In another example, the steerable tubing can bend the distal end by 10 degrees responsive to a 10-degree bend of the proximal end by the first connector, and bend the distal end by 25 degrees responsive to a 20-degree bend of the proximal end by the first connector. The distal end of the steerable tubing may be positioned a distance from the material, such as 1 millimeter, 1 inch, or 1 meter from the material.

In some embodiments, the control input can provide torque at the proximal end of the steerable tubing to the outer sheath. For example, the control inputs can rotate the proximal end by 60-degrees to cause 60-degree 60-degree rotation of the distal end. In some embodiments, the first connector can input the τ-proximal used to rotate the steerable tubing and thus the outer sheath (or to apply a control torque corresponding to a desired τ-proximal, such as if the first connector includes gears and/or a motorized actuator to drive the rotation of the suable tubing). In some embodiments, the first connector receives the torque or the control inputs from a motor. The distal end can rotate with an equivalent torque as the torque provided via the first connector at the proximal end. It should be appreciated that the distal end can be configured to rotate a specific degree, equivalent or matching the degree of rotation of the proximal end. Accordingly, the first connector can provide precision and control of the steerable tubing and thus the outer sheath. For example, the operator may initiate a 30-degrees rotation of the first connector. The rotator, force, and torque can be exerted to the distal end such that the outer sheath also rotates by 30-degrees.

At 2330, the coning assembly can cut the material from the treatment site. A second control input can be applied to a second connector (e.g., second connector 1932) coupled to the proximal end of the steerable tubing to rotate a flexible torque component (e.g., flexible torque component 1930) disposed within the flexible tubing. In some cases, an operator may exert a manual or mechanism rotation as the second control input applied to the second connector.

The flexible torque component can be coupled to the inner sheath. The inner sheath can be rotated in response to receiving an excited rotational force or torque from the second connector. The flexible torque component can cause the inner sheath to rotate relative to the outer sheath to cut the material. These removal of the material can refer to cutting, debriding, pulling, dissecting, or tearing the material from the treatment site. The cutting assembly can cut the material in response to the rotation by the inner sheath. The torque can be provided by the second connector. The exerted rotation may traverse from the proximal end to the distal end of the steerable tubing. As an example, the second connector may provide 180 degrees rotation to the steerable tubing at a proximal end, and the distal end will rotate by 180 degrees.

At 2340, the material can be retrieved by using the surgical tool. The substances can include the cut materials, liquid, gas, or other chemical compounds within the body of the subject. The operator can actuate a vacuum source (e.g., vacuum source 1938) coupled to the flexible surgical too to provide suction through an aspiration channel (e.g., aspiration channel 1934) defined by an inner wall of the flexible tubing to cut the material from the subject via the aspiration channel. The process of retrieving the cut materials may be concurrent to the debriding process by the cutting assembly. For example, the vacuum source can initiate a vacuum to withdraw the cut materials while the cutting assembly d brides the material. The cut materials can be stored in a container or a repository in the vacuum source and/or external to the flexible surgical too.

In further example, responsive to the cutting assembly debriding a material into cut materials, the vacuum source may pull in, withdraw, or pump out the cut materials from the subject. The cut materials can be withdrawn via an aspirations channel. The process of withdrawing the cut material can be performed by a vacuum source. The vacuum source can be external to the surgical tool. The vacuum source device can be initiated by a signal or a mechanical trigger. A pump device may be connected to the aspiration channel configured to withdraw of the cut materials from the subject. The pump device may pull a substance from a repository and push the chemical into the subject. The pump device may withdraw the cut materials into a second repository for storage.

The surgical tool may be cut from the subject upon or based on completion of the laparoscopic or hysteroscopic procedures or processes. The completion of the laparoscopic or hysteroscopic procedures can entail the debriding of the material, such as through an entire treatment site, or the collection of the debriding materials. For example, the treatment site can include a loth of 3 inches. The surgical tool may initiate a rotation of the cutting ably and navel through the 3 inches of the treatment site to debride the material. While debriding the material, the surgical tool may retrieve or draw in the cut materials into the aspiration channel. For example, once the surgical tool cuts the material through the 3 inch length of the treatment site and retrieve the cut materials, the laparoscopic or hysteroscopic procedures may be completed.

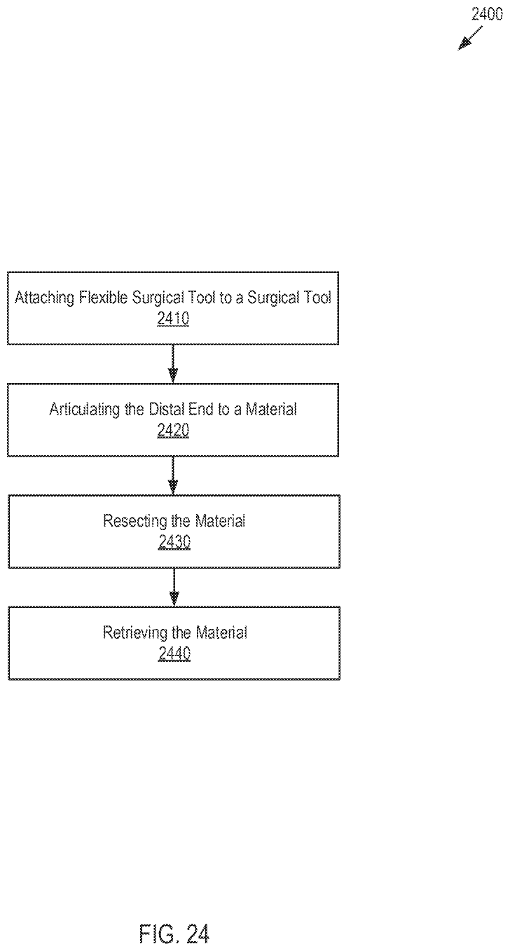

Referring to FIG. 24, a method 2400 of performing a laparascopic or hysteroscopic procedure using the surgical tool can be shown. The method 2400 can be performed using various embodiments of surgical tools described herein. The method 2400 or steps thereof can be repeated such as to address multiple treatment sites having multiple materials to be cut, both inside and outside the vessels.

At 2410, a steerable instrument (e.g., steerable instrument 1900) can be attached to a surgical tool (e.g., surgical tool 2202). The steerable instrument can include a steerable tubing (e.g., steerable tubing 1902). The steerable tubing can include a surgical instrument (e.g., surgical instrument 1908). The surgical instrument can include a cutting assembly (e.g., cutting assembly 1910) configured to cut the material. The cutting assembly can include an outer sheath (e.g., outer sheath 1912) and an inner sheath (e.g., inner sheath 1914) disposed within the outer sheath, the outer sheath defining a cutting window (e.g., cutting window 1916). The cutting assembly can be coupled to a distal end of a flexible tubing (e.g., flexible tubing 1918). The surgical tool and the surgical tool can be attached with attachment members (e.g., attachment members 2204). The attachment members can be positioned along the surgical tool. The surgical tool can be inserted into each attachment member. The magical tool can be maneuvered through each of the attachment members along the surgical tool to attach the surgical tool to the surgical tool. The surgical tool and the attached surgical tool can be inserted into a subject.

The cavity can be a body cavity or a spacing inside the body, such as the uterus, fallopian tubes, ovaries, mouth, the ear, the nose, the esophagus, etc. The cavity may be generated using at least one surgical procedure, such as a cut, a drill, or a dissection. The generated cavity can be in various different portions of the body of the subject, such as the uterus, arm, the stomach, the liver, the tuck, etc.

The treatment site can include material to be cut from the erect. The material can include foreign material introduced into the subject, a solidified material clogging the passage of a vessel, or other material detrained to be cut from the subject. The surgical tool may include one or more sensors, light source, or other attachments to facilitate the movement or navigation of the surgical tool towards the treatment site. The attachments can facilitate identification of the material at the treatment site, such as to receive visual feedback to indicate the material at the treatment site. The one or more sensors can include, for example, a tilt sensor, a proximity sensor, a light sensor, a pressure sensor, a flow sensory an impact sensor, an ultrasound sensor, a distance sensor, or other sensors to facilitate an endoscopic procedure or operation. For example, a doctor or an operator may determine a location of the material using a non-intrusive imaging techniques, such as an x-ray, an ultrasound, or a computer tomography “CT”) scan. In addition, the material can be located by navigating the surgical tool to the treatment site and using the one or more sensors to identify the material, such as the camera or the light source. The surgical tool may reach the treatment site or the material based on, for example, sensing a blockage within the vessel of the subject using the one or more sensor. The material may be identified using the camera of the surgical tool and display an image on a display device external to the surgical tool.

The procedure can include inserting a surgical tool into the cavity of the subject. For example, a treatment site can be determined within a vessel of a subject, such as an artery, an arteriole, a capillary, a venule, or a vein. A cavity can be identified to lead to the vessel containing the treatment site. A doctor (or an operator) can insert the surgical tool into the cavity leading to the vessel. The doctor can navigate the surgical tool to the treatment site of the vessel. The surgical tool can stop or terminate the navigation of the surgical tool in response to reaching the treatment site. In some cases, the reached treatment site can be based on a camera inserted with or as a part of the surgical tool. In some cases, the reached treatment site can be based on a length of the inserted surgical tool. The length of the inserted surgical tool can be determined based on a predetermined location of the treatment site via using x-ray, computer tomography (“CT”) scan, ultrasound, magnetic resonance imaging (“MRI”), or other non-intrusive imaging techniques.

In some embodiments, the surgical tool may be inserted into the subject in conjunction with the surgical tool. In conjunction may refer to together with, at the same time as, or in an instance with, for example, the surgical tool. The operator or the doctor may insert the surgical tool, enclosing the surgical tool, into the subject via the cavity. The surgical tool may extend from the surgical tool, such as through a hollow portion of the surgical tool, and move deeper into the subject. The surgical tool can move along the surgical tool to proceed deeper into the subject. For example, at this point, the surgical tool can be fixed in a location, a distance away from a distal end of the surgical tool, within she subject. The process may be repeated for the surgical tool to reach or pass a treatment site to perform other laparoscopic or hysteroscopic procedures.

The surgical tool may use the at least one sensor to navigate within the subject, determine a location of the material within the subject, and initiate the rotation of the cutting assembly in response to positioning the distal tube end of the surgical tool at or near the treatment site. The positioning of the surgical tool can refer to, for example, in contact with, 0.1 millimeter, 0.5 millimeter, 1 millimeter, or 1.5 millimeters from the material (or the treatment site).

The surgical tool may be used to navigate or guide the material cutting device within the body of the subject along any tortuous path. Accordingly, the surgical tool may determine the positioning of the surgical toot to initiate a rotation for the cutting assembly to perform a cut, an extraction, or a debriding of a material within the subject. The determination to initiate the rotation can be based on, for example, a coupling or engagement between the cutting assembly and the surgical tool.

The surgical tool can pass the treatment site, such as through material located at the treatment site. The navigation or driving of the surgical tool can be terminated in response to teaching, being in contact with, or passing the material or the treatment site. The laparoscopic or hysteroscopic procedure can include determining the location of a material or a treatment site. In some implementations, an operator may use at least one imaging tool to identify the location of the material, such as an x-ray, an MRI, or a CT scan. In some implementations, the operator or the doctor can utilize a camera or a scope to locate the material within the subject. The camera or the scope can be a part of the surgical tool. For example, the operator may insert the surgical tool twice to perform the material removal operation or procedure. Once for identifying the material, and the second to collect, extract, debride, or cut the material. In another example, the operator may insert the surgical tool into the subject. The operator can navigate the surgical tool within the subject to find the material. Once the material is found, the operator may initiate a rotation to the carting assembly to debride and cut the cut materials. The process of debriding the material may be reffered to as removing the material. In this example, the material cutting device may be inserted once to complete the laparascopic or hysteroscopic operation or procedure.

The extension of the surgical tool can move towards a treatment site within the subject. The surgical tool may extend or move pass the treatment site, in which an operator can terminate further extension of the surgical tool into the subject. The surgical tool can move towards the treatment site using the surgical tool. While moving towards the treatment site, the operator may push or exert a force to the proximal end of the surgical tool. The surgical tool can be moved further inside the subject and towards the treatment site in response to the force excited to the proximal end.

In some embodiments, the surgical tool or the surgical tool can provide or transmit at least one substance to a treatment site of a subject. The substance can include liquid, gas, or other chemical compounds. The substance may facilitate the process of debriding the material into the cut materials, for example, by exerted a gaseous substance to soften, disperse, or breakdown the material. Accordingly, the provision of the substance can assist the debriding process using the cutting assembly. In another example, the substance may assist in healing the subject, for example, by blocking a damaged portion of the vessel or by providing a medication to the treatment area within the vessel.

At 2420, the distal end (e.g., distal end 1906) of the steerable tubing can be articulates. A first control input can be applied to a first connector (e.g., first connector 1924) coupled to the proximal end of the steerable tubing tot cause articulation of the distal end of the steerable tubing along a longitudinal axis extending through the surgical tool. The first control input can cause the distal end to twist and turn in various angles to maneuver the cutting assembly to the material. For example, the control inputs can bend the proximal end of the steerable tubing by −90, −80, −70, −60, −50, −40, −30, −20, −10, 10, 20, 30, 40, 50, 60, 74, 80, or 90 degrees relative to the longitudinal axis. The first control input can cause the distal end to twist and turn in various angles to maneuver the cutting assembly to the material. The distal end of the surgical tool may be positioned a distance from the material, such as 1 millimeter, 1 inch, or 1 meter from the material.

The first connector tan articulate the distal end on the articulation axis relative to the longitudinal axis. The articulation axis can be relative to the longitudinal axis. For example, the articulation axis can be −90, −80, −70, −60, −50, −40, −30, −20, −10, 10, 20, 30, 40, 50, 64, 70, 80, or 90 degrees relative to the longitudinal axis. The distal end can bend at an angle proportional to the angle bent by the proximal end. For example, the steerable tubing can bend the distal end by 10 degrees responsive to a 10 degree bend of the proximal end by the first connector. In another example, the steerable tubing can bend according to any other configuration. For example, the steerable tubing can bend the distal end by 10 degrees responsive to a 10 degree bend of the proximal end by the first connector, and bend the distal end by 15 degrees responsive to a 20-degree bend of the proximal end by the first connector. In another example, the steerable tubing can bend the distal end by 10-degrees responsive to a 10-degree bend of the proximal end by the first connector, and bend the distal end by 25 degrees responsive to a 20-degree bend of the proximal end by the first connector. The distal end of the steerable tubing may be positioned a distance from the material, such as 1 millimeter, 1 inch, or 1 meter from the material.

In some embodiments, the control input can provide torque at the proximal end of the steerable tubing to rotate the outer sheath. For example, the control inputs can rotate the proximal end by 60-degrees to cause 60-degree rotation of the distal end. In some embodiments, the first connector can input the τ-promixal used to rotate the steerable tubing and thus the outer sheath (or to apply a control torque corresponding to a desired τ-proximal, such as if the first connector includes gars and/or a motorized actuator to drive the rotation, of the steerable tubing). In some embodiments, the first connector receives the torque or the control inputs from a motor. The distal end can rotate with an equivalent torque as the torque provided via the first connector at the proximal end. It should be appreciated that the distal end can be configured to rotate a specific degree, equivalent or matching the degree of rotation of the proximal end. Accordingly, the first connector can provide precision and control of the steerable tubing and thus the outer sheath. For example, the operator may initiate a 30-degrees rotation of the first connector. The rotation, force, and torque can be exerted to the distal end such that the outer sheath also rotates by 30-degrees.

At 2430, the cutting assembly can cut the material front the treatment site. A second control input can be applied to a second connector (e.g., second connector 1932) coupled to the proximal end of the steerable tubing to rotate a flexible torque component (e.g., flexible torque component 1930) disposed within the flexible tubing. In some cases, an operator may exert a manual or mechanism rotation as the second control input applied to the second connector.

The flexible torque component can be coupled to the inner sheath. The inner sheath can be rotated in response to receiving an exerted rotational torte or torque from the second connector. The flexible torque cent can cause the inner sheath to rotate relative to the outer sheath to cut the material. The removal attic material can refer to cutting, debriding, pulling, dissecting, or tearing the material from the treatment site. The cutting assembly can cut the material in response to the rotation by the inner sheath. The torque can be provided by the second connector. The exerted rotation may traverse from the proximal end to the distal end of the steerable tubing. As an example, the second connector may provide 180 degrees rotation to the steerable tubing at a proximal end, and the distal end will rotate by 180 degrees.

At 2440, the material can be retrieved from the treatment site. The substances can include the cut materials, liquid, gas, or other chemical compounds within the body of the subject. The operator can actuate a vacuum source (e.g., vacuum source 1938) coupled to the surgical tool to provide suction through an aspiration channel (e.g., aspiration channel 1934) defined by an inner wan of the flexible tubing to cut the material front the subject via the aspiration channel. The process of retrieving the cut materials may be concurrent to the debriding process by the cutting assembly. For example, the vacuum source can initiate a vacuum to withdraw the cut materials while the cutting assembly debrides the material. The cut materials can be stored in a container or a repository in the vacuum source for external to the surgical tool.

In further example, responsive to the cutting assembly debriding a material into cut materials, the vacuum source may pull in, withdraw, or pump out the cut materials from the subject. The cut materials can be withdrawn via an aspiration channel. The process of withdrawing the cut material can be performed by a vacuum source. The vacuum source can be external to the surgical tool. The vacuum source device can be initiated by a signal or a mechanical trigger. A pump device may be connected to the aspiration channel configured to withdraw of the cut materials from the subject. The pump device may pull a substance front a repository and push the chemical into the subject. The pump device may withdraw the cut materials into a second repository for storage.

The surgical tool may be cut from the subject upon or based on completion of the laparoscopic or hysteroscopic procedures or processes. The completion of the laparoscopic or hysteroscopic procedures can entail the debriding of the material, such as through an entire treatment site, or the collection of the debriding materials. For example, the treatment site can include a length of 3 inches. The surgical tool may initiate a rotation of the cutting assembly and travel through the 3 inches of the treatment site to debride the material. While debriding the materiel, the surgical tool may retrieve or draw in the cut materials into the aspiration channel. For example, once the surgical tool cuts the material through the 3 inch length of the treatment site and retrieve the cut materials, the laparoscopic or hysteroscopic procedures may be completed.

Although the present disclosure discloses various embodiments of a steerable instrument, including but not limited to a tool that may be attached to the tip of the steerable instrument, and a tool that may be fed through the length of the steerable instrument, the scope of the present disclosure is not intended to be limited to such embodiments or to steerable instruments in general. Rather, the scope of the present disclosure extends to any device that may debride and cut materials and/or necrotic materiel from within a body of a subject or a patient using a single tool. As such, the scope of the present disclosure extends to steerable instruments that may be built with some or all of the components of the steerable instruments described herein. Furthermore, it should be understood by those skilled in the art that any or all of the components that constitute the steerable instrument may be built into an existing hysteroscope, laparoscope, endoscope, or into a newly designed material removal tool for use in debriding and removing materials from within the body of the subject.

Having now described some illustrative implementations, it is apparent that the foregoing is illustrative and not limiting, having been presented by way of example. In particular, although many of the examples presented herein involve specific combinations of method sets or system elements, those acts and those elements can be combined in other ways to accomplish the same objectives. Acts, elements and features discussed in connection with one implementation are not intended to be excluded from a similar role in other implementations or implementations.

The phraseology and terminology used herein is for the purpose of description and should not be regarded as limiting. The use of “including,” “comprising,” “having,” “containing,” “involving,” “characterized by,” “characterized in that,” and variations thereof herein, is meant to encompass the items listed thereafter, equivalents thereof, and additional items, as well as alternate implementations consisting of the items listed thereafter exclusively. In one implementation, the systems and methods described herein consist of one, each combination of more than one, or all of the described elements, acts, or components.

Any references to implementations or elements or acts of the systems and methods herein termed to in the singular can also embrace implementations including a plurality of these elements, and any references in plural to any implementation or element or act herein can also embrace implementations including only a single element. References in the singular or plural form are not intended to limit the presently disclosed systems or methods, their components, acts, or elements to single or plural configurations. References to any act or element being based on any information, act or element can include implementations where the act or element is hosed at least in part on any information, act, or element.

Any implementation disclosed herein can be combined with any other implementation or embodiment, and references to “an implementation,” “some implementations,” “one implementation” or the like are not necessarily mutually exclusive and are intended to indicate that a particular feature, structure, or characteristic described in connection with the implementation can included in at least one implementation or embodiment. Such terms as used herein are not necessarily all referring to the same implementation. Any implementation can be combined with any other implementation, inclusively or exclusively, in any manner consistent with the aspects and implementations disclosed herein.

Where technical features in the drawings, detailed description or any claim are followed by reference signs, the reference signs have been included to increase the intelligibility of the drawings, detailed description, and claims. Accordingly, neither the reference signs nor their absence have any limiting effect on the scope of any claim elements.

Systems and methods described herein may be embodied in other specific forms without departing from the characteristics thereof. Further relative parallel, perpendicular, vertical or other positioning or orientation descriptions include variations within +/−10% or +/−10 degrees of pure vertical, parallel or perpendicular positioning. References to “approximately,” “about,” “substantially,” or other terms of degree include variations of +/−10% from the given measurement, unit, or range unless explicitly indicated otherwise. Coupled elements can be electrically, mechanically, or physically coupled to one another directly or with intervening elements. Scope of the systems and methods described herein is thus indicated by the appended claims, rather than the foregoing description, and changes that come within the meaning and range of equivalency of the claims are embraced therein.

The term “coupled” and variations thereof includes the joining of two members directly or indirectly to one another. Such joining may be stationary (e.g., permanent or fixed) or moveable (e.g., removable or releasable). Such joining may be achieved with the two members coupled directly with or to each other, with the two members coupled to each other using a separate intervening member and any additional intermediate members coupled to one another, or with the two members coupled to each other using an intervening member that is integrally formed as a single unitary body with one of the two members. If “coupled” or variations thereof are modified by an additional term (e.g., directly, coupled), the generic definition of “coupled” provided above is modified by the plain language meaning of the additional term (e.g., “directly coupled” means the joining of two members without any separate intervening member), resulting in a narrower definition than the generic definition of “coupled” provided above. Such coupling may be mechanical, electrical, or fluidic.

References to “or,” can be construed as inclusive so that any terms described using “or” can indicate any of a single, more than one, and all of the described terms. A reference to “at least one of ‘A’ and ‘B’” can include only ‘A’, only ‘B’, as well as both ‘A’ and ‘B’. Such references used in conjunction with “comprising” or other open terminology can include additional items.

Modifications of described elements and acts such as variations in sizes, dimensions, structures, shapes and proportions of the various elements, values of parameters, mounting arrangements, use of materials, colors, orientations can occur without materially departing from the teachings and advantages of the subject matter disclosed herein. For example, elements shown as integrally formed can be constructed of multiple parts or elements, the position of elements can be reversed or otherwise varied, and the nature or number of discrete elements or positions can be altered or varied. Other substitutions, modifications, changes and omissions can also be made in the design, operating conditions and arrangement of the disclosed elements and operations without departing from the scope of the present disclosure.

References herein to the positions of elements (e.g., “top,” “bottom,” “above,” “below”) are merely used to describe the orientation of various elements in the FIGURES. It should be noted that the orientation of various elements may differ according to other exemplary embodiments, and that such variations are intended to be encompassed by the present disclosure.

1. A surgical instrument comprising: an outer tubing extending from a proximal end to a distal end along an axis, the distal end comprising an articulation member of the outer tubing;
one or more articulation wires extending along the outer tubing and coupled to the articulation member;
a cutting assembly coupled to the distal end of the outer tubing, the cutting assembly including an outer component and an inner component disposed within the outer component coupled to the articulation member, the outer component defining a cutting window configured to cut material;
a flexible torque component having a portion disposed within the outer tubing, the flexible torque component coupled to the inner component and configured to rotate the inner component relative to the outer component to cut the material; and
a handle comprising a first actuator to rotate the outer tubing to adjust a position of the cutting window about the axis, the handle including a second actuator coupled to the one or more articulation wires to articulate the articulation member coupled to the cutting assembly away from the axis.

2. The surgical instrument of claim 1, wherein the axis is a first axis, and wherein the inner component rotates about a second axis relative to the first axis, the second axis formed by articulating the articulation member.

3. The surgical instrument of claim 1, further comprising a tensioning rod disposed along the outer tubing, the tensioning rod configured to maintain a tension of the one or more articulation wires to control rotation and articulation of the articulation member coupled to the cutting assembly.

4. The surgical instrument of claim 1, wherein a proximal end of the flexible torque component is coupled to a motor configured to transmit torque to the proximal end of the flexible torque component, the flexible torque component configured to transmit torque from the proximal end to the distal end to cause the inner component to rotate relative to outer component to cut the material.

5. The surgical instrument of claim 4, further comprising an aspiration channel having an aspiration port configured to engage with a vacuum source, the aspiration channel partially defined by the flexible torque component and extending from the cutting window defined by the cutting assembly to the aspiration port.

6. The surgical instrument of claim 1, wherein the first actuator is configured to articulate the distal end at a first angle of rotation proportional to a second angle of rotation of the first actuator.

7. The surgical instrument of claim 1, wherein the second actuator is a plurality of actuators, each of the plurality of actuators coupled to a corresponding wire of the one or more articulation wires.

8. The surgical instrument of claim 1, further comprising a sheath enclosing the one or more articulation wires.

9. The surgical instrument of claim 1, wherein the handle further comprises a locking assembly configured to restrict movement of at least one of the one or more articulation wires to set the cutting assembly to a predetermined articulation.

10. The surgical instrument of claim 1, wherein the one or more articulation wires are a first set of one or more articulation wires, and wherein the surgical instrument further comprises:
a second set of one or more articulation wires oriented at a first angle relative to the first set of one or more articulation wires;
a third set of one or more articulation wires oriented at a second angle relative to the first set of one or more articulation wires; and
wherein the second actuator is coupled to the first, second, and third set of one or more articulation wires.

11. A surgical instrument comprising:
an outer tubing extending from a proximal end to a distal end along an axis, the distal end comprising a plurality of segments;
one or more articulation wires extending along the outer tubing and coupled to the plurality of segments; a cutting assembly coupled to the distal end of the outer tubing, the cutting assembly configured to cut material from a subject; and
a handle comprising a first actuator to rotate a first component of the cutting assembly about the axis and a second actuator coupled to the one or more articulation wires to selectively articulate at least one of the plurality of segments coupled to the cutting assembly away from the axis.

12. The surgical instrument of claim 11, wherein the first component is an outer component of the cutting assembly and the cutting assembly further comprises an inner component disposed within the outer component, the outer component defining a cutting window.

13. The surgical instrument of claim 11, further comprising a tensioning rod disposed along the outer tubing, the tensioning rod configured to maintain a tension of the one or more articulation wires to control rotation and articulation of the articulation member coupled to the cutting assembly.

14. The surgical instrument of claim 12, further comprising a flexible torque component having a portion disposed within the outer tubing, the flexible torque component coupled to the inner component and configured to rotate the inner component relative to the outer component to cut the material.

15. The surgical instrument of claim 14, further comprising an aspiration channel having an aspiration port configured to engage with a vacuum source, the aspiration channel partially defined by the flexible torque component and extending from the cutting window defined by the cutting assembly to the aspiration port.

16. The surgical instrument of claim 11, wherein the first actuator is configured to articulate the distal end at a first angle of rotation proportional to a second angle of rotation of the first actuator.

17. The surgical instrument of claim 11, wherein the second actuator is a plurality of actuators, each of the plurality of actuators coupled to a corresponding wire of the one or more articulation wires.

18. The surgical instrument of claim 11, further comprising a sheath enclosing the one or more articulation wires.

19. The surgical instrument of claim 11, wherein the handle further comprises a locking assembly configured to restrict movement of the one or more articulation wires to set the cutting assembly to a predetermined articulation.

20. The surgical instrument of claim 11, wherein the outer tubing includes a plurality of securing elements extending from the plurality of segments, the plurality of securing elements configured to secure the one or more articulation wires to the outer tubing.

21. The surgical instrument of claim 20, further comprising a sheath enclosing the one or more articulation wires and the plurality of securing elements.

22. The surgical instrument of claim 11, wherein the one or more articulation wires are a first set of one or more articulation wires, and wherein the surgical instrument further comprises:
a second set of one or more articulation wires oriented at a first angle relative to the first set of one or more articulation wires;
a third set of one or more articulation wires oriented at a second angle relative to the first set of one or more articulation wires; and
wherein the second actuator is coupled to the first, second, and third set of one or more articulation wires.

23.-62. (canceled)
